(19) United States
(12) Patent Application Publication
(10) Pub. No.: US 2008/0311092 A1
(43) Pub. Date: Dec. 18, 2008

(54) Murine Stem Cells and Applications Thereof

(76) Inventors: Isidro Sanchez-Garcia, Salamanca (ES); Maria Perez-Caro, Salamanca (ES); Felipe Voces-Sanchez, Salamanca (ES)

Correspondence Address: MORRISON & FOERSTER LLP, 425 MARKET STREET, SAN FRANCISCO, CA, 94105-2482, US

(21) Appl. No.: 11/915,318

(22) PCT Filed: May 24, 2006
(86) PCT No.: PCT/IB2006/001969

§ 371 Date: Jul. 14, 2008

(30) Foreign Application Priority Data
May 24, 2005 (EP) 05076219.4

(51) Int. Cl. A61K 35/12; A01K 67/027; C12N 5/02; C12N 5/10; C12Q 1/68; A61P 35/00; C12N 15/11; G01N 33/574; C12Q 1/02

(52) U.S. Cl. 424/93.21; 800/10; 435/378; 435/325; 800/3; 435/29; 800/21; 435/6

(57) ABSTRACT

The invention relates to animal solid tumour models which comprise a transgenic non-human mammal containing in its genome a DNA construct that comprises a gene created and/or activated by a genetic anomaly associated with human cancer operatively bound to a promoter that directs the expression of the gene in Sca1+ cells. The invention also relates to stem cells capable of specifically expressing in stem cells human genetic anomalies associated with human pathologies. Applications of these models and stem cells, such as diagnostic, therapeutic and prophylactic applications for human diseases, and products and methods are provided.

### FIELD OF THE INVENTION

The invention relates to murine stem cells capable of specifically expressing in stem cells human genetic anomalies associated with human pathologies. Applications of said stem cells, such as human disease diagnostic, therapeutic and prophylactic applications, products and methods are provided.

### BACKGROUND OF THE INVENTION

Stem cells (SC) are defined as cells that have the ability to perpetuate themselves through self-renewal and to generate mature cells of a particular tissue through differentiation. Examples of SCs include haematopoietic stem cells, neural stem cells, cancer stem cells, etc.

Nowadays, it is thought that cancers consist of heterogeneous populations of cancer cells that differ markedly in their ability to proliferate and form new tumours. While the majority of the cancer cells have a limited ability to divide, a population of cancer stem cells (CSC) which has the exclusive ability to extensively proliferate and form new tumours can be identified based on marker expression.

Growing evidence suggests that pathways regulating the self-renewal of normal stem cells are deregulated in CSCs resulting in the continuous expansion of self-renewing cancer cells and tumour formation. This suggests that agents targeting the defective self-renewal pathways in cancer cells might lead to improved outcomes in the treatment of these diseases.

This strategy has implications for the biology of tumour formation as well as the diagnosis and treatment of cancer. To treat cancer effectively, the CSCs must be eliminated. Otherwise, the tumour will rapidly reform if the therapy eliminates non-tumorigenic cancer cells but spares a significant population of the CSCs. Classically, treatments for cancer have relied on the ability to shrink tumours. Since in many cases the CSCs represent a minority cell population of the tumour, agents selectively killing the CSCs are likely overlooked in current screening methods, which rely on rapid reduction of tumour size.

If an agent spares a significant number of the CSCs, then the remaining cells could rapidly reform the tumour. In addition to its effect on our understanding of the efficacy of our current therapies, the stem cell model for cancer is likely to affect the identification of future therapeutic targets. By directing expression analyses to enriched population of tumorigenic cancer cells, the identification of novel diagnostic markers and novel therapeutic targets should be more effective. In addition, it is becoming apparent that treatments that directly target the pathways involved in maintenance of CSCs would have a significantly greater chance of success.

If it is assumed that the genetic alteration responsible for the cancer development takes place in the CSCs, mouse models based on this issue can be designed. New strategies must focus on determining the process that is responsible for the maintenance of the CSC phenotype. This will be the aim for the new identification of targets to design drugs against them. Therefore, mouse models generated in this way will be the basic tool to design drugs in this context, directed against the maintenance of the CSCs.

Many new anticancer agents target unconventional aspects of cancer development and interact with other drugs in an unpredictable manner. They are predicted to show clinical benefit in only small subpopulations of patients

In addition, in order to improve the efficacy of current therapies, the stem cell model for cancer will also help for the identification of potential therapeutic targets. By directing expression analyses to enriched population of tumorigenic cancer cells, the identification of novel diagnostic markers and novel therapeutic targets will be more effective. Recent evidence also indicates that treatments directly targeting the pathways involved in maintenance of CSCs will have significantly greater chances of success.

Based on the understanding that the genetic alteration responsible for cancer development takes place in the CSCs, it is possible to design mouse models that accurately reproduce this genetic alteration. First, we must determine what is responsible for the maintenance of the CSC phenotype since this will be the basis for the identification of targets and the potential drugs to be used against them. Therefore, animal models such as mouse models generated following this strategy will represent the basic tool in designing drugs to inhibit CSC maintenance.

### DESCRIPTION OF THE INVENTION

In one aspect, the invention relates to an animal cancer model, the model comprising a transgenic non-human mammal containing in its genome a DNA construct that comprises a gene created and/or activated by a genetic anomaly associated with a human pathology, operatively bound to a promoter that directs the expression of the gene in Sca1⁺ cells. The gene created and/or activated by a genetic anomaly is herein referred to as an activatable gene. As a result of this construct, the transgenic non-human mammal contains a stem cell (SC) that comprises and expresses the activatable gene.

One aspect of the invention relates to a stem cell (SC), optionally isolated from such a transgenic non-human mammal, comprising a gene created and/or activated by a genetic anomaly associated with a human pathology. In a particular embodiment, said SC is a cancer stem cell (CSC). The SC or CSC may be, among others, a murine SC or CSC.

Preferably, according to the above aspects of the invention, the human pathology is a cancer, preferably a solid tumour. A few examples of solid tumours include mesenchymal cancers, e.g. sarcomas; epithelial cancers e.g. carcinomas; lymphomas and so on.

The inventors have discovered that in animal models, CSC generated cancer faithfully replicates the equivalent human cancer phenotypes. Current animal models, principally mouse models, fail to reproduce genotype-phenotype correlations of human cancer. In these models, human cancer genetic defects that are introduced into mice lead to a mouse model in which the cancer genetic defect is present either in all cells or in specific differentiated cells. Such mouse models are generated without considering the cancer stem cell model and thus without taking into account the nature of the correct human cancer target cells. Accordingly, these models poorly reproduce human cancer genotype-phenotype correlations and respond to drugs differently to the response patterns found in human cancer patients. Indeed, it is the inventors' contention that selection and/or testing of targets and drug candidates in such conventional models is unlikely to be predictive of the human response.

For example, when H-Ras^{V12G} is expressed in the mouse, it leads to a mouse phenotype reflective of a melanoma. In contrast, the human phenotype is bladder, kidney and thyroid carcinoma. Gene deletion in the mouse, for example, using farnesyl transferase inhibitors cures the mouse model of cancer. In contrast, the same product gives no relief in humans.

A second example can be provided by BCR-ABL^{p210} which leads to B cell acute lymphoblastic leukaemia (B-ALL) in mouse models. In humans, the phenotype is altogether different (chronic myeloid leukaemia, CML). In the mouse B-ALL model, Gleevec™ cures the cancer, whilst in humans this product only gives symptomatic relief. It thus appears that mere untargeted introduction of a gene defect linked to a cancer into a mouse model does not lead to the establishment of a disease state that mirrors the human condition in any way, probably because the gene is not correctly targeted to the cells in the mouse that are responsible for generating the disease.

The dire need for new animal models of cancer is supported by expert comment in the field—it is now a well-documented fact that current animal models are inadequate and that new mouse models are a key to any fundamental improvement in cancer therapy. For example, Robert Weinberg has stated (March 2004): “A fundamental problem which remains to be solved in the whole cancer research effort, is that the preclinical models of human cancer are essential”. In Fortune magazine, (Mar. 22, 2004) he stated “To win the war we need models that better mimic human response and biomarkers that are predictive of cancer development”.

The question asked by the present inventors inquired as to why so many mouse models of cancer are inadequate. The answer, they believe, lies in the answer that cancer is not a proliferation disease and that cancer stem cells provide the true cellular target for human cancer. CSCs are considered to possess properties and pathways that are unique and different from the cells that form the bulk of a tumour. It has been established by the inventors that targeting a genetic anomaly associated with a human pathology into a somatic stem cell establishes an animal model that precisely mirrors the human pathological condition. In addition to leukaemias, these animal models generate solid tumours. This is highly surprising, and most advantageous.

Desired properties of a cancer stem cell animal model must recapitulate human cancer in that animal. Such desired properties include establishment of similar histological features to human cancer; they should progress through the same disease stages; they should cause the same systemic effects on the host; they should cause the same genetic pathways in tumour initiation/progression; and they should respond in the same way as the human to current therapeutic approaches.

Imbued with this knowledge, the inventors have generated multiple animal models in which human oncogenes are expressed in stem cells. These models have been found to mimic human cancer, both in their histopathology and their response to therapeutic agents. These models can be used in an integrated approach to discover novel therapeutic approaches; discover new methods to assess therapeutic efficacy; discover and optimize new diagnostic tools; as a source of CSC for screening and to test novel and existing drugs/drug candidates. All of these approaches and models form embodiments of the present invention.

To facilitate the understanding of the instant description, the meaning of some terms and expressions in the context of the invention will be explained below.

The term “stem cell” (SC) refers to a cell that has the ability to perpetuate itself through self-renewal and to generate mature cells of a particular tissue through differentiation. They are self-renewing tissue cells that are multipotent and tightly controlled. Examples of SCs include haematopoietic stem cells, neural stem cells, sarcoma stem cells, breast stem cells, lung stem cells, brain stem cells, prostate stem cells, pancreatic stem cells, and so on.

It is the inventors' contention that cancer stem cells (CSCs) are responsible for both tumour recurrence and metastasis and provide a novel cellular target that will provide novel molecular targets. Cancer stem cells express specific markers that are characteristic of cells in the stem cell compartment and these will be known to those of skill in the art. For example, stem cells are Sca1⁺. Stem cells are also Lin⁻.

The term “cancer stem cell” (CSC) refers to a cell that has the ability to extensively proliferate, form new tumours and maintain cancer development, i.e., cells with indefinite proliferative potential that drive the formation and growth of tumours; said term includes both gene alteration in SCs and gene alteration in a cell which becomes a CSC. In a preferred embodiment, said CSCs are Sca1⁺.

The term “subject” refers to vertebrates, including members of the mammal species, and includes, but is not limited to, domestic animals, rodents (particularly murine animals), primates and humans; the subject is preferably a human being, male or female, of any age or race.

The term “murine” includes mice, rats, guinea pigs, hamsters and the like; in a preferred embodiment the murine animal is a mouse.

The expression “gene that is created and/or activated by a genetic anomaly associated with a human pathology”, hereinafter, referred to as the “activatable gene”, refers to a gene, altered gene (including mutations, deletions, insertions, duplications, etc.) or gene fusion that, when incorporated into the genome of a mammal, reproduces the human pathology with which said gene, altered or gene fusion is associated. Preferably, activatable genes according to the invention are oncogenes. Many oncogenes are known, and are linked to particular types of cancer. For example, BCR-ABLp210 is related to chronic myeloid leukaemia. BCR-ABLP^{p190}, TEL-AML1, E2A-HLF and E2A-Pbx1 are related to B-cell acute lymphoblastic leukaemia (B-ALL). Other examples are given below and further examples still will be known to those of skill in the art. For example, comprehensive lists of oncogenes are provided at: http://www.infobiogen.fr/services/chromcancer/Genes/Geneliste.html; see also Cooper G. Oncogenes. Jones and Bartlett Publishers, 1995; Vogelstein B, Kinzler K W. The Genetic Basis of Human Cancer. McGraw-Hill: 1998; http://cancerquest.org/index.cfm?page=780.

The term “human pathology” includes human pathology of stem cell origin as well as human pathology of non stem cell origin. In a particular embodiment, the human pathology is a human pathology of stem cell origin. In another particular embodiment, said activatable gene is a gene that is created and/or activated by a genetic anomaly associated with a human pathology of stem cell origin such as a human tumoural pathology or a human non-tumoural pathology.

In a further particular embodiment said human pathology is a solid tumour. The human pathology may be selected from epithelial or mesenchymal cancer. Particular examples of such cancer include myeloproliferative disorders, human lymphomas (e.g. Burkitt-like lymphoma, Diffuse Large B-Cell Lymphoma (DLBCL) and marginal zone lymphoma), leukaemias, including chronic myeloid leukaemia, B-cell acute lymphoblastic leukaemia (B-ALL), T-cell acute lymphoblastic leukaemia (T-ALL), acute myeloid leukaemia (AML), chronic myeloid leukaemia (CML), lymphoproliferative syndromes, including multiple myeloma, non-Hodgkin lymphoma, leukopenia, thrombocytopenia, angiogenesis disorder; any sarcoma, including Kaposis' sarcoma, liposarcoma, Ewing sarcoma, any mesenchymal cancer; any carcinomas, including lung carcinoma (e.g. small cell carcinoma of the lung (SCLC), non-small cell carcinoma of the lung (NSCLC), and lymphoma, breast carcinoma, skin carcinoma and kidney carcinoma. Other examples include neoplasm; melanoma; and solid tumours, for example, of the lung, colorectal, breast, uterus, prostate, pancreas, head and neck, brain.

In another particular embodiment, said activatable gene is a gene that is created and/or activated by a genetic anomaly associated with a human pathology such as haematopoietic or embryonic stem cell migration. In another particular embodiment, said activatable gene is a gene that is created and/or activated by a genetic anomaly associated with a human pathology such as neurological disorders, rare diseases, metabolic diseases, immunological diseases, cardiovascular diseases, etc.

Accordingly, the invention embraces models of human cancer in which a human pathology is generated in an animal model, particularly in a mouse model. Separate embodiments of the invention relating to this aspect provide models of epithelial or mesenchymal cancer. Examples of such models include models of human lymphomas, leukaemias, sarcomas and carcinomas, in particular, chronic myeloid leukaemia, B-cell acute lymphoblastic leukaemia (B-ALL), T-cell acute lymphoblastic leukaemia (T-ALL), acute myeloid leukaemia (AML), chronic myeloid leukaemia (CML), lymphoproliferative syndromes, multiple myeloma, sarcomas, for example, liposarcoma, Ewing sarcoma, carcinomas, for example, lung carcinoma, breast carcinoma, skin carcinoma and kidney carcinoma, that are described herein.

Animal models may be generated according to this aspect of the invention by generating in the animal a stem cell (SC) comprising a gene created and/or activated by a genetic anomaly associated with a human pathology. The inventors have established that these models all share features that are necessary in any faithful reproduction of the cancer state, including establishment of similar histological features to human cancer; progression through the same disease stages; they cause the same systemic effects on the host; they activate/depress the same genetic pathways in tumour initiation/progression; and they respond in the same way as humans to various therapeutic approaches.

In order to generate a model of cancer according to the present invention, the gene created and/or activated by the genetic anomaly associated with the human pathology (the activatable gene) is preferably operatively bound to a promoter that directs the expression of said genetic anomaly in Sca1⁺ cells. Such a promoter is a sequence of nucleic acid implicated and necessary in the initiation of transcription, which directs the expression of the activatable gene in said cells, and which includes the binding site of RNA polymerase. Within the context of the present invention, the term “promoter” may include other sites to which the transcription regulating proteins can bind.

In preferred embodiments of the invention, this promoter may be any eukaryotic promoter that directs the expression of the genetic anomaly in Sca1⁺ cells i.e. any promoter that is only active in the stem cell compartment and which is inactive once cells differentiate beyond the stem cell state. Promoters that may be active in the stem cell compartment, but are also active in differentiated cells, are unsuitable for use in the present invention. The specificity of the promoter activity only to stem cells is an important aspect of the invention. Preferably, the promoter is one which is active in cells that express Sca1, optionally, also in cells that express human epithelial antigen (HEA) or carcinoembryonic antigen (CEA) in humans, CD133; α2β1 integrin and so on. The promoter is preferably inactive in cells that are Lin+.

For example, the promoter may be any eukaryotic Sca1 promoter, particularly a Sca1 promoter derived from a vertebrate, an animal, a mammal, a rodent or a mouse. Another example of a suitable promoter includes the musashi-1 and the musashi-2 promoters and functional equivalents thereof (Siddall N A et al. Proc. Natl Acad. Sci. USA 103: 8402-8407, 2006). Details of the mouse musashi-1 gene may be found at GeneID: 17690 Primary source: MGI:107376 in Entrez Gene (http://www.nobi.nlm.nih.gov/).

In a particular embodiment that the inventors have used to demonstrate the present invention, the promoter that directs the expression of the activatable genes in Sca1⁺ cells may be the pLy-6E1 promoter of mouse or a functional fragment thereof or a functional equivalent thereof. In other words, it is able to direct the tissue specific expression of the different transgenes in the animal model, such as in this instance in mice. The pLy-6E1 promoter is well characterised and contains all the elements necessary for the selective expression in Sca1⁺ cells [Miles C., Sanchez M-J, Sinclair A, and Dzierzak, E. (1997) “Expression of the Ly-6E.1 (Sca-1) transgene in adult haematopoietic stem cells and the developing mouse embryo”. Development 124:537-547]. Thus, in a particular embodiment, the promoter that directs the expression of said activatable gene in Sca1⁺ cells is the mouse promoter pLy-6E.1 or a functional fragment or equivalent thereof. Functional equivalents of this promoter will be well known to those of skill in the art and include promoters of the pLy6A gene, Tmtsp gene, c-kit gene, mouse CD34 gene, Thy1 gene, etc.

The expression “operatively bound” relates to the orientation of the promoter with respect to sequence of activatable gene. The promoter is placed such that it is able to control or regulate the expression of the activatable gene.

Illustrative, non limitative examples of said activatable gene include the genes identified in the art as BCR-ABL^{p210}, BCR-ABL^{p190}, Slug, Snail, HOX11, RHOM2/LMO-2, TAL1, Maf-B, FGFR, c-maf, MMSET, BCL6, BCL10, MALT1, cyclin D1, cyclin D3, SCL, LMO1, LMO2, TEL-AML1, E2A-HLF, E2A-Pbx1, TEL-ABL, AML1-ETO, FUS-DDIT3, EWS-WT1, EWS FLI1, EWSR1-DDIT3, FUS-ATF1, FUS-BBF2H7, K-RASv12, Notch1, etc. Information concerning BCR-ABL^{p210}, BCR-ABL^{p190}, Slug, Snail, HOX11, RHOM2/LMO-2 and TAL1 genes can be found in WO 03/046181. Sequences for all these genes and gene fusions can be simply accessed from existing databases by the person of skill in the art, for example, at http://www.ncbi.nlm.nih.gov/entrez/query.fcgi?db=gene. As the skilled person will appreciate, the exact sequence of any one of these genes that is used may vary. Preferably, a mammalian activatable gene is used, such as a mouse sequence, more preferably a human sequence. However, the invention includes the use of variant activatable genes so as to include, for example, polymorphic variants, mutants and other gene types not considered wild type. For example, such genes could be used to investigate differences in the oncogenic potency of such genes that might reflect differences in patterns of disease and response to treatment across patient groups and so on. Accordingly, reference to each of the genes referred to above includes within its scope, references to variants of such genes, for example, that share significant homology or sequence identity of 80%, 85%, 90%, 95%, 98%, 99% or more with the sequences represented in the accession numbers presented above. The gene may be cDNA, or may comprise genomic sequence. cDNA is preferred.

It is shown herein that oncogenic activation in solid tissue Sca1⁺ cells, leads to solid tissue carcinomas. This work has been exemplified in mice, using a conditional system in which AdenoCre is used conditionally to activate an oncogene (in this case, human KRASv12, one preferred example). In this system, non-small cell lung cancer (NSCLC) lung carcinomas were observed around 3-5 months after AdenoCre infection i.e. after oncogenic activation.

It is thus demonstrated herein that targeting of gene defects, linked to human hematopoietic cancers, to Sca1⁺ cells in solid tissue leads to solid tumor development.

For example, the inventors' models of CML (see below) have been shown to generate 79% of mice with CML disease only. The remainder are composed of CML and lung adenocarcinoma (lung ADC) (12%), liver adenocarcinoma (liver ADC) (3%), fibrous histiocytoma (2%), osteosarcoma (2%) and Sertoli cell tumours (2%).

Accordingly, this aspect of the invention relates to a transgenic non-human mammal that contains in its genome a DNA construct that comprises an activatable gene (i.e., a gene created and/or activated by a genetic anomaly associated with a human pathology) operatively bound to a promoter that directs the expression of the genetic anomaly in Sca1⁺ cells, which generates or has the potential to generate a solid tumour. Examples of solid tumours include myeloproliferative disorders, human lymphomas (e.g. Burkitt-like lymphoma, Diffuse Large B-Cell Lymphoma (DLBCL) and marginal zone lymphoma), lymphoproliferative syndromes, including multiple myeloma, non-Hodgkin lymphoma, leukopenia, thrombocytopenia, angiogenesis disorder; any sarcoma, including Kaposis' sarcoma, liposarcoma, Ewing sarcoma, any mesenchymal cancer; any carcinomas, including lung carcinoma (e.g. small cell carcinoma of the lung (SCLC), non-small cell carcinoma of the lung (NSCLC)), breast carcinoma, skin carcinoma, colorectal, uterus, prostate, pancreas, head and neck, brain and kidney carcinoma, neoplasm; melanoma; etc.

The inventors' models of T-ALL using Sca1⁺Rhom2 mice generate 74% of T cell or HSC disease only, with the remainder being composed of the hematopoietic disease and lung adenocarcinoma (16%), liver carcinoma (5%) and others (5%). Sca1⁺Hox11 mice have a similar pattern.

The inventors' models of B cell lymphoma using Sca1⁺BCL6 mice generate 86% of lymphoma disease only, with the remainder being composed of lymphoma and lung adenocarcinoma (9%), and liver carcinoma (3%).

BCR-ABL^{p210} is related to chronic myeloid leukaemia. The inventors have established that an animal model of CML, leading in certain instances also to solid tumor development, can be generated by incorporating BCR-ABL^{p210} into the stem cell compartment of the animal. The construct encoding BCR-ABL^{p210} should be arranged such that BCR-ABL^{p210} transcripts are generated in the stem cell compartment, specifically, in Sca1⁺Lin⁻ cells.

Such a model has been found to recapitulate the various features of the human CML pathology. For example, a mouse model generated by the inventors and described herein shows a chronic (myeloproliferative) phase and blast crisis. The size of the haematopoietic stem cells is normal. Furthermore, Gleevec™ is ineffective to treat CML, and provides only symptomatic relief, as is the case in the human condition. This was unexpected but provides good evidence for the credibility of the model as a realistic model that reproduces relevant features of the human CML pathology.

The CML model according to the invention may be established by introducing BCR-ABL^{p210} into the stem cell compartment of the animal. The BCR-ABL^{p210} sequence may be operatively bound to a promoter that directs the expression of the sequence in Sca1⁺ cells. An example of such a promoter suitable for the generation of a mouse model is the mouse promoter pLy-6E.1 or a functional fragment or equivalent thereof. Other examples will be clear to those of skill in the art on reading the present specification.

In such a model, it is found that the percentage of BCR-ABL^{p210} transcripts in Sca1⁺Lin⁻ cells is high, and low in Sca1⁻Lin⁺ cells, after the stem cells have differentiated beyond the stem cell state. In support of the CSC theory put forward by the present inventors, it is also found that the pattern of BCR-ABL^{p210} transcripts is no different before and after onset of CML in both Sca1⁺Lin⁻ cells and Sca1⁻Lin⁺ cells. This supports the contention that BCR-ABL^{p210} itself has no direct role in the cancer state, but causes further downstream genetic/epigenetic modification (by imposing a cancer cell imprinting) that is actually the real cause of cancer disease.

To the inventor's knowledge, this stem cell driven CML development is the first mouse model to reproduce human CML and blast crisis. In a similar manner to the pattern of disease seen in the human state, these models progress spontaneously from CML to blast crisis. Furthermore, megakaryocytes in the spleen and liver of these animals define myeloid metaplasia. FACS analysis also demonstrates accumulation of mature myeloid cells in peripheral blood. Furthermore, the mouse model mirrors the DNA methylation changes that are associated with malignant transformation in man.

BCR-ABL^{p190}, TEL-AML1, E2A-HLF and E2A-Pbx1 are related to B-cell acute lymphoblastic leukaemia (B-ALL). The inventors have established that animal models of this disease, leading in certain instances also to solid tumor development, can be generated by incorporating any one of BCR-ABL^{p190}, TEL-AML1, E2A-HLF or E2A-Pbx1 into the stem cell compartment of the animal. It has been found that models based on the creation of transgenic mice containing any one of these genes faithfully recapitulate the various features of the human B-ALL pathology. The oncogenic sequence may be operatively bound to a promoter that directs the expression of the sequence in Sca1⁺ cells, such as the mouse promoter pLy-6E.1 or a functional fragment or equivalent thereof, as discussed above.

A prototypic example of a B-ALL model is demonstrated herein in the mouse, in which the characteristic features of the human pathology are mirrored in the mouse system. In particular, extensive human B cell blast infiltration into peripheral blood, liver, spleen and lung is demonstrated.

SCL, HOX11, LMO1, and LMO2 are related to T-cell acute lymphoblastic leukaemia (T-ALL). The inventors have established that animal models of T-ALL, leading in certain instances also to solid tumor development, can be generated by incorporating any one of SCL, HOX11, LMO1, or LMO2 into the stem cell compartment of the animal. It has been found that models based on the creation of transgenic mice containing any one of these genes faithfully recapitulate the various features of the human T-ALL pathology. For example, the LMO2 sequence may be operatively bound to a promoter that directs the expression of the sequence in Sca1⁺ cells, such as the mouse promoter pLy-6E.1 or a functional fragment or equivalent thereof, as discussed above.

A prototypic example of a T-ALL model is demonstrated herein in the mouse, in which the characteristic features of the human pathology are mirrored in the mouse system. In particular, T cell leukaemia development and blast dissemination into tissues and peripheral blood is demonstrated, identical to the human pathology.

Maf-B, FGFR, c-maf, and MMSET genes are related to multiple myeloma. The inventors have established that an animal model of multiple myeloma, leading in certain instances also to solid tumor development, can be generated by incorporating any one of Maf-B, FGFR, c-maf, or MMSET into the stem cell compartment of the animal. It has been found that all these models faithfully recapitulate the various features of the human B-ALL pathology. For example, any one of the Maf-B, FGFR, c-maf, or MMSET sequences may be operatively bound to a promoter that directs the expression of the sequence in Sca1⁺ cells, such as the mouse promoter pLy-6E.1 or a functional fragment or equivalent thereof, as discussed above.

A prototypic example of a multiple myeloma model is demonstrated herein in the mouse, in which the characteristic features of the human pathology are mirrored in the mouse system. In particular, plasma cell infiltration is demonstrated into spleen and kidney and pathological indications of kidney failure are seen. Plasma cells are also demonstrated in bone marrow and peripheral blood, in a pathology very similar to that seen in the human.

BCL6, BCL10, MALT1, cyclin D1 and cyclin D3 are related to lymphoproliferative disorders such as Burkitt-like lymphoma, Diffuse Large B-Cell Lymphoma (DLBCL) or marginal zone lymphoma. The inventors have established that animal models of these diseases can be generated, leading in certain instances also to solid tumor development, by incorporating BCL6, BCL10, MALT1, cyclin D1 or cyclin D3 into the stem cell compartment of the animal. It has been found that models based on the creation of transgenic mice containing any one of these genes faithfully recapitulate the various features of human lymphomas. For example, any one of these sequences may be operatively bound to a promoter that directs the expression of the sequence in Sca1⁺ cells, such as the mouse promoter pLy-6E.1 or a functional fragment or equivalent thereof, as discussed above.

A prototypic example of such a model has been generated using the BCL6 gene, incorporated into the stem cell compartment of the animal under the operative control of a promoter that directs the expression of the sequence in Sca1⁺ cells. It is found that the percentage of BCL6 transcripts in Sca1⁺Lin⁻ cells is high, and low in Sca1⁻Lin⁺ cells, after the stem cells have differentiated beyond the stem cell state. In support of the CSC theory put forward by the present inventors, it is also found that the pattern of BCL6 transcripts is no different before and after onset of lymphoma in both Sca1⁺Lin⁻ cells and Sca1⁻Lin⁺ cells. This supports the contention that BCL6 itself has no direct role in the cancer state, but causes further downstream genetic/epigenetic modification (by imposing a cancer cell imprinting) that is actually the real cause of cancer disease.

The disease model mirrors the pathology of the disease that is found in the human. For example, the diffuse large B cell lymphoma in spleen in the model exactly mimics that seen in the human. Pax5 and CD21+ve B cells infiltrate into the lung and kidney. Indeed, these B cells in Sca1⁺BCL6 mice do not express BCL6, adding further weight to the contention that the targeting of BCL6 is a redundant exercise in the treatment of cancer. Furthermore, it has been found that crossing a Sca1⁺BCL6 mouse with a Bcl6-deficient mouse cannot rescue the phenotype.

More interestingly still, the B cell lymphomas generated in the mice are molecularly similar to human B cell lymphomas. For example, classic molecular prognostic markers that are used to classify human DLBCL are present in the DLBCL model generated and described herein. Such markers include Mdm2, Bcl-2, p27-Ccnd3, Ezh2-Bmi1, c-Myc, Foxp1 and Pde4b. Animal models characterised by possessing B cell lymphomas with this pattern of expression form a further aspect of the present invention.

Furthermore, B cell lymphomas in the models according to the present invention have a similar molecular signature to human ABC-DLBCL. Neither ABC-DLBCL in humans (Alizadeh, A A. et al. Nature, 2000; 403:503) nor in these mouse models express BCL6. Additionally, Sca1⁺Bcl6 mice show a plasma cell differentiation blockade similar to those observed in human ABC-DLBCL.

TEL-ABL, AML1-ETO and FUS-ERG are related to acute myeloid leukaemia. The inventors have established that animal models of these diseases can be generated by incorporating TEL-ABL, AML1-ETO or FUS-ERG into the stem cell compartment of the animal. It has been found that models based on the creation of transgenic mice containing any one of these genes faithfully recapitulate the various features of the human myeloid leukaemia pathology. For example, any one of these sequences may be operatively bound to a promoter that directs the expression of the sequence in Sca1⁺ cells, such as the mouse promoter pLy-6E.1 or a functional fragment or equivalent thereof, as discussed above.

EWS-WT1 and EWS FLI1 are related to Ewing sarcoma. The inventors have established that animal models of Ewing sarcoma can be generated by incorporating EWS-WT1 or EWS FLI1 into the stem cell compartment of the animal. It has been found that models based on the creation of transgenic mice containing any one of these genes faithfully recapitulate the various features of the human Ewing sarcoma pathology. For example, any one of these sequences may be operatively bound to a promoter that directs the expression of the sequence in Sca1⁺ cells, such as the mouse promoter pLy-6E.1 or a functional fragment or equivalent thereof, as discussed above.

FUS-DDIT3 and EWSR1-DDIT3 are related to myxoid liposarcoma. The inventors have established that animal models of myxoid liposarcoma can be generated by incorporating FUS-DDIT3 or EWSR1-DDIT3 into the stem cell compartment of the animal. It has been found that models based on the creation of transgenic mice containing any one of these genes faithfully recapitulate the various features of the human myxoid liposarcoma pathology. For example, any one of these sequences may be operatively bound to a promoter that directs the expression of the sequence in Sca1⁺ cells, such as the mouse promoter pLy-6E.1 or a functional fragment or equivalent thereof, as discussed above.

FUS-ATF1 is related to angiomatoid fibrous histiocytoma. The inventors have established that animal models of angiomatoid fibrous histiocytoma can be generated by incorporating FUS-ATF1 into the stem cell compartment of the animal. It has been found that models based on the creation of transgenic mice containing this gene faithfully recapitulate the various features of the human angiomatoid fibrous histiocytoma pathology. For example, this sequence may be operatively bound to a promoter that directs the expression of the sequence in Sca1⁺ cells, such as the mouse promoter pLy-6E.1 or a functional fragment or equivalent thereof, as discussed above.

FUS-BBF2H7 is related to low grade fibromyxoid sarcoma (LGFMS). The inventors have established that animal models of LGFMS can be generated by incorporating FUS-BBF2H7 into the stem cell compartment of the animal. It has been found that models based on the creation of transgenic mice containing this gene faithfully recapitulate the various features of the human LGFMS pathology. For example, this sequence may be operatively bound to a promoter that directs the expression of the sequence in Sca1⁺ cells, such as the mouse promoter pLy-6E.1 or a functional fragment or equivalent thereof, as discussed above.

The models generated according to the invention respond to drugs in a way that is predictive for the drug response in humans.

For example, a melphalan-prednisolone regimen which is unsuccessful in treating human multiple myeloma is also unsuccessful in the multiple myeloma models described herein. Doxorubicin, which is unsuccessful in treating human lung cancer is also unsuccessful in the lung cancer models described herein, for instance, in Sca1⁺hKRAS mice. The same is true for this drug in human and DLBCL modelled in mice according to the invention. Doxorubicin is one of the most actively used drugs in the treatment of human lymphomas. This is despite the reality that virtually all human lymphoma patients treated with doxorubicin relapse.

Gleevec™ (imatinib), which is unsuccessful in treating human Bcr Abl leukaemia is also unsuccessful in the BcrAbl leukaemia models described herein. Gleevec provides symptomatic relief in human CML, but again, does not cure CML in the mouse models of CML described herein. The clear hypothesis put forward by the inventors is that this failure to treat effectively is because Gleevec has not killed the CSCs. The models described herein can be used to develop a next generation Gleevec, which will kill CSCs and thus cure the cancer.

Furthermore, a successful treatment regime for human testis cancer is also successful in the mouse models described herein. In a model in which SNAIL is expressed in Sca1⁺ cells, mice treated with BEP (Bleomycin, VP16=etoposide, cis-platin) become tumour free within 7 weeks. BEP cures human testis carcinoma; it is highly toxic, but the only cancer treatment known to cure a cancer.

It is thus clear that the identification of CSCs represents a paradigm shift in the treatment of cancer. Data contained herein proves beyond doubt that CSCs are a real population of cells and responsible for generating cancer disease. However, before using CSC as targets in therapy programs (for target identification, drug discovery, etc.), it is important to demonstrate two things. First, that their ablation implies elimination of cancer in vivo following tumour formation in a whole animal. Secondly, it is necessary to know how the CSCs differ from normal stem cells, so that these can be selectively targeted.

Certainly, it is demonstrated herein that CSCs are able to maintain cancer disease. For example, an experiment disclosed herein shows that Sca1⁺Lin⁻ cells purified from the bone marrow of Sca1Bcl6 mice generate human B cell lymphomas in 100% of reconstituted mice. In contrast, spleen-derived tumor B-cells do not generate any lymphomas at all.

Additionally, the elimination of cancer following ablation of CSCs is demonstrated herein for the first time, using ganciclovir treatment of pLy6-HSVtk-IRES-BCRABLp210 mice (see Example 1). The HSVtk-IRES-BCRABLp210 construct is introduced into the Sca1⁺ gene under the operative control of the pLy6 promoter, in this instance. As expected given the expression pattern of BCRABLp210 constructs in the CML model described above, it was found that the pattern of BCR-ABLp210 and TK transcripts is high in Sca1⁺Lin⁻ cells and very low in Sca1⁻Lin⁺ cells. Expression of HSV-tk induces conversion of the prodrug nucleoside ganciclovir to its drug from as a phosphorylated base analogue. The phosphorylated ganciclovir is incorporated into the DNA of replicating cells causing irreversible arrest at the G2/M check point followed by apoptosis (Rubsam L Z et al. Cancer Res 1998, 58: 3873-3882). We developed this model to address the question as to whether ablation of Sca1+ cells can be used as a therapeutic target and to determine whether killing CSC might be an effective therapeutic strategy for cancer treatment. From a start point in which 100% of mice possessed tumours, 30 mice were used as a control and were subjected daily with saline. The 60 remaining mice were injected daily with GCV. During this period, the cancers disappeared completely. Furthermore, these mice carrying the cancers that disappeared following GCV treatment were observed for an additional 3 months in which no tumour recurrence was observed. Gancyclovir treatment in these mice thus leads to a complete reduction of tumour burden.

Exactly the same has been shown to be true of studies performed on mice containing an HSVtk-IRES-Bcl6 construct.

This work demonstrates for the first time that killing CSC is an effective therapeutic strategy for cancer treatment and its potential applications are broad.

The invention thus relates in part to purified CSCs. These CSCs have been isolated, purified and characterized. These purified CSCs may be isolated from a model according to any one of the aspects of the invention described above, particularly from a mouse model as described above. In an embodiment, the SC (such as a mSC) or the CSC (such as a mCSC) provided by the invention further comprises a marker, such as a marker useful for the specific isolation and/or identification of cancer stem cells, or for the procurement of cancer stem cells, or for the differentiation of cancer stem cells from healthy stem cells. Virtually any product from a SC or from a CSC capable of achieving said aims can be used, for example, genes, proteins, etc. Information concerning said markers can be used for designing assays for isolating and/or identifying SCs and/or CSCs, or for the procurement of SCs and/or CSCs, or for the differentiation of CSCs from healthy SCs. Due to the similarity between the mouse genome and the human genome, markers useful for the above mentioned aims may be also used for achieving the same aims in human SCs (hSCs) and/or human CSCs (hCSCs).

Accordingly, one aspect of the invention relates to a substantially pure culture of cancer stem cells. Preferably, said CSC is Sca1⁺. Such cells may express the Sca1 antigen at a significant level. Examples of other useful biomarkers include human epithelial antigen (HEA) in humans, CD133; α2β1 integrin. One, two, three, four, five, six or more markers of this type may be expressed by a stem cell according to the invention. The CSCs are preferably Lin−. The CSCs may also be CD38⁻.

The CSCs of the invention have the potential to propagate and maintain cancer. This ability may be tested using one or other of the assays mentioned herein. For example, transplantation of CSCs into healthy mice causes cancer, so giving a suitable functional validation assay. A specific example of a suitable assay is given in Example 13.

CSCs according to the invention may be any kind of cancer stem cells, such as, for example, prostate cancer stem cells, brain, breast, gut, colon, lung, ovarian, leukaemia, epithelial, solid tumour, or any mesenchymal and epithelial cancers. The CSCs may be any vertebrate cancer stem cells, but of greatest interest are animal stem cells, including both human and non-human cancer stem cells, mammalian, and rodent, including mouse cells in particular. Preferably, at least 50% of the cells in the substantially pure culture are cancer stem cells, and this proportion may be more, such as 60%, 70%, 80%, 90%, 95% or more. The substantially pure culture of cancer stem cells will preferably contain less than 50% of cancer cells that are not cancer stem cells, and this proportion may be less, for example, 25%, 10%, 5%, 1% or less.

Cancer stem cells according to the invention may be 5-fold enriched, 25-fold enriched, 50-fold enriched or more for CSCs relative to other types of cell, for example as compared to an untreated biological sample obtained directly from a patient or from a culture of cells. Such other cell types include non-stem cancer cells, non-cancerous stem cells and other, healthy cells that are present in the body or in in vitro culture.

Additionally, the cancer stem cells of the invention preferably do not express significant levels of one or more mature lineage markers selected from i) the group consisting of CD2, CD3, CD4, CD7, CD8, CD10, CD11b, CD14, CD15, CD16, CD19, CD20, CD31, CD45, CD56, CD64, CD140b and glycophorin A (GPA) in humans. The cancer stem cells of the invention preferably do not express CD24 or express low levels of CD24. The cancer stem cells of the invention are Lin− and do not express mature lineage markers.

By “significant levels” as used herein is preferably meant that the marker protein is expressed at a detectable level, for example, using antibodies in a blotting procedure, such as Western blotting.

According to particular aspects of the invention, various genes and their encoding proteins have been implicated in the CSC cancerous state. These discoveries allow a number of developments. For example, CSCs can be identified in an individual and relevant therapeutic intervention made, depending on the identity of these genes and proteins. Tumours can be stratified into groupings that are likely to undergo more favourable responses to cancer treatment. Specific evaluations can be made of a patient's expression profile for one or more of these genes, and based on these evaluations, a diagnosis can be formulated as to whether the patient is a suitable candidate for treatment, and if so, with what therapeutic agent. If so, a more detailed evaluation can then be made as to what form the treatment should best take (e.g. dosage, time and method of administration, drug combination).

For example, using microarrays, the inventors have discovered that the expression levels of Bmi-1, Nanog, PU.1, Pax-5, E2A, GATA3, c-Kit, CEBPg/z, R-IL3, Bcl2, Bid, Bak, PCDC2, p21, p53, mdm2, P27 and CCND2, differ in CSCs as compared to healthy SCs. Furthermore, the expression levels of these genes differ between CSCs before cancer onset and after cancer onset. This has been exemplified in lymphoma disease. Specifically, microarrays results analysis data show particularly significant differences in three markers: Bcl-2, mdm2 and GATA-3. The Bcl-2 marker presented a significant decrease in gene expression after lymphoma development compared to control levels. On the other hand, the last two markers, mdm2 and GATA-3, showed an increased response after lymphoma development compared to control. These results show that CSCs before cancer can be clinically detected are different from CSCs once cancer is clinically detected. These results indicate that CSC information can be used i) to predict cancer development in healthy humans, and ii) to prevent cancer development by prophylactic therapies, and so on. Such information cannot be obtained from studied CSCs obtained from cancer patients or conventional mouse models. The mouse models of the present invention thus represent a unique source to address the various challenges posed in this field.

This aspect of the invention thus provides a method of diagnosing an individual as susceptible to developing cancer or as suffering from cancer, the method comprising detecting the sequence, or level of expression or activity of any one of the genes from the group of Bmi-1, Nanog, PU.1, Pax-5, E2A, GATA3, c-Kit, CEBPg/z, R-IL3, Bcl2, Bid, Bak, PCDC2, p21, p53, mdm2, P27 and CCND2, or their expression products, in tissue from said patient and comparing said sequence, level of expression or activity to a reference, wherein a sequence, level of expression or activity that is different to said reference is indicative of disease or susceptibility to disease. Generally, for Bmi-1, PU.1, Pax-5a, CEBPg/z, Bcl2, Bid, Bak, PCDC2 and p21, a level that is significantly higher than the reference level will indicate that the individual is diseased or susceptible to disease. By “significant” is meant that the level of expression or activity is more than 10%, 25%, 50%, 100%, 250%, 500%, 1000% or more, higher than the reference level. Generally, for Nanog, E2A, GATA3, c-Kit, R-IL3, p53, mdm2, and CCND2, a level that is significantly lower than the reference level will indicate that the individual is diseased or susceptible to disease. The reference level is the level of expression in healthy stem cells from the patient.

In a further embodiment, the SC (such as an mSC) or the CSC (such as a mCSC) provided by the invention further comprises a reporter. A reporter, according to the instant invention, is a product which is attached to the surface of the cell membrane, or introduced inside the SC or CSC, which allows for spatiotemporal identification of the onset, progression, dissemination and further physiopathological processes as well as the effect of therapies by molecular imaging techniques. Illustrative, non-limitative molecular imaging techniques which can be applied include positron emission tomography (PET), computed tomography (TAC), nuclear magnetic resonance (NMR), high performance X-ray, etc., methods based on the emission of bioluminescent signals based on enzymatic reactions, methods based on the insertion of genetic constructions containing a label, such as green fluorescent protein (GFP), etc. Virtually any reporter capable of achieving said aims can be used. Said reporter can also play a role in diagnostic assays (e.g., molecular fingerprinting of a human disease, etc.), drug discovery and development processes, target identification as well as in improving the efficacy and reliability of all phases of the clinical development and its use in drug resistance or personalised medicine.

The CSCs of the invention may be isolated, enriched and purified in a sample, such as from a patient. The patient may be an individual diagnosed with cancer, an individual considered at risk from suffering cancer, an individual suspected of having cancer, or an individual not suspected of having cancer and who gives an outward impression of being in good health. The term “sample”, as used in the instant invention, can be any biological sample susceptible of containing SCs or CSCs, such as a liquid sample, for example, blood, serum, plasma, saliva, urine, etc., or a solid sample, such a tissue sample. Examples of suitable solid tissues include bone marrow, spleen, liver, lung, and so on. The sample can be obtained by any conventional method, including surgical resection in case of solid samples. The sample can be obtained from subjects previously diagnosed, or not diagnosed, with a human pathology of stem cell origin, e.g. cancer; or also from a subject undergoing treatment, or who has been previously treated for a said human pathology of stem cell origin.

This aspect of the invention allows CSCs to be isolated, enriched and purified from a patient or population of patients. This allows the development of assays to identify factors influencing cancerous growth in cancer stem cells, to analyse populations of cancer stem cells for patterns of gene expression or protein expression, to identify new anticancer drug targets, to predict the sensitivity of cancer stem cells to existing or new therapies, and generally to model cancer development and treatment.

In an alternative, the CSCs of the invention may be grown in culture. The cells may be isolated from an animal model, such as a mouse model of the type described herein. In addition to expression of Sca1, CSCs isolated from a cancer model animal may contain a chromosomal anomaly (herein described as an activatable gene) that is associated with cancer. Illustrative, non limitative examples of said chromosomal anomaly includes the genes identified as BCR-ABL^{p210}, BCR-ABL^{p190}, Slug, Snail, HOX11, RHOM2/LMO-2, TAL1, Maf-B, FGFR, c-maf, MMSET, BCL6, BCL10, MALT1, cyclin D1, cyclin D3, SCL, LMO1, LMO2, TEL-AML1, E2A-HLF, E2A-Pbx1, TEL-ABL, AML1-ETO, FUS-DDIT3, EWS-WT1, EWS FLI1, EWSR1-DDIT3, FUS-ATF1, FUS-BBF2H7, hKRASv12. The sequences of suitable activatable genes, including those mentioned above, are known from the prior art.

The invention also embraces methods of isolating CSCs. Such methods involve the selective enrichment of cancer stem cells which express the biomarkers of CSCs that are described herein. Suitable methods for the enrichment of cells expressing a marker include immunological-based systems such as fluorescence activated cell sorting, immunoaffinity exchange and so on. Other equivalent examples will be known to those of skill in the art and include density-adjusted cell sorting, magnetic cell sorting, antigen panning and so on.

The invention also relates to methods for propagating cancer stem cells of the invention. Such a method may involve exposing cancer stem cells in culture to a concentration of lysate produced from cells of at least one selected differentiated cell type, the concentration able to induce the cancer stem cells to propagate by preferentially undergoing either symmetric mitosis, whereby each dividing cancer stem cell produces two identical daughter cancer stem cells, or asymmetric mitosis, whereby each dividing cancer stem cell produces one identical daughter cancer stem cell and one daughter cell that is more differentiated than the cancer stem cells.

In a particular embodiment, the human pathology is a human pathology of stem cell origin. Examples of human pathologies have been mentioned previously. By way of illustration, the following genes, altered genes and gene fusions are implicated herein in particular human pathologies: Multiple Myeloma: maf-B, fgf-R, c-maf, MMSET; Lymphoproliferative Syndromes Bcl-6, Bcl-10, MALT1, CYC D1, CYC D3; T-cell acute leukaemia: SCL, Hox11, LMO2, LMO1; B-cell acute leukaemia: Bcr-ablp190, tel, E2A-HLF, E2a-Pbx1; Acute Myeloid Leukaemia: Tel-abl, AML1-ETO; Chronic Myeloid Leukaemia Bcr-ablp210; Liposarcomas: FUS-CHOP; Ewing Sarcomas: EWS-WT1, EWS-FLI1; and Lung Carcinomas: K-RasG12V (Cre recombinase inducible).

The SCs (including mSCs) and/or CSCs (including mCSCs) provided by the instant invention can be used as a biomarker in a number of strategies, such as, for example, for:
investigating and researching the cancer process; or for
identifying, classifying, isolating, purifying or describing CSC populations; or for
detecting the presence of a gene created and/or activated by a genetic anomaly associated with a human pathology in a subject; or for
assessing the risk or predisposition of a subject to develop a human pathology in a subject; or for
determining the stage or severity of a human pathology in a subject; or for
monitoring the effect of the therapy administered to a subject having a human pathology in a subject; or for
designing an individualized therapy for a subject suffering from a human pathology in a subject; or for
therapeutic monitoring and evaluation of therapeutic benefits; or for
designing human clinical trials and predicting the clinical outcome; or for
diagnosis of cancer and/or specific processes and effects of cancer development, like cancer dissemination; or for
patient selection for personalized therapeutics; or for
identifying drug repositioning with new and future drugs; or for
drug discovery and pharmacokinetics guidance; or for
previous/early cancer detection and predicting the likelihood of clinical relapse.

The use of the SCs (such as mSCs) and/or CSCs (such as mCSCs) provided by the instant invention as a biomarker for the above mentioned strategies constitutes an additional aspect of the instant invention.

Moreover, the SCs (such as mSCs) and/or CSCs (such as mCSCs) provided by the instant invention can also be used for discovering, screening, searching, identifying, evaluating and validating targets for human pathologies; or for identifying specific genes related to self-renewal ability of cancer stem cells. Advantageously, said genes related to (up or down) regulation of the self-renewal ability are specific only for cancer stem cells (not for healthy stem cells). The use of the SCs (such as mSCs) and/or CSCs (such as mCSCs) provided by the present invention for the previously above-mentioned purposes constitutes an additional aspect of the instant invention.

In another aspect, the invention relates to a method for detecting the presence of a gene created and/or activated by a genetic anomaly associated with a human pathology in a subject or for assessing the risk or predisposition of a subject to develop said pathology which comprises identifying a SC in a sample from said subject, said SC comprising a genetic anomaly associated with said human pathology. In a particular embodiment, said SC is a CSC. Accordingly, this aspect of the invention provides methods for diagnosing cancer. Such a method may comprise screening a subject for cancer, or a predisposition to cancer, comprising the steps of testing a sample from the subject for the presence of CSCs. A method of this type may include the steps of detecting cells expressing a product of a gene which is expressed in CSCs, examples of which are described herein. For example, the method may comprise the steps of: (a) contacting a ligand, such as an antibody against a CSC marker protein, with a biological sample under conditions suitable for the formation of a ligand-protein complex; and (b) detecting said complex. A level of expression on cells that is different to a control level is indicative of the presence of cancer stem cells. Such methods of diagnosis are advantageous over current methods of cancer diagnosis such as blood tests, and radiologic studies, which require relatively high numbers of cells. Accordingly, these diagnostic tests allow detection of a tumour at a much earlier stage than is possible now.

The expression product is preferably a protein, although alternatively mRNA expression products may also be detected. Where mRNA expression product is detected, it may, for example, be detected by the steps of contacting a tissue sample with a probe under stringent conditions that allow the formation of a hybrid complex between the mRNA and the probe; and detecting the formation of a complex.

Where mRNA expression product is used, it is preferably detected by the steps of contacting a tissue sample with a probe under stringent conditions that allow the formation of a hybrid complex between the mRNA and the probe; and detecting the formation of a complex. Preferred methods include comparing the amount of complex formed with that formed when a control tissue is used, wherein a difference in the amount of complex formed between the control and the sample indicates the presence of cancer. Preferably the difference between the amount of complex formed by the test tissue compared to the normal tissue is an increase or decrease. More preferably a two-fold difference in the amount of complex formed is deemed significant. Even more preferably, a 3-fold, 4-fold, 5-fold, 10-fold, 20-fold, 50-fold or even 100-fold increase or decrease in the amount of complex formed is significant.

In this alternative methodology, the method may comprise the steps of: a) contacting a sample of tissue from the patient with a nucleic acid probe under stringent conditions that allow the formation of a hybrid complex between a nucleic acid molecule coding for a CSC biomarker and the probe; b) contacting a reference sample with the probe under the same conditions used in step a); and c) detecting the presence of hybrid complexes in said samples; wherein detection of levels of the hybrid complex in the patient sample that differ from levels of the hybrid complex in the reference sample is indicative of disease or susceptibility to disease.

The method may comprise the steps of: a) contacting a sample of nucleic acid from tissue of the patient with a nucleic acid primer under stringent conditions that allow the formation of a hybrid complex between a nucleic acid molecule encoding a CSC biomarker and the primer; b) contacting a reference sample with the primer under the same conditions used in step a); c) amplifying the sampled nucleic acid; and d) detecting the level of amplified nucleic acid from both patient and reference samples; wherein detection of levels of the amplified nucleic acid in the patient sample that differ significantly from levels of the amplified nucleic acid in the reference sample is indicative of disease or susceptibility to disease relative to the reference state.

The method may comprise the steps of: a) obtaining a tissue sample from a patient being tested for disease; b) isolating a nucleic acid molecule encoding a CSC biomarker from the tissue sample; and c) diagnosing the patient by detecting the presence of a mutation which is associated with an altered susceptibility to cancer. This method may further comprise amplifying the nucleic acid molecule to form an amplified product and detecting the presence or absence of a mutation in the amplified product. The presence or absence of the mutation in the patient may be detected by contacting the nucleic acid molecule with a nucleic acid probe that hybridises to the nucleic acid molecule under stringent conditions to form a hybrid double-stranded molecule, the hybrid double-stranded molecule having an unhybridised portion of the nucleic acid probe strand at any portion corresponding to a mutation associated with the susceptibility profile; and detecting the presence or absence of an unhybridised portion of the probe strand as an indication of the presence or absence of a susceptibility-associated mutation.

The invention also includes ligands, such as antibodies, which bind specifically to, and which preferably inhibit the activity of a protein that is expressed on CSC cells. Such ligands may be used in the manufacture of a medicament for the diagnosis or therapy of a proliferative disease such as cancer or a disease or condition which involves a change in cell differentiation or growth rate.

The above methods can be performed in vitro or in vivo. Accordingly, the identification of said SCs or CSCs in a sample from the subject can be both diagnostic or prognostic of human pathologies in said subject. For example, the identification of said SCs or CSCs in a sample from the subject is indicative of human pathologies or a greater risk or predisposition of the subject to develop any of said pathologies.

Said SCs or said CSCs can be identified by conventional techniques in view of the markers and/or reporters to be identified, as described herein. In an embodiment, said cells can be identified by means of antibody-antigen cross-reactions, by means of ligand-receptor interactions, by molecular imaging techniques, etc. The above method can be performed in vitro or in vivo.

There are a wide variety of immunological assays available for detecting and quantifying the formation of specific antigen-antibody complexes; numerous competitive and non-competitive protein binding assays have previously been disclosed, and a large number of these assays are commercially-available.

Thus, when the marker or reporter to be detected acts as an antigen, said product can be quantified with antibodies. As used herein, the term “antibody” refers to intact molecules as well as to fragments thereof, such as Fab, F(ab′)2 and Fv, which are capable of binding, preferably binding specifically, to the antigenic determinant in question. The term “binds specifically” means that the antibodies have substantially greater affinity for their target polypeptide than their affinity for other related polypeptides. The antibodies may be specific for CSC associated proteins. For example, glycosylation patterns in cancer-associated proteins as expressed on CSCs may be different to the patterns of glycosylation in these same proteins as these are expressed on non-cancerous cells, such as healthy stem cells.

Preferably, antibodies are used which bind to the CSC target of interest with substantially greater affinity than they bind to other, non-related proteins. By “substantially greater affinity” we mean that there is a measurable increase in the affinity for the target polypeptide of the invention as compared with the affinity for other related polypeptides. Preferably, the affinity is at least 1.5-fold, 2-fold, 5-fold 10-fold, 100-fold, 10³-fold, 10⁴-fold, 10⁵-fold, 10⁶-fold or greater for the target polypeptide.

Preferably, the antibodies bind to the target with high affinity, preferably with a dissociation constant of 10⁻⁴M or less, preferably 10⁻⁷ M or less, most preferably 10⁻⁹ M or less; subnanomolar affinity (0.9, 0.8, 0.7, 0.6, 0.5, 0.4, 0.3, 0.2, 0.1 nM or even less) is preferred.

Monoclonal antibodies to target polypeptides of interest can be readily produced by one skilled in the art. The general methodology for making monoclonal antibodies using hybridoma technology is well known (see, for example, Kohler, G. and Milstein, C., Nature 256: 495-497 (1975); Kozbor et al., Immunology Today 4: 72 (1983); Cole et al., 77-96 in Monoclonal Antibodies and Cancer Therapy, Alan R. Liss, Inc. (1985). Other relevant texts include Goding, Monoclonal Antibodies: Principles and Practice, Academic Press, (1986) pp. 59-103; Waldmann, T. A. (1991) Science 252:1657-1662.

Chimeric antibodies, in which non-human variable regions are joined or fused to human constant regions (see, for example, Liu et al., Proc. Natl. Acad. Sci. USA, 84, 3439 (1987)), may also be of use.

The antibody may be modified to make it less immunogenic in an individual, for example by humanisation (see Jones et al., Nature, 321, 522 (1986); Verhoeyen et al., Science, 239, 1534 (1988); Kabat et al., J. Immunol., 147, 1709 (1991); Queen et al., Proc. Natl. Acad. Sci. USA, 86, 10029 (1989); Gorman et al., Proc. Natl Acad. Sci. USA, 88, 34181 (1991); and Hodgson et al., Bio/Technology, 9, 421 (1991)). The term “humanised antibody”, as used herein, refers to antibody molecules in which the CDR amino acids and selected other amino acids in the variable domains of the heavy and/or light chains of a non-human donor antibody have been substituted in place of the equivalent amino acids in a human antibody. The humanised antibody thus closely resembles a human antibody but has the binding ability of the donor antibody.

In a further alternative, the antibody may be a “bispecific” antibody, that is an antibody having two different antigen binding domains, each domain being directed against a different epitope. In the present case, one of the binding specificities may be for the target polypeptide, or a fragment thereof, the other one is for any other antigen, and preferably for a cell-surface protein or receptor or receptor subunit that is also expressed on cancer stem cells.

Antibodies used in the preset invention may therefore be monoclonal antibodies, polyclonal antibodies, intact or recombinant fragments thereof, “combibodies” and Fab or scFv antibody fragments, specific against said products; these antibodies being human, humanized or of non-human origin. The antibodies used in these assays can be labeled or not; the unlabeled antibodies can be used in agglutination assays; the labeled antibodies can be used in a wide variety of assays. Marker molecules which can be used to label antibodies include radionucleotides, enzymes, fluorophores, chemoluminescent reagents, enzyme substrates or cofactors, enzyme inhibitors, particles, colorants and derivatives.

There is a wide range of well known assays which can be used in the present invention using unlabeled antibodies (primary antibody) and labeled antibodies (secondary antibody); included among these techniques are the Western-blot or Western transfer, ELISA (Enzyme-Linked Immunosorbent Assay), RIA (Radioimmunoassay), competitive EIA (Competitive Enzyme Immunoassay), DAS-ELISA (Double Antibody Sandwich-ELISA), immunocytochemical or immunohistochemical techniques, techniques based on the use of biochips or microarrays of proteins including specific antibodies, or assays based on colloidal precipitation in formats such as dipsticks. Other ways to detect and quantify said products (markers or reporters) include affinity chromatography techniques, ligand binding assays and the like.

Cancer associated genes themselves may be detected by contacting a biological sample with a nucleic acid probe under stringent conditions that allow the formation of a hybrid complex between a nucleic acid expression product encoding a gene expressed in CSCs and the probe; and detecting the formation of a complex between the probe and the nucleic acid expression from the biological sample.

Preferred methods include comparing the amount of complex formed with that formed when a control tissue is used e.g. healthy stem cells, wherein a difference in the amount of complex formed between the control and the sample indicates the presence of cancer or a predisposition to cancer. Preferably the difference between the amount of complex formed by the test tissue compared to the normal tissue is an increase. More preferably a two-fold increase in the amount of complex formed is indicative of disease. Even more preferably, a 3-fold, 4-fold, 5-fold, 10-fold, 20-fold, 50-fold or even 100-fold increase in the amount of complex formed is indicative of disease.

In another aspect, the invention refers to a method for determining the stage or severity of a human pathology in a subject or monitoring the effect of the therapy administered to a subject having said pathology, which comprises identifying and quantifying the concentration of SCs in a sample from said subject, said SCs comprising a gene created and/or activated by a genetic anomaly associated with a human pathology, and comparing said concentration with that of a control sample or with a prior sample from said subject or with a prior sample from said subject taken before administering a therapy. In a particular embodiment, said SC is a CSC. Said SCs or said CSCs can be identified and quantified as previously mentioned by any suitable conventional technique. The above method can be performed in vitro or in vivo. This method farther comprises the step of comparing the concentration of SCs in a sample from said subject, said SCs comprising a gene created and/or activated by a genetic anomaly associated with a human pathology, with that of a control sample or with a prior sample from said subject or with a prior sample from said subject taken before administering a therapy. The control sample can be obtained from subjects free of human pathologies. According to this method, it is possible to determine the stage or severity of a human pathology in a subject or to monitor the effect of the therapy administered to a subject having said pathology.

The invention also provides methods for assessing the progression of cancer in a patient comprising comparing the expression products of genes expressed in CSCs in a biological sample at a first time point to the expression of the same expression product at a second time point, wherein an increase in expression, or in the rate of increase of expression, at the second time point relative to the first time point is indicative of the progression of the cancer.

In another aspect, the invention relates to a method for discovering, screening, searching, identifying, developing and/or evaluating compounds for treating a human pathology; or for repositioning known or future drugs or combinations of compounds, which comprises contacting a candidate compound with a SC (such as a mSC) or a CSC (such as a mCSC) provided by the instant invention, and monitoring the response. A compound which abolishes or slows down the proliferation of said cells or a compound which kills said cells can be selected for further assays. This method can be performed both in vitro or in vivo, for example, by xenografting SCs or CSCs into immunodeficient mice, etc.

Specifically, the invention provides assays for identifying a candidate agent that modulates the growth of a cancerous stem cell, comprising:

a) detecting the level of expression of an expression product of a gene expressed on CSCs in the presence of the candidate agent; and

b) comparing that level of expression with the level of expression in the absence of the candidate agent, wherein a reduction in expression indicates that the candidate agent modulates the level of expression of the expression product of the gene expressed in CSCs.

The invention also provides methods for identifying an agent that modifies the expression level of a gene expressed in CSCs, comprising:

a) contacting a cell expressing the gene with a candidate agent, and

b) determining the effect of the candidate agent on the cell, wherein a change in expression level indicates that the candidate agent is able to modulate expression.

Preferably the agent is a polynucleotide, a polypeptide, an antibody or a small organic molecule.

It is important to mention that the development of molecular and pharmacological therapeutics to treat and prevent human pathologies successfully, for example, pathologies of stem cell origin, such as cancer, will allow a precise assessment of the therapeutical potential of any strategy before the application in human therapy.

In another aspect, the invention provides a compound which abolishes or slows down the survival, division or proliferation, or induces differentiation of SCs or CSCs, e.g., cells of murine origin, or a compound which kills said cells. Said compound can be identified and evaluated according to any one of the above methods and is potentially useful for treating a human pathology. In an embodiment, said compound is selected or formed by:

a) an antibody, or combination of antibodies, specific against one or more epitopes present in said SCs or CSCs, e.g., mSCs or mCSCs; and

b) cytotoxic agents, such as antibiotics, toxins, compounds with radioactive atoms, or chemotherapeutic agents, which include, without limitation, small organic and inorganic molecules, peptides, phosphopeptides, antisense molecules, ribozymes, siRNAs, triple helix molecules, etc., which abolish or slow down the proliferation of said SCs or CSCs, e.g., mSCs or mCSCs, or kill said cells.

Examples of cytotoxic agents according to (b) include siRNA specific for CSC biomarkers.

Another aspect of the invention relates to the use of the above mentioned compound which abolishes or slows down the proliferation of SCs or CSCs, e.g., mSCs or mCSCs, or differentiates or kills said cells, in the manufacturing of a pharmaceutical composition for the treatment of a human pathology.

Specifically, the invention provides methods for the eradication of cancer stem cells. Such methods will include i) immunotherapy directed at cancer stem cells by targeting external or internal antigens or their coding genes; ii) inducing a switch in cancer stem cells either to effect a change from exponential to non-exponential cell growth or to induce a differentiation or apoptotic program. Such methods may target factors that are implicated in these changes, but should also be specific to CSCs.

The invention thus provides a method of treating a cancer disease in a patient in need of such treatment by administering to a patient a therapeutically effective amount of a protein, a nucleic acid molecule or ligand as described above. Such compounds may be administered in the form of a pharmaceutical composition. Such a composition will include the compound in conjunction with a pharmaceutically-acceptable carrier, as known to those of skill in the art.

The invention thus relates to treatments of cancer in a patient by ablating CSCs, for example, using compounds of the type described above. Such methods preferably reduce the number of CSCs. Such a method preferably comprises administering to the patient an antibody, a nucleic acid, a small molecule drug or a polypeptide in a therapeutically-effective amount sufficient to target cancer stem cells for destruction.

The identification herein of certain biomarkers that are selectively expressed on CSCs and not on healthy cells provides targets for therapies directed at selectively destroying the cancer stem cell compartment. Such therapies may target markers present on the cancer stem cells, either for the purpose of the destroying these cells, for example, by exploiting the existing immune system of the patient. This might be done, for example, by eliciting antibody-dependent cell cytotoxicity (ADCC). Alternatively, this might be done by inducing terminal differentiation, apoptosis or programmed cell death. This may be perhaps be effected by inducing a switch from a symmetric proliferative mitotic program to asymmetric cell division or a terminal differentiation/apoptotic program. By targeting the immortal population of cells within a tumour, these therapies are likely to be more successful than conventional therapies in reducing the number of cancer cells, and the patient is also less likely to suffer a relapse.

Thus, in another aspect, the invention relates to a pharmaceutical composition comprising a therapeutically effective amount of one or more compounds of those previously mentioned which abolish or slow down the proliferation of SCs or CSCs, e.g., mSCs or mCSCs, or differentiate or kill said cells, together with one or more excipients and/or carrier substances. Said pharmaceutical composition may also contain any other active ingredient useful for treating a human pathology. In a particular embodiment, said human pathology is a human pathology of stem cell origin.

The excipients, carrier substances and auxiliary substances must be pharmaceutically and pharmacologically tolerable, such that they can be combined with other components of the formulation or preparation and do not exercise adverse effects on the treated organism. Pharmaceutical compositions or formulations include those which are suitable for oral or parenteral administration (including subcutaneous, intradermal, intramuscular and intravenous), although the best administration route depends on the subject's condition. The formulations can be in single dose form. The formulations are prepared according to methods known in the field of pharmacology. The amounts of active substances to be administered can vary according to the particularities of therapy.

Another object of the invention consists in a kit for carrying out the present invention. Thus, an embodiment of the present invention provides a kit that comprises an antibody specific against a marker or a reporter eventually present in SCs or CSCs and a carrier in suitable packing, wherein said antibody is, for example, a monoclonal antibody, a polyclonal antibody, an intact or recombinant fragment thereof, a “combibody” or a Fab or scFv antibody fragment; said antibody being human, humanized or of non-human origin. Said antibody can be labeled or not; the unlabeled antibody can be used in agglutination assays; the labeled antibody can be used in a wide variety of assays. Marker molecules which can be used to label antibodies include radionucleotides, enzymes, fluorophores, chemoluminescent reagents, enzyme substrates or cofactors, enzyme inhibitors, particles, colorants and derivatives. The kit can also contain a combination of said antibodies. The kit may also contain a reagent useful for the detection of a binding reaction between said antibody and the marker or reporter polypeptide.

Furthermore, the invention provides kits useful for diagnosing cancer comprising a nucleic acid probe that hybridises under stringent conditions to a gene expressed in CSCs; primers useful for amplifying such a gene; and optionally instructions for using the probe and primers for facilitating the diagnosis of disease.

The kits of the invention can be employed, among other uses, for detecting the presence of SCs or CSCs, said cells containing a gene created and/or activated by a genetic anomaly associated with a human pathology of stem cell origin, or for assessing the risk or predisposition of a subject to develop a human pathology of stem cell origin in a subject; or for determining the stage or severity of a human pathology of stem cell origin in a subject; or for monitoring the effect of the therapy administered to a subject having a human pathology of stem cell origin (e.g., for therapeutic monitoring and evaluation of therapeutic benefits); or for designing an individualized therapy for a subject suffering from a human pathology of stem cell origin; or for designing human clinical trials; or for the diagnosis of cancer and/or specific processes and effects of cancer development, like cancer dissemination; or for patient selection for personalized therapeutics; or for drug discovery and pharmacokinetics guidance; or for discovering, screening, searching, identifying, evaluating and validating targets for human pathologies; or for identifying specific genes related to self-renewal ability of cancer stem cells.

In another aspect the invention refers to a method for designing an individualized therapy for a human suffering from a human pathology which comprises selecting a compound identified as previously mentioned, wherein said compound abolishes or slows down the proliferation of SCs or CSCs or kills said cells, said compound capable of being used as active principle in a pharmaceutical composition to be administered to said subject. This method can be performed both in vitro or in vivo.

In another aspect, the invention relates to a method for designing human clinical trials which comprises:
a) selecting targets for SCs (such as mSCs) or CSCs (such as mCSCs) provided by the instant invention or molecular profiling said cells;
b) validating said target or said molecular profile in a disease state; and
c) selecting responders vs non-responders for a particular treatment.

In another aspect, the invention provides a DNA construct, hereinafter the DNA construct of the invention, that comprises an activatable gene (i.e., a gene created and/or activated by a genetic anomaly associated with a human pathology) operatively bound to a promoter that directs the expression of said genetic anomaly in Sca1⁺ cells. Preferably, the genetic anomaly is selected from (i) a nucleic acid comprising an oncogene such as the BCL6 gene; (ii) a nucleic acid comprising a first nucleotide sequence coding for a kinase and a second nucleotide sequence comprising a genetic anomaly associated with a human pathology. Examples of such a genetic anomaly include BCR-ABL^{p210} and other examples of oncogenes that are described hereinabove; (iii) a nucleic acid coding for HSVtk-IRES-BCRABL^{p210}; (iv) a nucleic acid comprising a LMO2/RHOM2 gene sequence.

In the particular embodiment (iii) above, the DNA construct of the invention comprises a nucleic acid comprising a first nucleotide sequence coding for herpex simple thymidine kinase (HSV-tk), a second nucleotide sequence comprising BCR-ABL^{p210}, and a third nucleotide sequence comprising an internal ribosome-entry site (IRES) sequence, wherein 3′ end of said first nucleotide sequence is bound to the 5′ end of said third nucleotide sequence, and the 3′ end of said third nucleotide sequence is bound to the 5′ end of said second nucleotide sequence.

The DNA construct of the invention can be easily obtained by conventional digestion methods with restriction and binding enzymes, and similar enzymes as described by Sambrook, Fitsch and Maniatis, eds., (1989) “Molecular Cloning: A Laboratory Manual”. Cold Spring Harbor Laboratory Press, Cold Spring Harbor N.Y.

The DNA construct of the invention can be used, if desired, for the production of vectors useful for transforming mammal embryos and for generating transgenic animals using conventional methods such as those described by Sambrook et al., cited above.

Alternatively, the DNA construct of the invention can be used for obtaining a linear fragment of DNA useful for microinjection of DNA into fertilised oocytes in order to generate transgenic animals. Said linear fragment of DNA useful for microinjection can be obtained by means of cutting with restriction enzymes in order to obtain a linear DNA fragment that comprises the activatable gene.

In another aspect, the invention provides a transgenic non-human mammal that contains in its genome a DNA construct of the invention, in any one of the embodiments described above. The mammal may in certain embodiments contain more than one of these constructs, for example, two, three, four, five or more. The transgenic non-human mammal provided by this invention possesses, as a result, a genotype that confers a greater tendency to develop a human pathology such as cancer (e.g., of stem cell origin or, alternatively, of non stem cell origin) when compared to the non-transgenic mammal. Said non-human mammal is useful for studying the pathology among other goals and for evaluating potentially useful compounds for treating and/or preventing said pathology. Preferably, the human pathology is a solid tumour.

The expression “non-human mammal”, as is used in this description, includes any non-human animal, preferably belonging to the class of mammals, for example, rodents, and specifically mice. In preferred embodiments of the present invention as described above and below, the transgenic non-human animal and tissues or cells derived therefrom is preferably a mouse but may be another mammalian species, for example another rodent, for instance a rat, hamster or a guinea pig, or another species such as a chimpanzee, monkey, pig, rabbit, or a canine or feline, or an ungulate species such as ovine, caprine, equine, bovine, or a non-mammalian animal species. More preferably, the transgenic non-human animal or mammal and tissues or cells are derived from a rodent, more preferably, a mouse.

Although the use of transgenic animals poses questions of an ethical nature, the benefit to man from studies of the types described herein is considered vastly to outweigh any suffering that might be imposed in the creation and testing of transgenic animals. As will be evident to those of skill in the art, drug therapies require animal testing before clinical trials can commence in humans and under current regulations and with currently available model systems, animal testing cannot be dispensed with. Any new drug must be tested on at least two different species of live mammal, one of which must be a large non-rodent. Experts consider that new classes of drugs now in development that act in very specific ways in the body may lead to more animals being used in future years, and to the use of more primates. Accordingly, the benefit to man from transgenic models such as those described herein is not in any limited to mice, or to rodents generally, but encompasses other mammals including primates.

In a particular embodiment, the non-human transgenic animal provided by the invention are those described in the Examples accompanying the description.

For the generation of the transgenic non-human mammals provided by this invention, the DNA construct of the invention has been introduced into said mammal, or into a predecessor thereof, in an embryonic state, for example, in the state of a cell, or fertilized oocyte and, generally, not later than the g cell state.

Therefore, the invention provides a procedure for the preparation of a transgenic non-human mammal that possesses a genetic anomaly associated with a human pathology of stem cell origin, that comprises
(i) introducing a DNA construct of the invention into a fertilized oocyte of a non-human transgenic mammal;
(ii) implanting said fertilized oocyte into a pseudopregnant wet nursing mother to produce descendants; and
(iii) analysing said descendants to evaluate the existence of activated genes and/or genes created by a genetic anomaly associated with a human pathology of stem cell origin.

The method may comprise:

(i) introducing into a fertilised oocyte of a non-human transgenic mammal a DNA construct that comprises a gene created and/or activated by a genetic anomaly associated with development of a solid tumour operatively bound to a promoter that directs the expression of said gene in Sca-1+ cells;

(ii) implanting said fertilised oocyte in a pseudopregnant wet nursing mother to produce descendents; and

(iii) analysing said descendants.

In a particular embodiment, said genetic anomaly associated with a human pathology is a human pathology of stem cell origin, in which case, the descendents are analysed to evaluate the existence of activated genes and/or genes created by the genetic anomaly associated with the human pathology of stem cell origin in question.

There are different means conceived in the state of the art by which a sequence of nucleic acid can be introduced into an embryo of an animal such that it can be incorporated genetically in an active state, all of which can be applied to the generation of transgenic non-human mammals of the present invention. A method consists of transfecting the embryo with said sequence of nucleic acid as occurs naturally, and selecting the transgenic animals in which said sequence has been integrated onto the chromosome at a locus that gives as a result the activation of said sequence. Another method implies modification of the nucleic acid sequence, or its control sequences, before introducing it into the embryo. Another method consists of transfecting the embryo using a vector that contains the nucleic acid sequence to be introduced.

In a particular embodiment, the introduction of the DNA construct of the invention in the germ line of a non-human mammal is performed by means of microinjection of a linear DNA fragment that comprises the activatable gene operatively bound to the promoter that directs the expression in Sca1⁺ cells in fertilized oocytes of non-human mammals.

The fertilised oocytes can be isolated by conventional methods, for example, provoking the ovulation of the female, either in response to copulation with a male or by induction by treatment with the luteinising hormone. In general, a superovulation is induced in the females by hormonal action and they are crossed with males. After an appropriate period of time, the females are sacrificed to isolate the fertilised oocytes from their oviducts, which are kept in an appropriate culture medium. The fertilised oocytes can be recognised under the microscope by the presence of pronuclei. The microinjection of the linear DNA fragment is performed, advantageously, in the male pronucleus.

An alternative method for generation of animal models according to the invention comprises introduction of a DNA construct as mentioned above within an inactive locus of the mouse genome, for example, by homologous recombination through ES cells.

After the introduction of the linear DNA fragment that comprises the DNA construct of the invention in fertilised oocytes, they are incubated in vitro for an appropriate period of time or else they are reimplanted in pseudopregnant wet nursing mothers (obtained by making female copulate with sterile males). The implantation is performed by conventional methods, for example, anaesthetising the females and surgically inserting a sufficient number of embryos, for example, 10-20 embryos, in the oviducts of the pseudopregnant wet nursing mothers. Once gestation is over, some embryos will conclude the gestation and give rise to non-human transgenic mammals, which theoretically should carry the DNA construct of the invention integrated into their genome and present in all the cells of the organism. This progeny is the G0 generation and their individuals are the “transgenic founders”. The confirmation that an individual has incorporated the injected nuclear acid and is transgenic is obtained by analysing the individuals of the progeny. To do this, from a sample of animal material, for example, from a small sample from the animal's tail (in the event that it is, for example, a mouse) or a blood example, the DNA is extracted from each individual and analysed by conventional methods, for example, by polymerase chain reaction (PCR) using the specific initiators or by Southern blot or Northern blot analysis using, for example, a probe that is complementary to, at least, a part of the transgene, or else by Western blot analysis using an antibody to the protein coded by the transgene. Other methods for evaluating the presence of the transgene include, without limitation, appropriate biochemical assays, such as enzymatic and/or immunological assays, histological staining for particular markers, enzymatic activities, etc.

In general, in transgenic animals, the inserted transgene is transmitted as a Mendelian characteristic and so it is not difficult to establish the stable lines of each individual. If the G0 individuals are crossed with the parent strain (retrocrossing) and the transgene behaves with Mendelian characteristics, 50% of the progeny will be heterozygotic for the inserted transgene (hemizygotic). These individuals constitute the G1 progeny and a transgenic line that can be maintained indefinitely, crossing hemizygotics of the G1 generation with normal individuals. Alternatively, individuals of the G1 generation can be crossed among themselves to produce 25% homozygotics for the inserted transgene, 50% hemizygotics and 25% without the transgene provided the transgene does not affect the viability of the descendents.

The progeny of a non-human transgenic mammal provided by this invention, such as the progeny of a transgenic mouse provided by this invention can be obtained, therefore, by copulation of the transgenic animal with an appropriate individual, or by in vitro fertilization of eggs and/or sperm of the transgenic animals. As used in this description, the term “progeny” or “progeny of a non-human transgenic mammal” relates to all descendents of a previous generation of the non-human transgenic mammals originally transformed. The progeny can be analysed to detect the presence of the transgene by any of the aforementioned methods.

The invention also relates to a non-human transgenic mammal cell line that contains a DNA construct of the invention on its genome. In a particular embodiment, said cell line is a murine cell line.

The transgenic non-human mammal provided by this invention, its progeny or the cell line provided by this invention, are useful for, among other applications, evaluating potentially useful compounds for treating and/or preventing a genetic anomaly associated with neoplastic or non-neoplastic human pathology of stem cell origin or of non stem cell origin. Therefore, the invention also refers to the use of said non-human transgenic mammal, its progeny or a cell line provided by this invention, in the evaluation of potentially useful compounds for the treatment and/or prevention of a genetic anomaly associated with a human pathology. In a particular embodiment, said human pathology is a human pathology of stem cell origin.

In the case of transgenic animals, the evaluation of the potentially useful compound for the treatment and/or prevention of said human pathology of stem cell origin can be performed by administration of the compound to be tested to the transgenic animal, at different doses, and evaluating the physiological response of the animal over time. The administration of the compound to be assayed can be oral or parenteral, depending on the chemical nature of the compound to be evaluated. In some cases, it may be appropriate to administer the compound in question along with cofactors that enhance the effect of the compound.

In the case of cell lines of the invention, the evaluation of the potentially useful compound for the treatment and/or prevention of said human pathology of stem cell origin can be performed by adding the compound to be assayed to a cell culture medium for an appropriate period of time, at different concentrations, and evaluating the cellular response to the compound over time using appropriate biochemical and/or histological assays. At times, it may be necessary to add the compound in question to the cellular culture medium along with cofactors that enhance the effect of the compound.

The following examples illustrate the invention and should not be considered to limit the scope thereof. The practice of the present invention will employ, unless otherwise indicated, conventional techniques of molecular biology, microbiology, recombinant DNA technology and immunology, which are within the skill of those working in the art.

Most general molecular biology, microbiology recombinant DNA technology and immunological techniques can be found in Sambrook et al., Molecular Cloning, A Laboratory Manual (2001) Cold Harbor-Laboratory Press, Cold Spring Harbor, N.Y. or Ausubel et al., Current protocols in molecular biology (1990) John Wiley and Sons.

Unless defined otherwise, all technical and scientific terms used herein have the same meaning as commonly understood by one of ordinary skill in the art to which this invention belongs.

All documents cited herein are hereby incorporated by reference.

### BRIEF DESCRIPTION OF THE DRAWINGS

FIG. 1 shows transgene, thymidine kinase and BCR-ABLp210, expression in Sca1⁺Lin⁻ and Sca1⁻Lin⁺ lines.

FIG. 2 shows the percentage of mice developing tumours after ganciclovir treatment and saline solution treatment (control).

FIG. 3 shows three representative pictures of (a) normal spleen, (b) a spleen which has developed DLBCL, (c) a spleen which has developed a less aggressive Burkitt lymphoma and (d) a spleen which has developed a more aggressive Burkitt lymphoma.

FIG. 4 is a graphical description of expression data for the known target genes in the bone marrow cancer stem cell of the Sca1⁺Bcl6 mice. Each gene (identified at right) is represented by a single row of coloured boxes; each mouse is represented by a single column.

FIG. 5 is a graphical description of the expression pattern in the Bcl6 mouse model of some of the described genes up/down regulated in the human Diffuse large b-cell Lymphoma (DLBCL).

FIG. 6 provides a schematic representation of the SCA1/BCR-ABL p210 gene insertion into SCA1 used in the construction of the CML mouse model as well as a graph summarising the measurement of BCR-ABL p210 transcript levels in Sca+Lin− and Sca−Lin+ cells, before and after CML.

FIG. 7 shows that SCA1/BCR-ABL p210 CSC mice reproduce the pathology of human CML: Megakaryocytes define myeloid metaplasia in the spleen and liver and mature myeloid cells accumulate in peripheral blood.

FIG. 8 is a representation of chromosome staining light microscopic images to show that SCA1/BCR-ABL p210 CSC mice exhibit DNA methylation, which is associated with malignant transformation and is also found in human CML.

FIG. 9 shows that SCA1/BCR-ABL p210 CSC mice progress spontaneously from CML to blast crisis, reproducing the pathology of human CML. Analysis by flow cytometry shows that blast cells, but no mature granulocytes are present in peripheral blood. Histochemical analysis shows that blast cells infiltrate both liver and peripheral blood.

FIG. 10 shows that SCA1/BCR-ABL p210 CSC mice reproduce the fibrosis of spleen and liver seen in human CML (green staining of liver and spleen sections in Masson's trichrome staining protocol).

FIG. 11 shows that SCA1/BCR-ABL p190 CSC mice reproduce the pathology of human B-ALL. Flow cytometric results and/or histochemical analysis on blood smears and spleen, liver, and lung samples show extensive B cell blast infiltration of peripheral blood, spleen, liver, and lung.

FIG. 12 shows that SCA1/LMO2-RHOM2 CSC mice reproduce the pathology of human T-ALL (flow cytometric data and histochemical data of the spleen, of thymoma, liver, kidney and testes). The development of T-cell leukemia and blast dissemination into tissues and peripheral blood are identical to the human pathology.

FIG. 13 shows that SCA1/Maf-B CSC mice reproduce the pathology of human multiple myeloma (histochemical data). Plasma cells infiltrate the spleen and kidney, with pathological indications of kidney failure.

FIG. 14 shows that SCA1/Maf-B CSC mice reproduce the pathology of human multiple myeloma. Flow cytometric and histochemical analyses reveal plasma cells in bone marrow and peripheral blood, as in human multiple myeloma.

FIG. 15 summarises experiments showing that SCA1/BCL6 CSC mice reproduce the pathology of human lymphoma. A schematic representation of the BCL6 insertion into the 5′-UTR of the SCA1 gene is provided. The C57BL/6×CBA mice used to construct the SCA1/BCL6 mouse model of lymphoma developed lymphoma after 5-7 months. Measurement of BCL6 transcript levels shows that BCL6 is transcribed in Sca1+Lin− cells, but not in Sca1−Lin+ cells.

FIG. 16 shows that SCA1/BCL6 CSC mice reproduce the pathology of human lymphoma (histochemical analysis of spleen tissue). The diffuse large B cell lymphoma (BLBCL) found in the spleen of this CSC mouse model mimics the DLBCL seen in man.

FIG. 17 shows that SCA1/BCL6 CSC mice reproduce the pathology of human lymphoma. Histochemical analysis of spleen, lung and kidney tissue reveals (1) that Pax5+ and CD21+ B cells infiltrate lung and kidney tissue and (2) that B cells do not express BCL6.

FIG. 18 summarises the observation that, similarly to human B cell lymphomas, SCA1/BCL6 CSC mice do not express BCL6 in B cells. The BCL6−/− phenotype is not rescued in SCA1/BCL6×BCL6−/− mice.

FIG. 19 summarises FACS and hybridisation analysis results showing that SCA1/BCL6 CSC mice exhibit a similar pathology to human lymphoma also on the molecular level. Hybridisation experiments with isolated CD22+B220+ cells show that molecular prognostic markers, such as those listed in the figure which are used to classify human DBCL, are present in the CSC cancer mouse model.

FIG. 20 summarises the observation that SCA1/BCL6 CSC mice are similar to human “activated B cell-like” (ABC) DLBCL rather than “germinal centre B cell-like” (GCB) DLBCL. Just as in the case of CD19+ CD22+ cells in human ABC DLBCL, B220+ CD22+ cells in the SCA1/BCL6 CSC DLBCL mouse model do not express BCL6.

FIG. 21 further summarises the similarity between SCA1/BCL6 CSC mice and human “activated B cell-like” (ABC) DLBCL in terms of regulation and levels of gene expression and associated cell differentiation. Sca1-BCL6 mice exhibit a plasma cell differentiation blockade similar to that observed in human ABC DLBCL.

FIG. 22 provides a schematic representation of the loxP-Stop-loxP-hK-Ras^{v12} construct used in a lung carcinoma model, as well as results of histochemical analysis showing that, in this model, oncogenic activation in solid tissue Sca1+ cells leads to solid tissue carcinomas in mice. Non-small cell lung cancer (NSCLC) lung carcinoma were observed 3-5 months after Adeno-Cre infection.

FIG. 23 summarises the development of solid tumours in the CSC mouse model of CML.

FIG. 24 summarises the development of solid tumours in the SCA1/RHOMB2 CSC mouse model of T-ALL.

FIG. 25 summarises the development of solid tumours in the CSC mouse model of B-cell lymphoma.

FIG. 26 summarises the development of solid tumours in the SCA1/HOX11 CSC mouse model of T-ALL.

FIG. 27 shows the result of crossing mice carrying the Cre recombinase in the 5′-UTR of the Sca1 gene with the ROSA26 reporter strain, in which Bgal activity/expression is prevented by a stop cassette “floxed” by loxP sites. Sections of skin and testis of the Rosa26×Sca1-CRE mice thus generated were subjected to B-gal staining. Regions stained blue were those regions in which stem cells are normally located A skin sample of the ROSA26×SCA1/CRE progeny is shown.

FIG. 28 shows results from the same experiment as FIG. 27 relates to, except that a testis sample of the ROSA26×SCA1/CRE progeny is shown.

FIG. 29 provides a schematic representation of the HVTK-IRES-BCR-ABL p210 gene insertion into SCA1 used in the construction of the CML mouse model, as well as a graph summarising the measurement of TK and BCR-ABL p210 transcript levels in Sca+Lin− and Sca−Lin+ cells. This was one of the CSC mouse models used to prove that CSC ablation is an effective treatment strategy for cancer.

FIG. 30 shows that cancer in a SCA1/BCL6 CSC mouse cancer model may be effectively treated by killing reprogrammed stem cells (CSC). No tumours were detectable 10 weeks after the beginning of therapy and no tumours were detectable during 11 further weeks of observation.

FIG. 31 shows that cancer in a SCA1/BCR-ABL p210 CSC mouse cancer model may be effectively treated by killing reprogrammed stem cells (CSC). No tumours were detectable 8 weeks after the beginning of therapy and no tumours were detectable during 8 further weeks of observation.

FIG. 32 provides an overview of the “floxed” BCR-ABL p210/GFP construct. Floxed means BCR-ABL p210 is flanked by loxp loci. The BCR-ABL p210 in this construct—or any other gene floxed in the same manner—may thus be inactivated by means of Cre-mediated loxP mutagenesis. GFP is located downstream of and/or adjacent to floxed BCR-ABL p210. This allows expression of GFP when the floxed gene—BCR-ABL p210 in this figure—is inactivated by means of Cre-mediated loxP mutagenesis. This construct was used to test whether BCR-ABL p210 is required for tumour malignancy. Equivalent constructs containing other oncogenes in the place of BCR-ABL p210 allow testing of whether said other oncogenes are required for tumour malignancy.

FIG. 33 summarises the experiment testing the relationship between BCR-ABL p210 and tumour malignancy, as described in Example 14.

FIG. 34 summarises the observation that CSC-driven cancers in the SCA1/BCR-ABL p210 CSC model and in human CML in the clinic respond to treatment with Gleevec™ in a similar manner. The ABL tyrosine kinase inhibitor Imanitib/Gleevec™ does not kill CSC in mice.

FIG. 35 is a graph depicting the results of treatment of BCR-ABL p210 bone-marrow recipient mice with Gleevec™. The treatment was ineffective, as is described in Example 15.

FIG. 36 is a graph showing unsuccessful treatment of BCR-ABL p190 leukemia with Gleevec™ in cancer stem cell model mice. The treatment was ineffective, as is described in Example 15

FIG. 37 shows that, as in humans, treatment of diffuse large B-cell lymphoma with doxorubicin is ineffective in CSC mouse models (cell counting data; cf. Example 16).

FIG. 38 shows that, as in humans, treatment of diffuse large B-cell lymphoma with doxorubicin is ineffective in the Sca1/hKRAS CSC mouse model (survival study; cf. Example 16).

FIG. 39 shows that, as in humans, treatment of multiple myeloma with melphalan prednisolone is ineffective in the Sca1/MAF-B CSC mouse model (survival study; cf. Example 17).

FIG. 40 shows that the successful BEP treatment regime for human testicular cancer is also successful in the corresponding SCA1/SNAIL CSC mouse model. Mice treated with BEP (Bleomycin, etoposide/VP16, cis-platin) became tumour-free within 7 weeks (cf. Example 18).

### MATERIALS AND METHODS

Production of Transgenic PLy6-HSVtk-IRES-BCRABLp210 Mice

For the generation of the transgenic non-human mammal provided by this invention, DNA constructs, such as HSVtk-IRES-BCRABLp210, were introduced into said mammal, or into a predecessor thereof, in an embryonic state, for example, in the state of a cell, or fertilized oocyte and, generally, not later than the g cell state.

The procedure for the preparation of a transgenic non-human mammal that possesses this chromosomal anomaly associated with a human pathology of stem cell origin, is as follows:
(i) introducing a DNA construct of the invention into a fertilized oocyte of a non-human transgenic mammal;
(ii) implanting said fertilized oocyte into a pseudopregnant wet nursing mother to produce descendants; and
(iii) analysing said descendants to evaluate the existence of activated genes and/or genes created by a chromosomal anomaly associated with a human pathology of stem cell origin.

Ganciclovir (GVC) Treatment

PLy6-HSVtk-IRES-BCRABLp210 mice were used to test the effectiveness of GCV-induced cell death in CSC that express BCR-ABL. GCV, after preliminary testing, was administered at a dose of 100 mg/kg/day by i.p. injection for 14 days. This dose has been reported to kill cells expressing HSV-tk in transgenic mice (Bush TG. Cell 1998, 93: 189-201). Dosing started when the mice were leukaemic. A control group was given injections of normal saline.

RNA Extraction

Total RNA was isolated in two steps using TRIzol (Life Technologies, Inc., Grand Island, N.Y.) followed by Rneasy Mini-Kit (Qiagen Inc., Valencia, Calif.) purification following the manufacturer's RNA Clean-up protocol with the optional On-column Dnase treatment. The integrity and the quality of RNA were verified by electrophoresis and its concentration was measured.

Target Preparation

T-7-based RNA amplifications and preparations of cDNA probes were performed as described previously (Van Gelder R N, von Zastrow M E, Yool A, Dement W C, Barchas J D, Eberwine J H: Amplified RNA synthesized from limited quantities of heterogeneous cDNA. Proc Natl Acad Sci USA 1990, 87:1663-1667. Eberwine J: Amplification of mRNA populations using aRNA generated from immobilized oligo(dT)-T7 primed cDNA. Biotechniques 1996, 20:584-591). Briefly, a maximum amount of 5 μg of total RNA were converted to double-stranded cDNA using the superscript choice system (Life Technologies) using oligo-dT primer containing a T7 RNA polymerase promoter. The double-stranded cDNA was cleaned up, and T7 in vitro transcription was performed using Megascript T7 in vitro transcription kit (Ambion, Austin, Tex.) following the manufacturers' instructions.

Microarray Procedure

2.5 μg of second round amplified RNA from each sample was directly labeled with cyanine 3-conjugated dUTP (Cy3), whereas 2.5 μg of second round amplified RNA from the Universal Mouse Reference RNA (Stratagene) was labeled with cyanine 5-conjugated dUTP (Cy5) as reference. For all microarray studies a mouse cDNA array was used. Hybridizations were performed as described. After washing, the slides were scanned using a Scanarray 5000 XL (GSI Lumonics Kanata, Ontario, Canada) and images were analyzed with the GenePix 4.0 program (Axon Instruments Inc., Union City, Calif.).

Data Analysis

Data obtained from each hybridization were stored in a database for analysis. The Cy3:Cy5 ratios were normalized to the median ratio value of all of the spots in the array. After normalization, spots with intensities for both channels (sum of medians) less than that of the local background were discarded. The ratios of the remaining spots were log transformed (base 2), and duplicated spots on the mouse chip were averaged to the median. To obtain the expression profile of cancer stem cells (CSC), we referred the ratios of the CSC to the control hematopoietic stem cells. Genes were selected to be upregulated or down-regulated if the difference in ratio was at least 2-fold. For clustering analysis, the SOTA clustering program (Yuspa S H et al. Cancer Res 1980; 40:4694-703) was used, and trees were viewed using the TreeView program.

Flow Cytometry

Nucleated cells were prepared from peripheral blood cell suspensions. In order to further prepare cells for flow cytometry, contaminating red blood cells were lysed with 8.3% ammonium chloride and the remaining cells were then washed in PBS with 2% foetal calf serum (FCS). After staining, all cells were washed once in PBS with 2% FCS containing 2 μg/mL propidium iodide (PI) to allow dead cells to be excluded from both analyses and sorting procedures. Monoclonal antibodies were obtained from Pharmingen and included: antibodies against CD45R/B220, CD19, Ly51, CD43, IgM and IgD for B lineage staining; antibodies against CD4, CD8 and CD3 for T cell lineage; antibodies against CD11b and Gr1 for myeloid lineage and Sca1 for stem cell staining Single cell suspensions from the different tissue samples obtained by routine techniques were incubated with purified anti-mouse CD32/CD16 (Pharmingen) to block binding via Fc receptors and with an appropriate dilution of the different antibodies at room temperature or 4° C., respectively. The samples and the data were analysed in a FACScan apparatus using CellQuest software (Becton Dickinson, FACS-sorter apparatus). The specific fluorescence of fluorescein isothiocyanate (FITC) and PE (phycoerythrin) was excited at 488 nm (0.4 W) and 633 nm (30 mW), respectively. Known forward and orthogonal light scattering properties of mouse cells were used with established gates. For each analysis, at least 5.000 viable (PI⁻) cells were assessed.

Treatment of Animals with Anticancer Drugs

Treatment of animals with anticancer drugs was carried out as follows:

BEP (Bleomycin, etoposide/VP16, cis-platin): For the treatment: of testis carcinoma by the BEP protocol, bleomicine was administered at 3 mg/day (days 1-5), VP16 at 100 mg/m² (days 1-5) and cis-platin at 40 mg/m² (days 1-5) for 3 weeks. Water was used as placebo. Drugs were dissolved in water and delivered intravenously.

DXR (doxorubicin) was dosed at 7 mg/kg. The administration scheme was every 7^{th} day, 3 times—q7dx3—for a duration of 4-5 weeks. Drugs were dissolved in water and delivered intravenously. Water was used as placebo.

Gleevec™: Stock solutions of 5 mg/ml and 10 mg/ml were made freshly in water, sterile filtered and administered to mice in a volume of 250 ml by gavage twice a day. The Gleevec™ regimen was 50 mg/kg every morning and 100 mg/kg every evening by gavage. Gleevec™ was administered in a volume of 250 microliter of sterile water. Water was used as placebo.

Ganciclovir: stock solutions were prepared following the SIGMA indications. The regimen was 100 mg/kg/day by intraperitoneal injection for 14 days. The placebo was an equal volume of diluent water/normal saline.

Melphalan and prednisone: for two weeks, melphalan (2.5 mg/kg/day) was delivered intravenously and prednisolone (10 mg/kg/day-) was delivered in daily oral doses. The placebo was an equal volume of PBS.

Real-Time PCR Quantification

To quantify expression levels, reverse transcription (RT) was performed according to the manufacturer's protocol in a 20-μl reaction containing 50 ng of random hexamers, 3 μg of total RNA, and 200 units of Superscript II RNase H-free (RNase H⁻) reverse transcriptase (GIBCO BRL). Real-time quantitative PCR was carried out for the quantitation of BCR-ABL p210. Fluorogenic PCRs were set up in a reaction volume of 50 μl using the TaqMan PCR Core Reagent kit (PE Biosystems). cDNA amplifications were carried out using the same primers in a 96-well reaction plate format in a PE Applied Biosystems 5700 Sequence Detector. Thermal cycling was initiated with a first denaturation step of 10 min at 95° C. The subsequent thermal profile was 40 cycles of 95° C. for 15 s, 56° C. for 30 s, 72° C. for 1 min. Multiple negative water blanks were tested and a calibration curve determined in parallel with each analysis. The Abl endogenous control (PE Biosystems) was included to relate BCR-ABL p210 to total cDNA in each sample.

Sequences of specific primers and probe were as follows:

BCR-ABL^{p210}, sense primer
5′-TTCTGAATGTCATCGTCCACTCA-3′,
antisense primer
5′-AGATGCTACTGGCCGCTGA-3′
and
probe
5′-CCACTGGATTTAAGCAGAGTTCAAAAGCCC-3′;
c-Abl, sense primer
5′-CACTCTCAGCATCACTAAAGGTGAA-3′,
antisense primer
5′-CGTTTGGGCTTCACACCATT-3′,
and
probe
5′-CCGGGTCTTGGGTTATAATCACAATG-3′.

### EXAMPLES

### Example 1

### Murine CSC after Treatment with Ganciclovir (GCV)

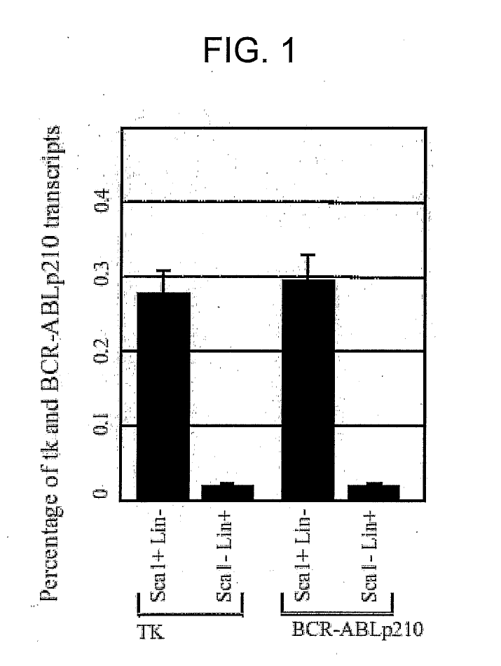

In PLy6-HSVtk-IRES-BCRABLp210 mice (see under Materials and methods), transgene expression, thymidine kinase and BCR-ABLp210, in Sca1⁺Lin⁻ and Sca1⁻Lin⁺ lines were studied. Results showed high level of expression of both transgenes only in the Sca1⁺Lin⁻ lines (see FIG. 1). This expression pattern confirmed a selective transgene expression in the cancer stem cells (Sca1⁺Lin⁻).

In Table 1 the different cancer types developed in PLy6-HSVtk-IRES-BCRABLp210 mice are represented. The results demonstrate that said transgenic mice developed the same tumour pattern as the one observed in humans affected with the BCRABLp210 genetic anomaly.

TABLE 1
Cancer development pattern in PLy6-HSVtk-IRES-BCRABLp210
mice

| | |
|---|---|
| CML only | 81% |
| CML + Hepatic ADC | 3% |
| CML + Fibrohistiocytoma | 2% |
| CML + Sarcoma osteogenic | 2% |
| CML + Tumour cell. Sertoli | 2% |
| CML + Lung ADC | 10% |

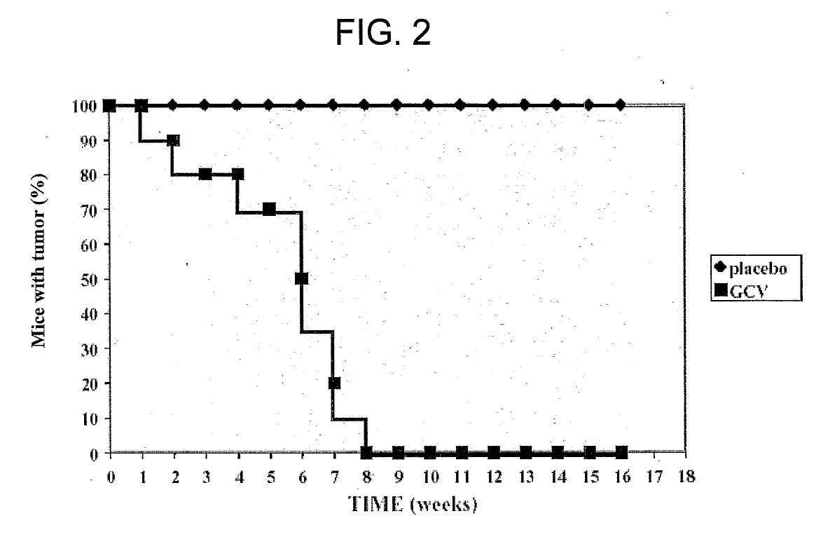

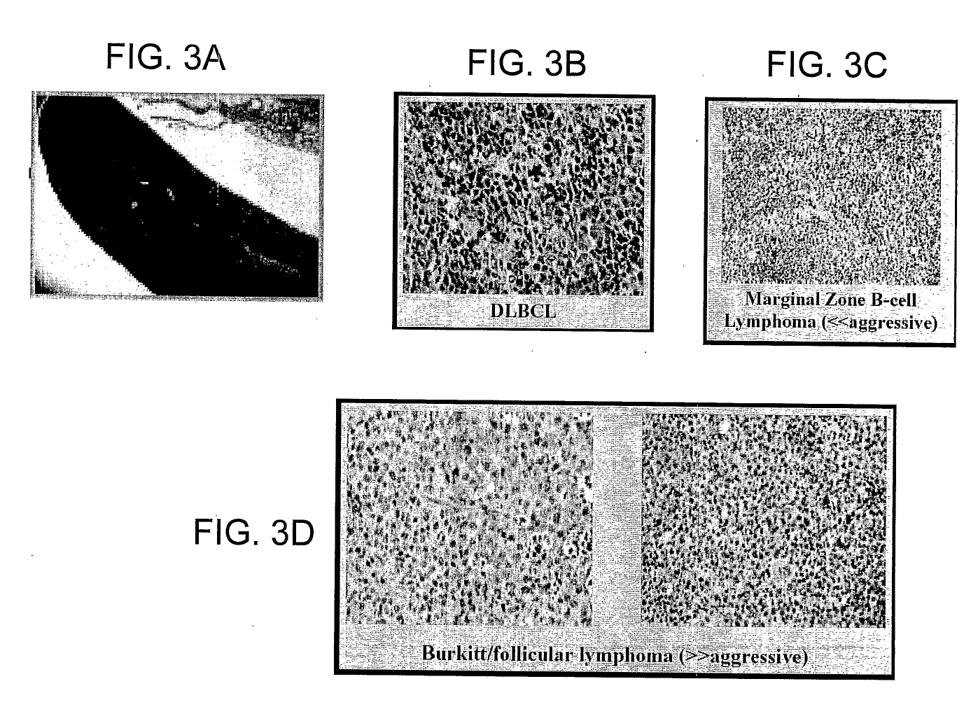

30 mice were used as a control and were subjected daily with saline solution whereas the 60 remaining mice were injected daily with GCV as described in method section. During this period, cancers disappeared completely. The mice from which cancers had disappeared following treatment were observed for an additional 3 months in which no tumour recurrence was observed (CML and solid tumours) (FIG. 2).

### Example 2

### Genetic Profile of Murine CSC

Microarray analysis revealed a different expression profile of cancer stem cell before and after lymphoma development. These data show significant differences in three markers: Bcl-2, mdm2 and GATA-3. Bcl-2 marker presented a significant decrease in gene expression after lymphoma development compared to control levels. On the other hand, the last two markers, mdm2 and GATA-3, showed an increased response after lymphoma development compared to control (see Table 2).

TABLE 2
CSC (Sca1+/Lin−)

Lymphoma development

| | Before | After |
|---|---|---|
| Bmi-1 | + | |
| Nanog | − | |
| PU.1 | + | + |
| Pax-5 | + | +/− |
| E2A | − | |
| GATA3 | − | + |
| c-Kit | − | |
| CEBPg/z | + | + |
| R-IL3 | − | |
| Bcl2 | + | − |
| Bid | + | + |
| Bak | + | +/− |
| PCDC2 | + | +/− |
| p21 | + | |
| p53 | − | |
| mdm2 | − | + |
| P27 | | |
| CCND2 | − | |

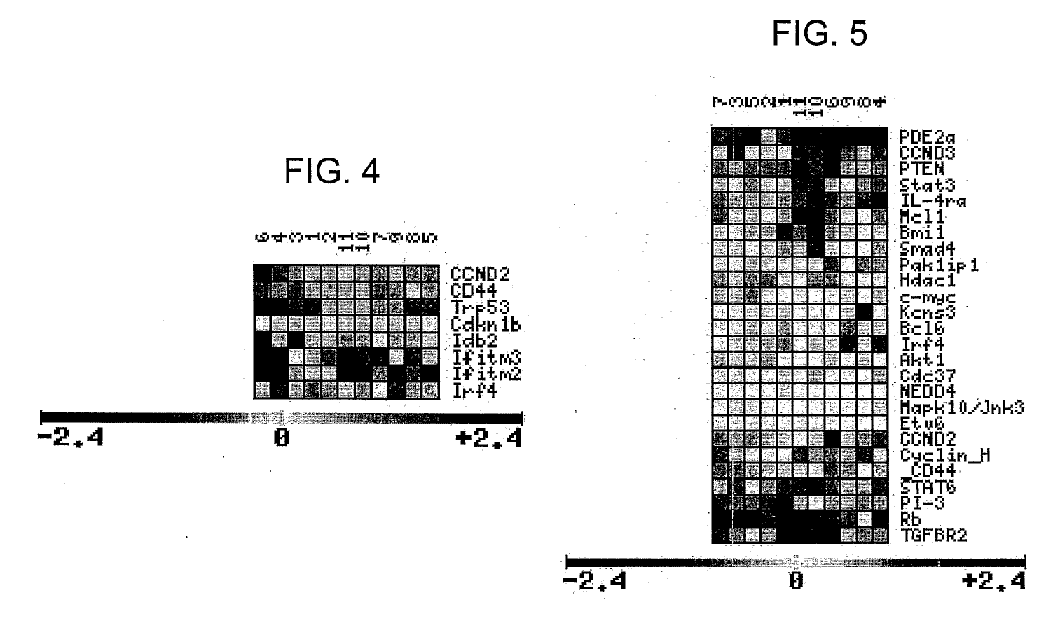

FIG. 4 shows a gene expression pattern corresponding to cancer stem cells from Bcl6 mice (Sca1⁺Lin⁻). On the order hand, the murine cancer stem cells harboring BCL6 transgene show a similar expression profile (FIG. 5) to the lymphoma human B-cells (Alizadeh A A, Eisen M B, Davis R E, Ma C, Lossos I S, Rosenwald A, Boldrick J C, Sabet H, Tran T, Yu X, Powell J I, Yang L, Marti G E, Moore T, Hudson J Jr, Lu L, Lewis D B, Tibshirani R, Sherlock G, Chan W C, Greiner T C, Weisenburger D D, Armitage J O, Warnke R, Levy R, Wilson W, Grever M R, Byrd J C, Botstein D, Brown P O, Staudt L M. 2000. Distinct types of diffuse large B-cell lymphoma identified by gene expression profiling. Nature. February 3; 403(6769): 503-11). This shows that the murine cancer stem cells harbouring the Bcl6 transgene can be used as a model to mimic human lymphoma.

### Example 3

### Creation of CSC-Based Mouse Cancer Models

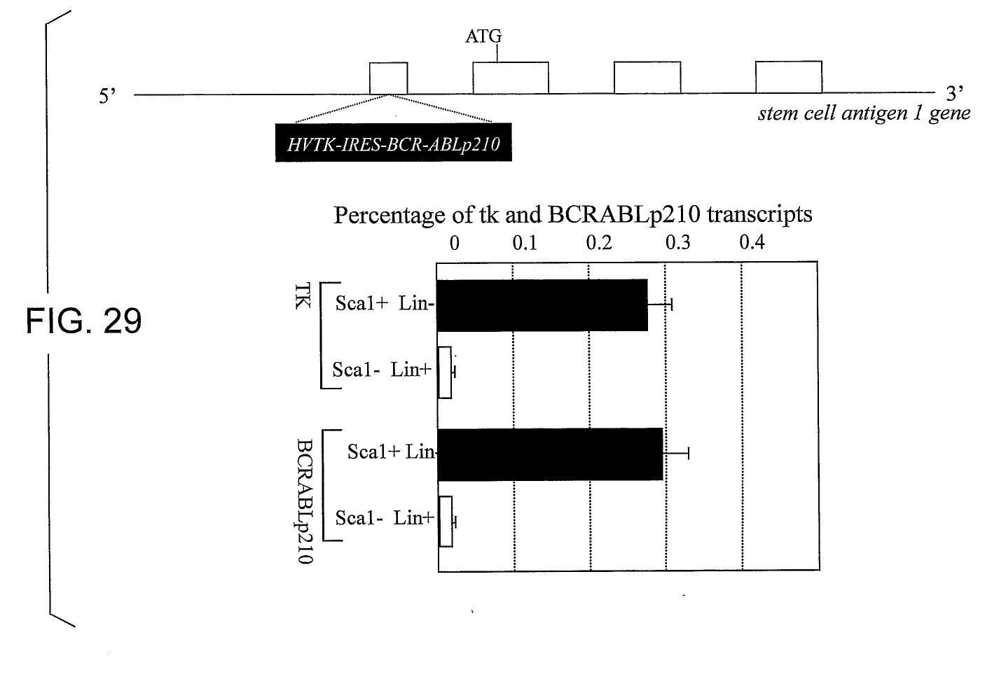

The inventors developed numerous further mouse models of cancer in which oncogenes (both human genes or their mouse counterparts) were inserted into the 5′-untranslated region (5′-UTR) of the Sca1 (stem cell antigen 1) gene (FIG. 29). As is set out in further detail in the examples below, these mouse models were subsequently used to address the question of whether the specific killing of CSC might be an effective therapeutic strategy for cancer treatment, as well as other, related questions.

Table 3 lists some of the human cancers for which mouse models were obtained.

TABLE 3
CSC mouse models and associated transgenic oncogenes/chromosomal
anomalies incorporated into SC to obtain CSC

| Type of cancer | Oncogene |
|---|---|
| Multiple myeloma | Maf-B, FGF-R, c-maf, |
| | MMSET |
| Lymphoproliferative syndromes | BCL-6, BCL-10, MALT1, |
| | CYC D1, CYC D3 |
| T-cell acute leukemia (T-ALL) | SCL, HOX11, LMO1, |
| | LMO2-RHOM2 |
| B-cell acute leukemia (B-ALL) | BCR-ABL^{p190}, TEL-AML1, |
| | E2A-HLF, E2A-Pbx1 |
| Acute myeloid leukemia | TEL-ABL, AML1-ETO |
| Chronic myeloid leukaemia (CML) | BCR-ABL^{p210} |
| Liposarcomas | FUS-CHOP |
| Ewing sarcomas | EWS-WT1, EWS-FLI1 |
| Lung carciomas | K-Ras^{G12V} |
| | (Cre recombinase inducible) |
| Diffuse large B-cell lymphoma | BCL6 |

Constructs according to the present invention that were used in the construction of the mouse models according to the invention included the HVTK-IRES-BCR-ABL p210 as shown in FIG. 29 or, e.g., the equivalent constructs for BCL6 (BCL6-IRES-HVTK) or hKRAS, and constructs for the other oncogenes listed in Table 3.

HVTK is an alternative abbreviation for HSV-tk, where HSV stands for Herpes simplex virus and tk (or TK) stands for thymidine kinase. IRES is an abbreviation for internal ribosome entry site.

Transcription of HVTK (TK) and particular oncogenes, e.g., BCR-ABL p210 was confirmed by quantitative Real-Time PCR as described under Materials and methods. In Sca1+Lin− cells (i.e. cells expressing Sca1 but not Lin), expression of HVTK (TK) and BCR-ABL p210 was significantly higher than in Sca1−Lin+ cells (FIG. 1, FIG. 29). Interestingly, this was found to be the case both before and after disease onset, indicating that the oncogenes themselves are not involved in cancer maintenance (cf. also Example 14). Equivalent observations were made, e.g., in the Sca1-BCL6-IRES-HVTK mice with regard to HVTK and BCL6 transcription.

### Example 4

### SCA1/BCR-ABL p210 CSC Mice Represent the First Mouse Model to Reproduce the Pathology of Human CML and Blast Crisis

In one of the CSC mouse models of Example 3, a BCR-ABL p210 gene was inserted into the 5′-UTR of the SCA1 gene. These mice are also referred to as SCA1/BCR-ABL p210 mice.

It was found that these mice developed cancer that truly reproduced the symptoms and pathology of human CML, with respect to the chronic (myeloproliferative) phase, the blast crisis, the size of HSC and the response to treatment with Gleevec™.

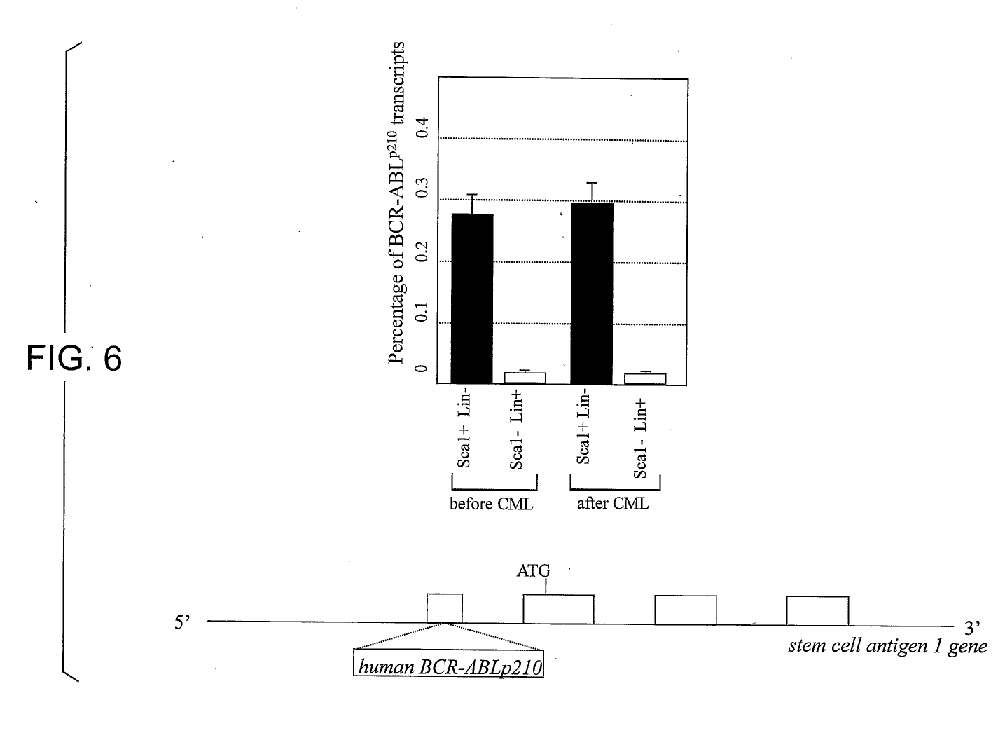

In these SCA1/BCR-ABL p210 mice, the percentage of BCR-ABL p210 transcripts was found to be high (0.20-0.35%) in Sca1+Lin− cells and low (<0.1%) in Sca1−Lin+ cells, both before and after CML (FIG. 6).

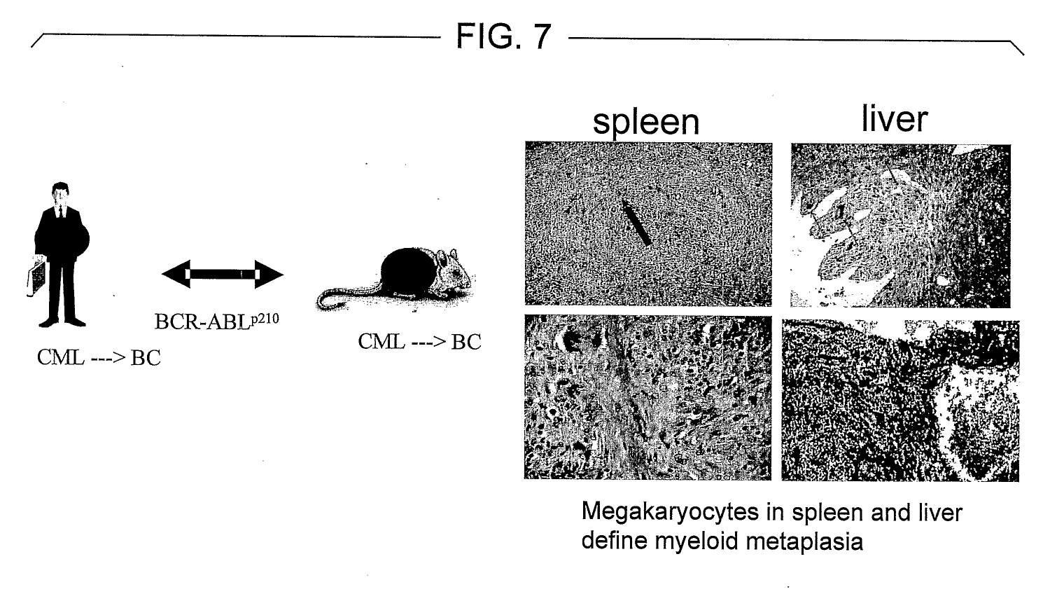

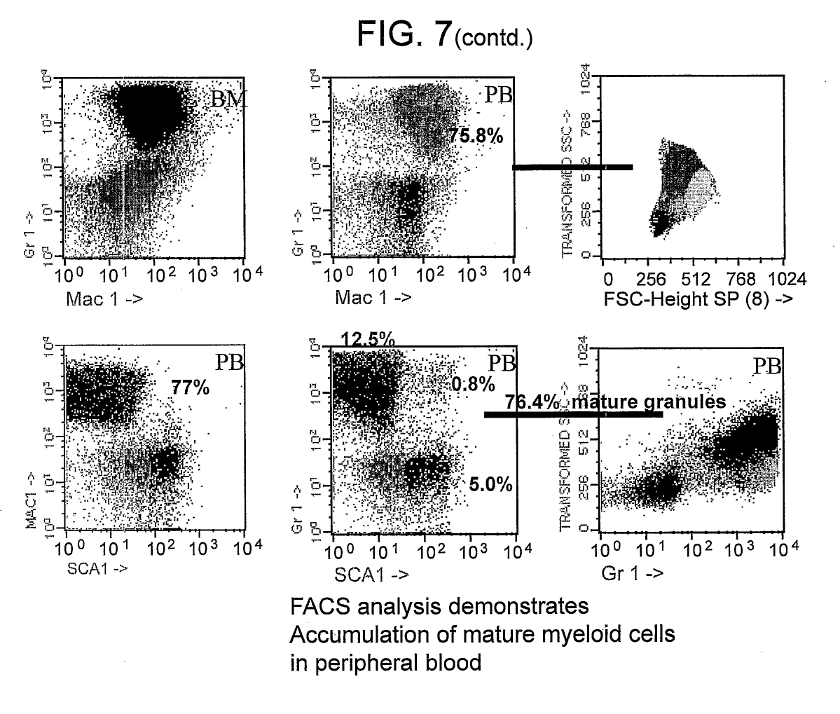

A histological and histochemical analysis of the spleen and liver from these mice revealed megakaryocytes that were characteristic of myeloid metaplasia (FIG. 7).

Analysis of peripheral blood samples from these mice by flow cytometry (Flow activated cell sorting, FACS) revealed an accumulation of mature myeloid cells (FIG. 7).

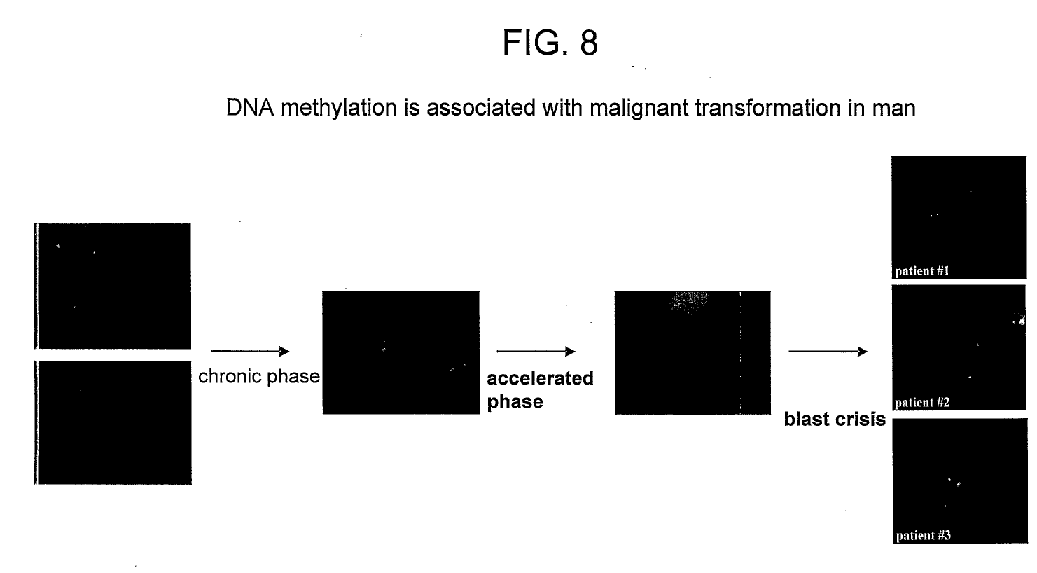

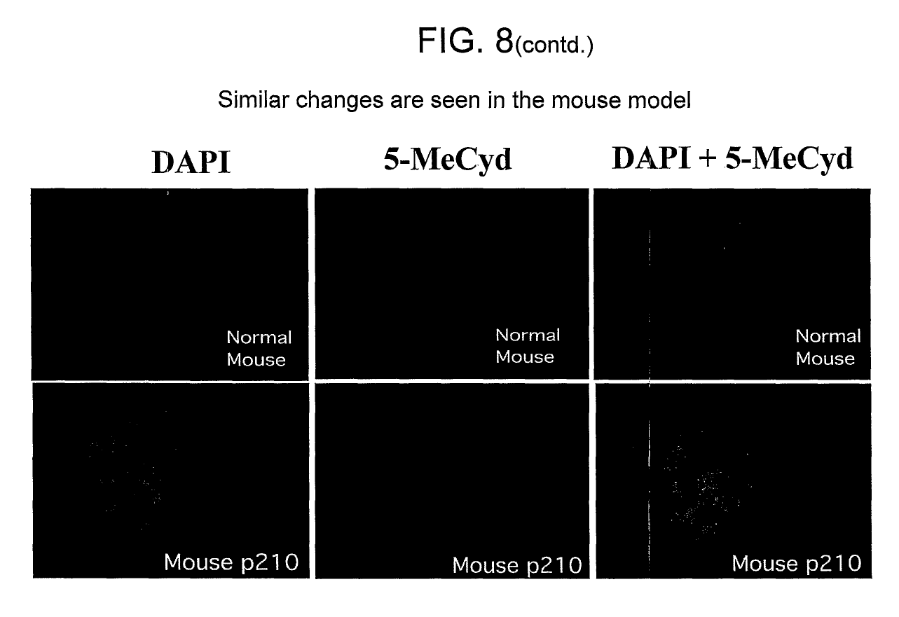

Blast cells from the CSC mouse model were analysed for DNA methylation (5-methyl cytidine, 5-MeCyd). Comparison of DAPI-stained cells with 5-MeCyd detection showed that, as in human CML, DNA methylation as is characteristic of malignant transformation occurs in the CSC mouse model (FIG. 8).

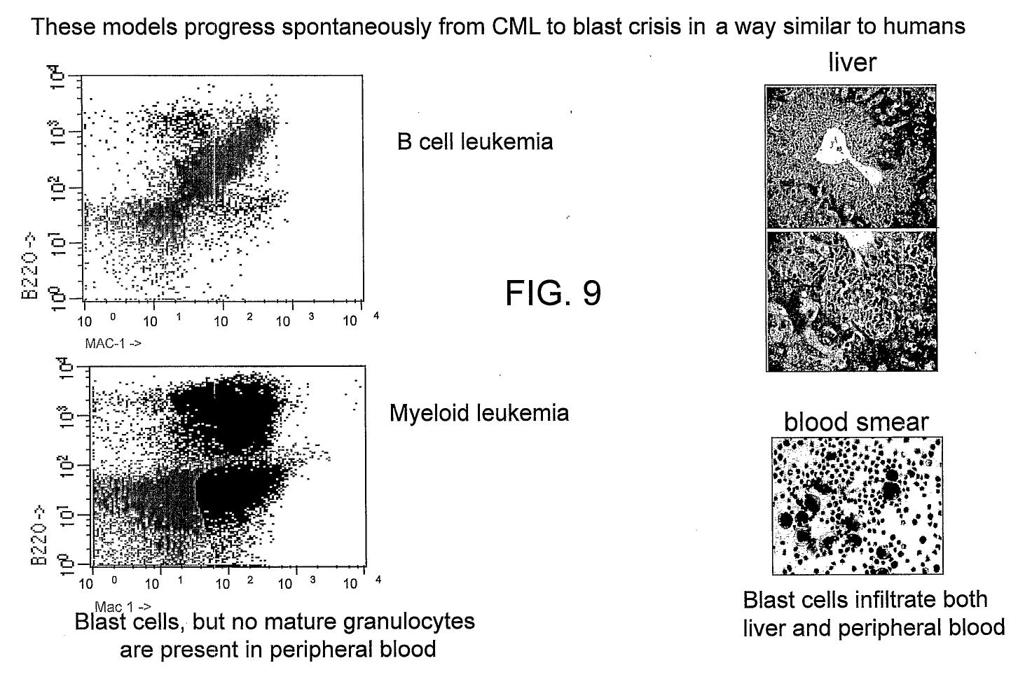

It was found that the SCA1/BCR-ABL p210 mice progress spontaneously from CML to blast crisis, in a manner that resembles progression of the disease in humans. Flow cytometric analysis demonstrated the simultaneous presence of blast cells and absence of granulocytes in these CSC model mice. The results of such an analysis are shown for B-cell leukaemia and myeloid leukaemia in FIG. 9. Moreover, histochemical analysis revealed the infiltration of liver and peripheral blood by blast cells (FIG. 9).

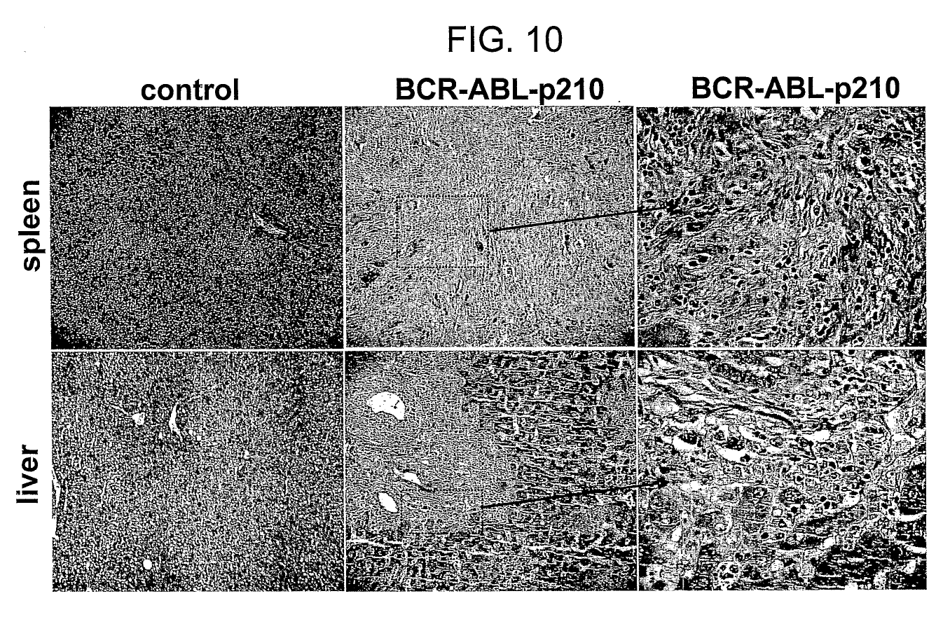

Liver and spleen samples were stained using Masson's trichrome protocol, revealing in SCA1/BCR-ABL p210 mice the fibrosis of the liver and spleen that is also observed in human CML/blast crisis patients (FIG. 10).

### Example 5

### SCA1/BCR-ABL p190 CSC Mice Reproduce the Pathology of Human B-ALL

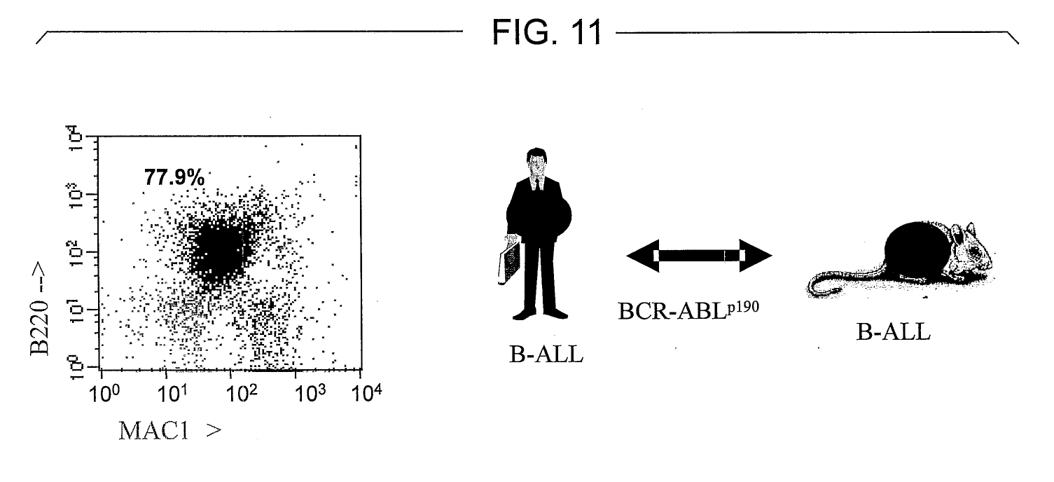

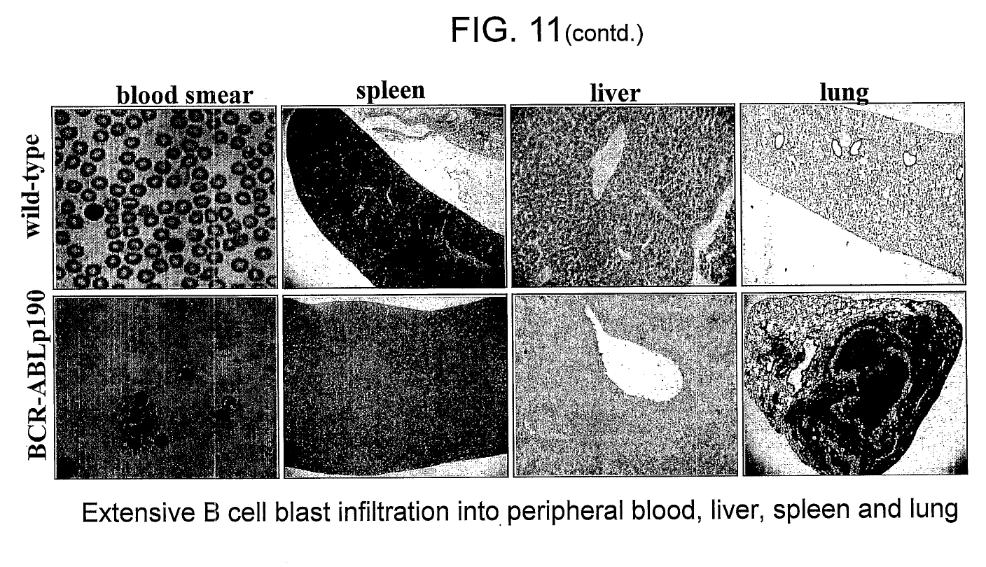

When ACR-ABL p190 was used as the oncogene in the CSC mouse model, the mice developed a pathology corresponding to B-ALL in humans. Extensive B cell blast infiltration into the peripheral blood, the liver, spleen and lungs of the mice was observed by flow cytometric and histochemical analysis (FIG. 11).

### Example 6

### SCA1/LMO2-RHOM2 CSC Mice Reproduce the Pathology of Human T-ALL

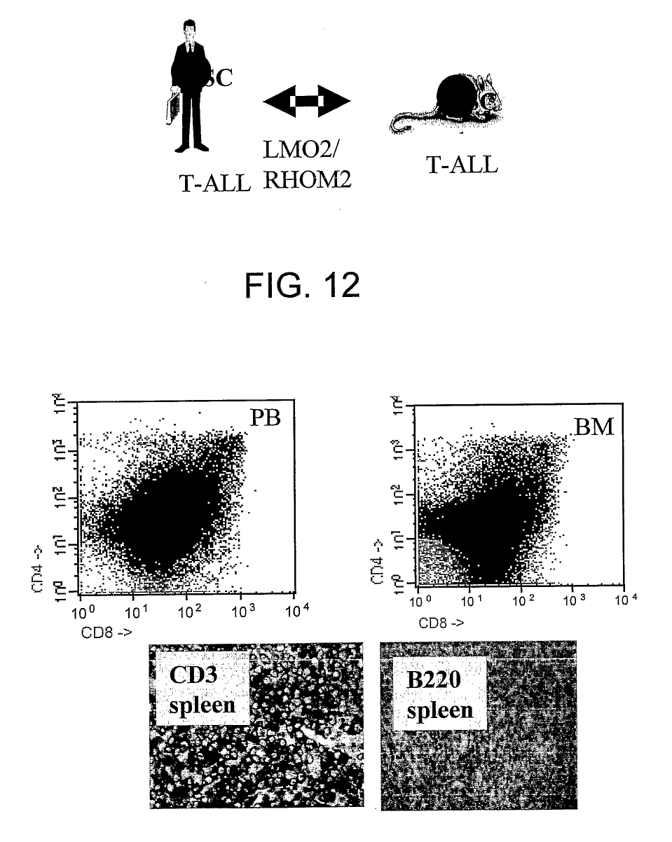

When LMO2-RHOM2 was used as the oncogene in the CSC mouse model, the mice developed a pathology corresponding to human T-ALL. The development of T-cell leukaemia and blast dissemination into tissues and peripheral blood was identical to the human pathology. In FIG. 12, the results of histochemical analysis of samples from thymoma, liver, kidney, testes and spleen tissue, as well as flow cytometric results from peripheral blood and bone marrow samples, are shown to demonstrate this typical development of T-ALL.

### Example 7

### SCA1/Maf-B CSC Mice Reproduce the Pathology of Human Multiple Myeloma

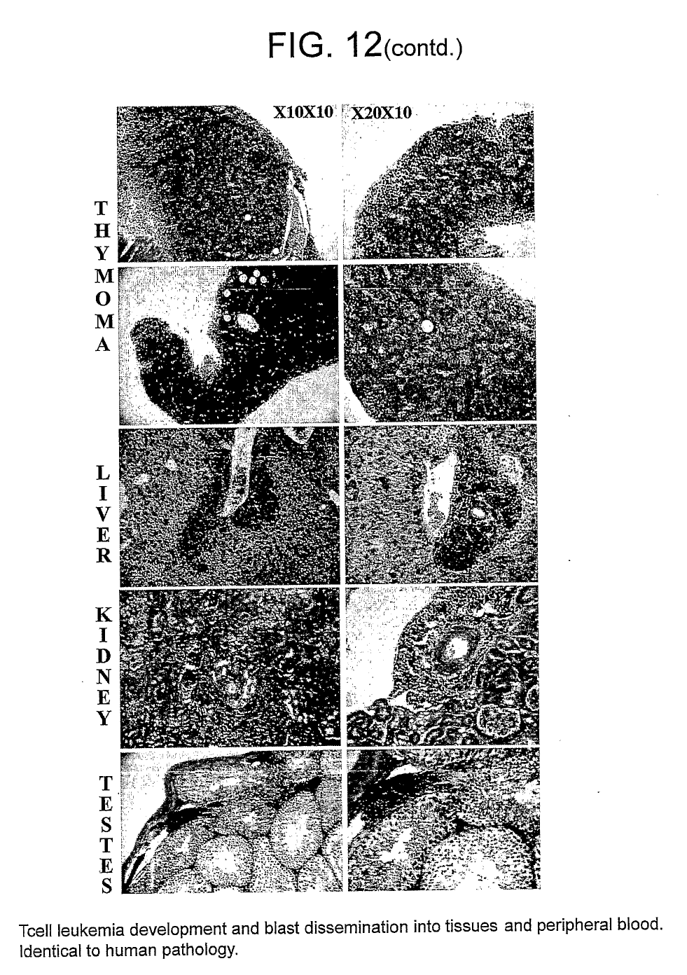

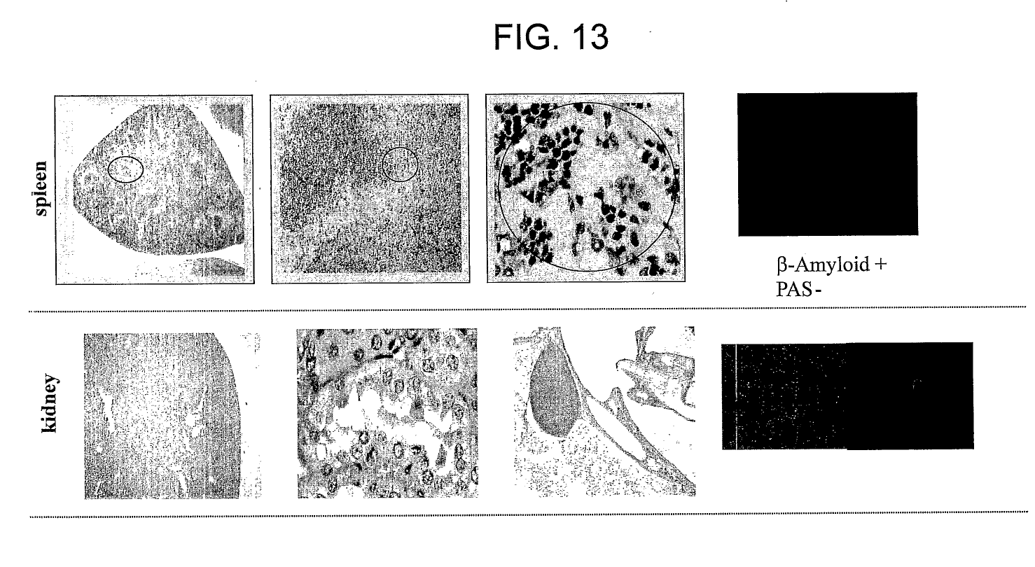

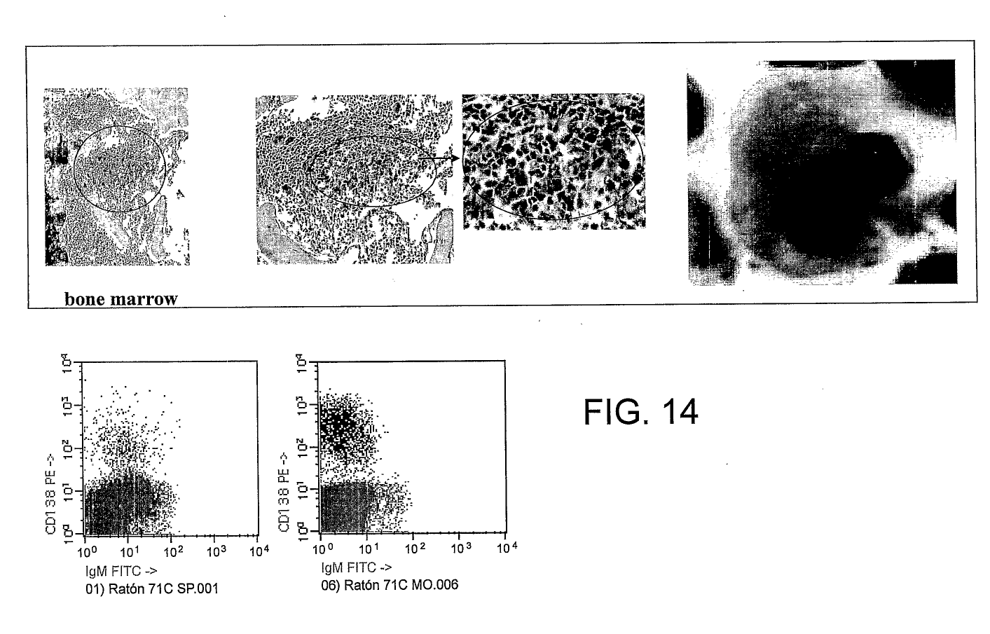

When Maf-B was used as the oncogene in the CSC mouse model, the mice developed a pathology corresponding to human multiple myeloma. Histochemical analysis revealed plasma cell infiltration into the spleen and kidney, with pathological indications of kidney failure (FIG. 13). Moreover, plasma cells were identified in bone marrow and peripheral blood (FIG. 14).

### Example 8

### SCA1/BCL6 CSC Mice Reproduce the Pathology of Human Lymphoma

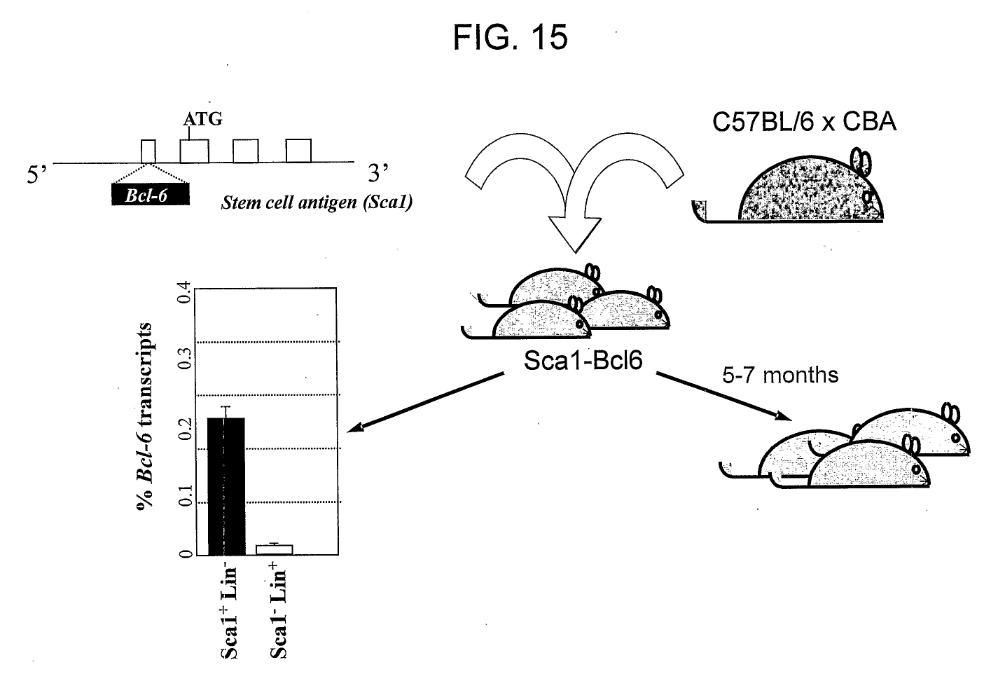

When BCL6 was used as the oncogene in the CSC mouse model (mouse models were obtained from C57BL×CBA mice), the mice developed a pathology corresponding to human lymphoma. SCA1/BCL6 mice developed lymphoma after about 5-7 months. The percentage of BCL6 transcripts was found to be high (0.2-0.3%) in Sca1+Lin− cells and low (<0.1%) in Sca1−Lin+ cells (FIG. 15).

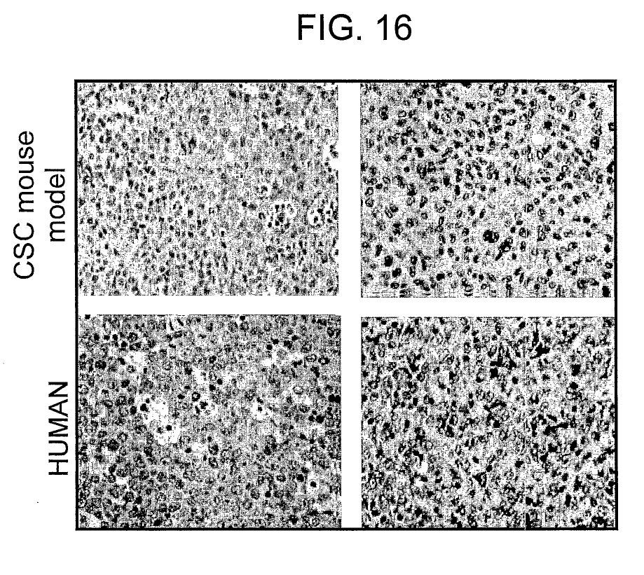

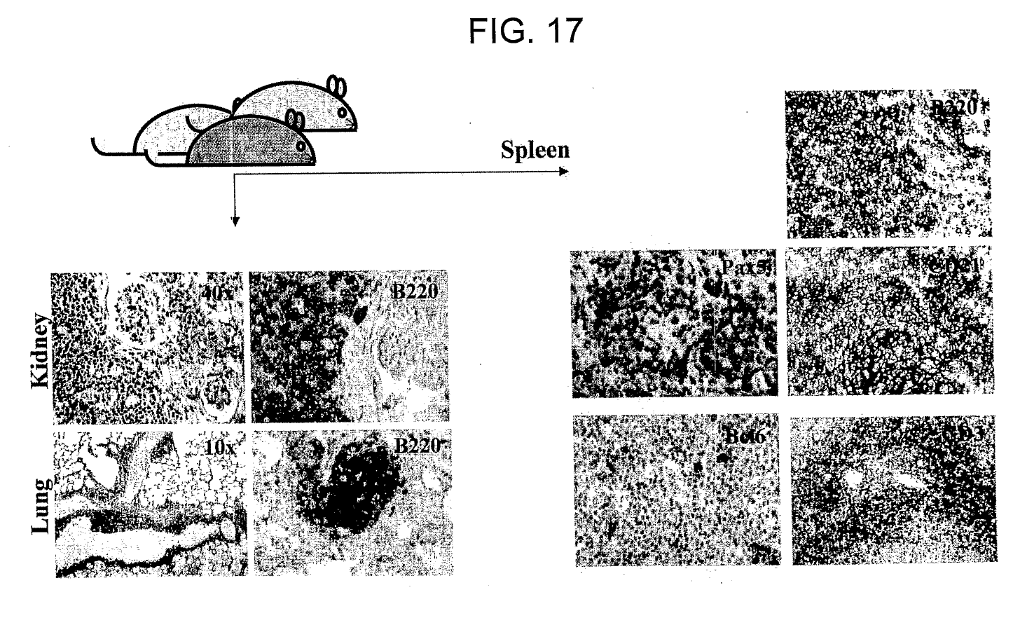

Histochemical analysis of spleen tissue samples demonstrated that the diffuse large B cell lymphoma in the CSC mouse model accurately mimics the human pathology (FIG. 16). Pax5+ and CD21+ B cell infiltration into lung and kidney tissue was also observed by histochemical staining (FIG. 17). B cells PAX5+, B220+, CD21+) did not express BCL6 in this CSC mouse model, as was shown by histochemical staining of spleen tissue samples (FIG. 17).

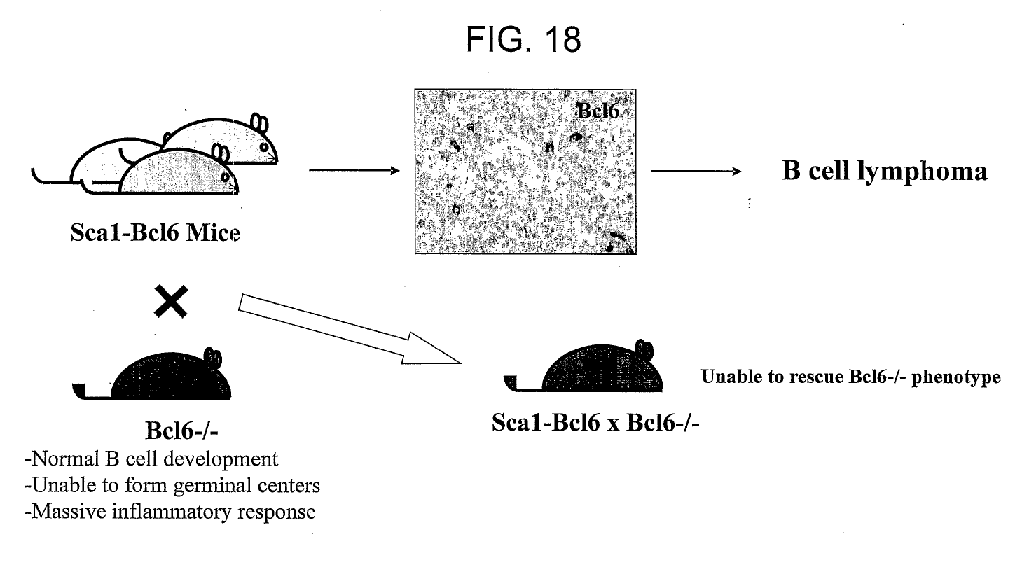

Crossing of SCA1/BCL6 (B cell lymphoma) mice with BCL6−/− mice did not lead to the rescue of the Bcl6−/− phenotype in the latter mice (FIG. 18). In the SCA1/BCL6×BCL6−/− progeny, development of B cells was normal. Moreover, the progeny mice were unable to form germinal centres and were characterised by a massive inflammatory response.

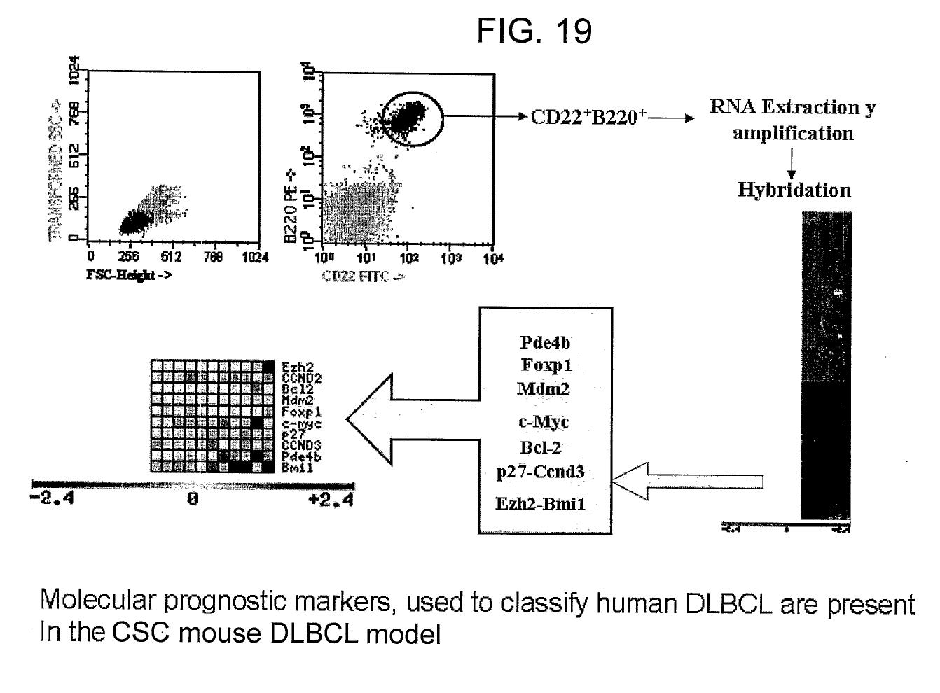

On the molecular level, the B cell lymphomas of the CSC model mice were also very similar to human B cell lymphomas. CD22+B220+ cells were isolated by flow cytometry. RNA extraction and amplification, as well as hybridisation was carried out as described in Materials and methods. As is shown in FIG. 19, molecular prognostic markers that are commonly used to classify human DLBCL, such as Pde4b, Foxp1, Mdm2, c-Myc, Bcl2, p27-Ccnd3 or Ezh2-Bmi1, are also present in the SCA1/BCL6 CSC mouse model of DLBCL.

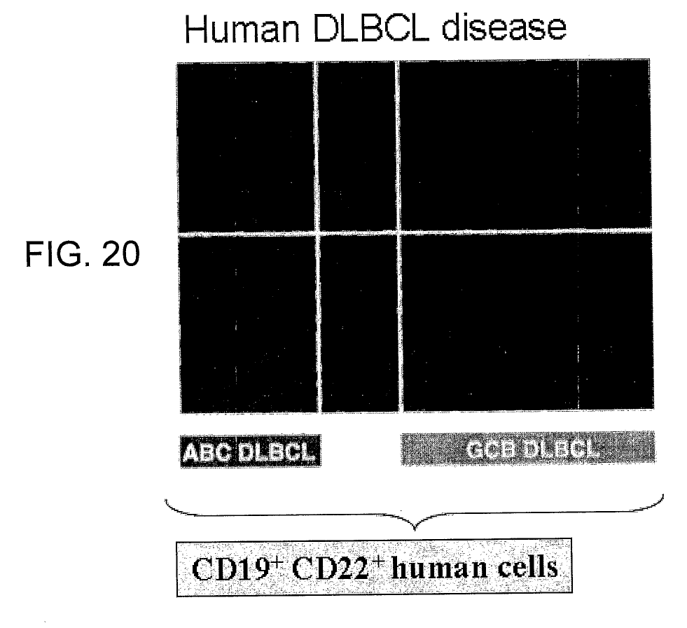

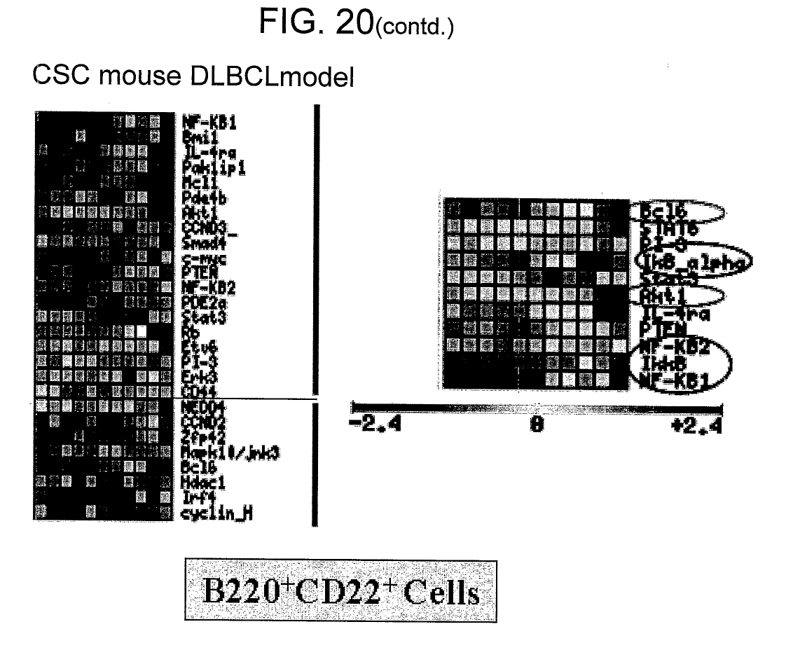

In particular, however, it was found that DLBCL cells (B220+CD22+) of the SCA1/BCL6 CSC mouse model do not express BCL6. In this respect, said CSC mouse model resembles human “activated B cell-like” (ABC) CD19+CD22+ DLBCL cells, rather than “Germinal centre B cell-like” (GCB) CD19+CD22+ DLBCL cells (FIG. 20).

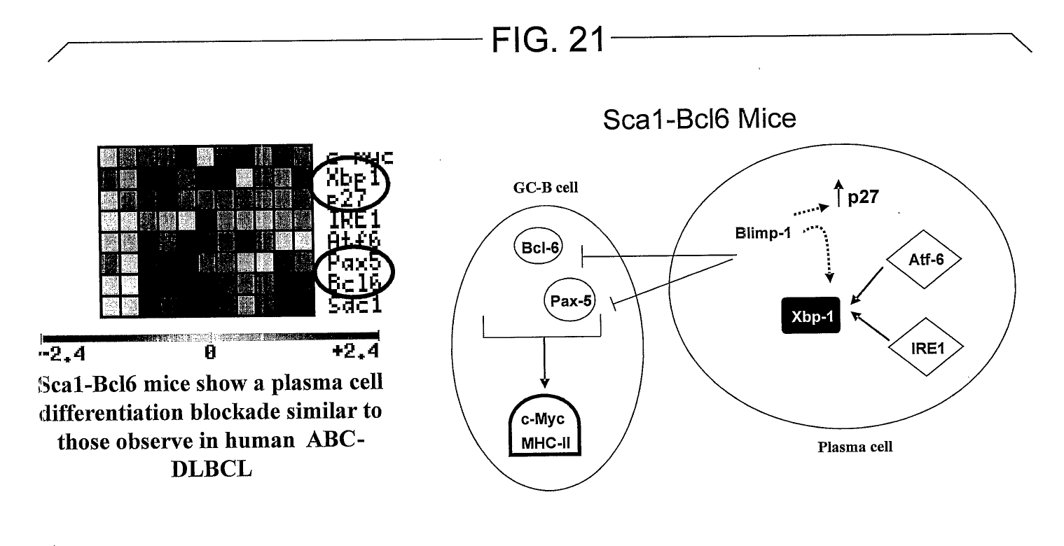

It was furthermore found that, similar to the pathology of human ABC DLBCL, SCA1/BCL6 mice exhibited a plasma cell differentiation blockade. RNA hybridisation assays as described under Materials and methods on germinal centre B cells and plasma cells from SCA1/BCL6 mice revealed that Xbp1 and p27 were up-regulated, whereas Pax5 and Bcl6 were down-regulated (FIG. 21).

### Example 9

### Oncogenic Activation in Solid Tissue Sca1+ Cells Leads to Solid Tissue Carcinomas in Mice

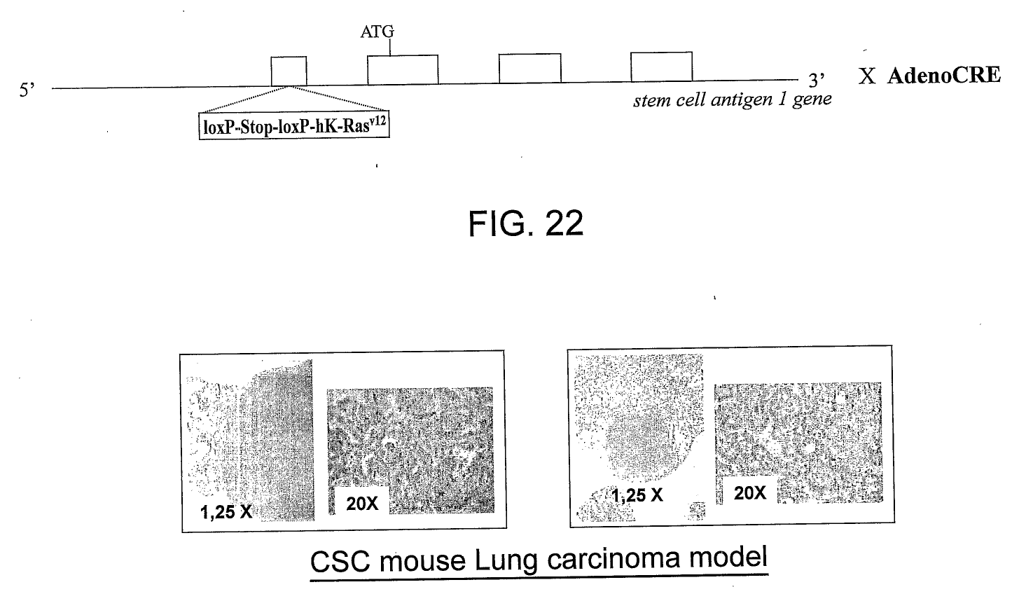

A loxP-Stop-loxP-hK-Ras^{v12} gene construct was inserted into the 5′-UTR of the SCA1 gene of a transgenic mouse, as shown in FIG. 22. The oncogene could thus be activated by infection with Adeno-Cre. This CSC mouse model developed lung carcinomas of the non-small cell lung cancer (NSCLC) type, which were observed 3-5 months after Adeno-Cre infection.

### Example 10

### Haematopoietic CSC Mouse Models Also Develop Solid Tumours

The inspection of the various CSC mouse models of haematopoietic cancers revealed that these mice also developed solid tumours.

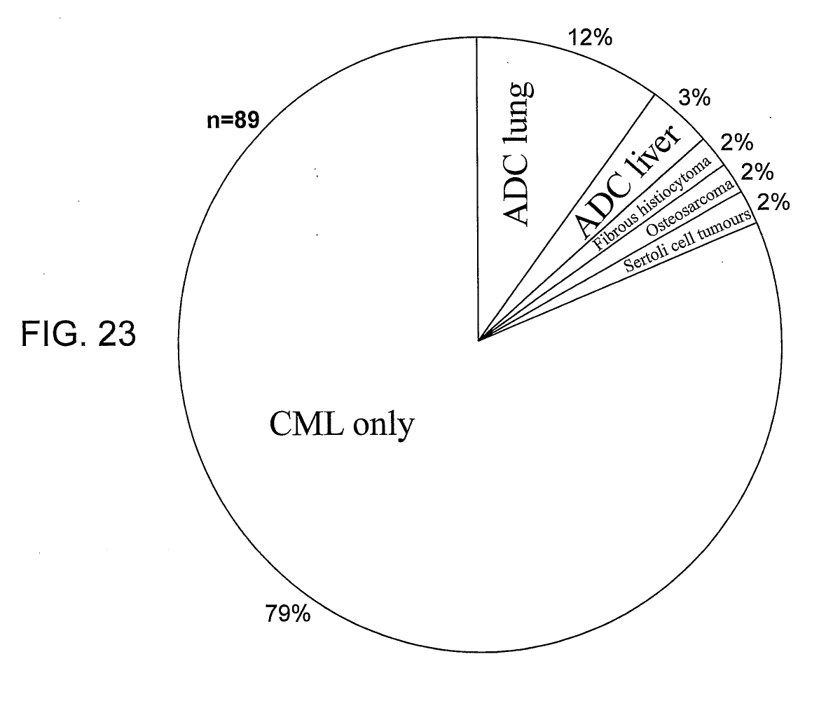

In the CSC mouse model of CML (cf. Example 3 and Example 4), of 89 mice in total, 79% developed CML only. However, lung adenocarcinomas (ADC) were observed in 12%, liver ADC in 3%, fibrous histiocytomas in 2%, osteosarcomas in 2% and Sertoli cell tumours in 2% of the total sample of these mice (FIG. 23).

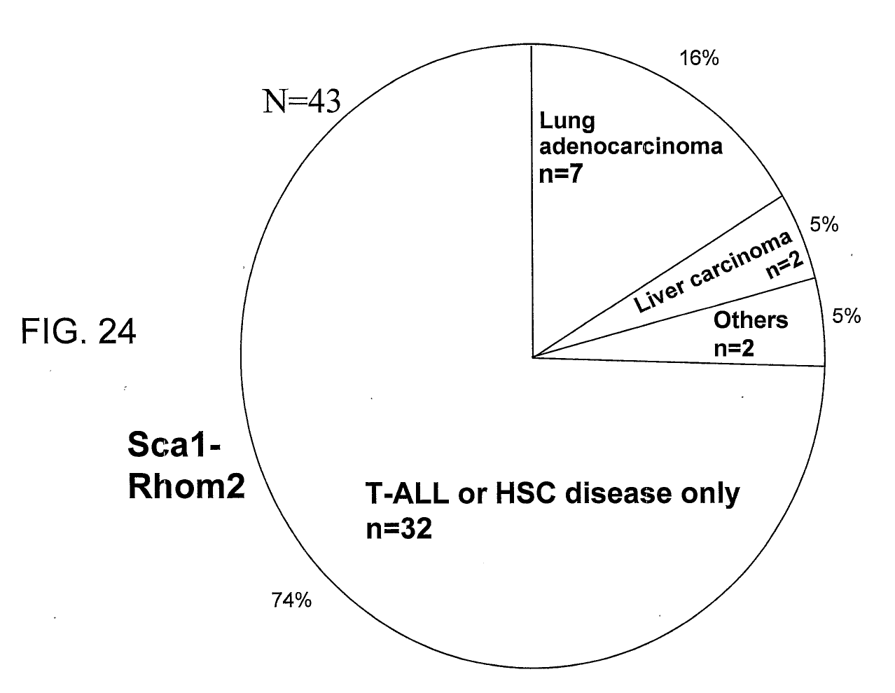

In the SCA1/RHOM2 T CSC mouse model of T-ALL (cf. Example 3 and Example 6), of 43 mice in total, 74% (32 mice) developed T-ALL only. However, lung ADC were observed in 16% (7 mice) and liver carcinomas in 5% (2 mice) of the total SCA1/RHOM2 sample. Other cancers were observed in 5% (2 mice; see FIG. 24).

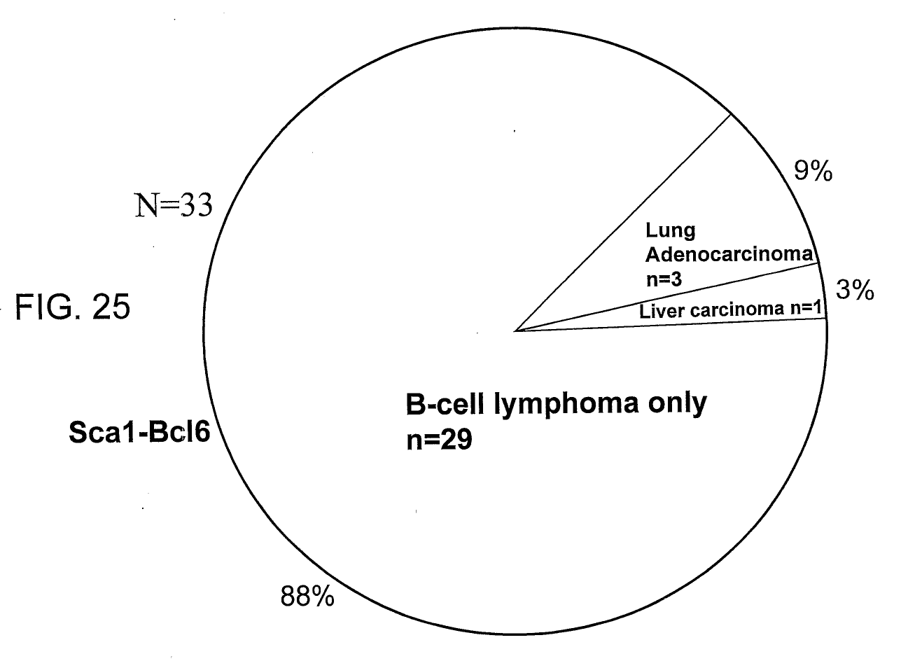

In the B-cell lymphoma model (cf. Example 8), of a sample of 33 mice in total, 88% (29 mice) developed B-cell lymphoma only. However, lung ADC were observed in 9% (3 mice), and liver carcinomas were observed in 3% (1 mouse; see FIG. 25).

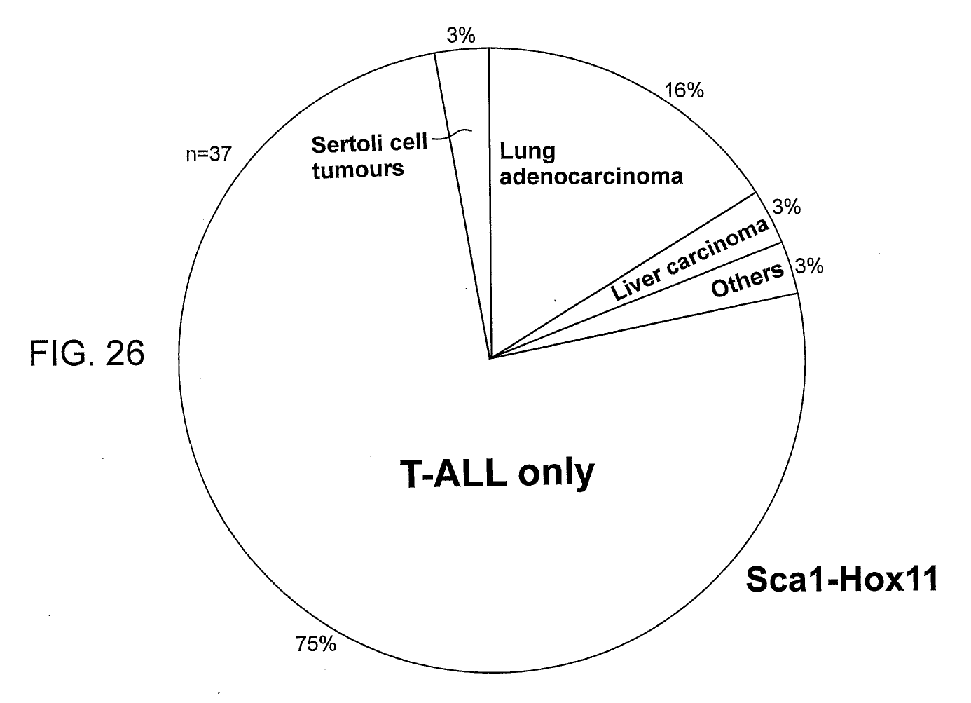

In the SCA1/Hox11 CSC mouse model of T-ALL (cf. Example 3), of a sample of 37 mice in total, 75% developed T-ALL only. However, lung ADC were observed in 16% of these cases. Liver carcinomas and Sertoli cell tumours were each observed in 3% of these mice. Other cancers were observed in further 3% of these mice (see FIG. 26)

### Example 11

### Modelling of Carcinoma Development by Reprogramming SCA1

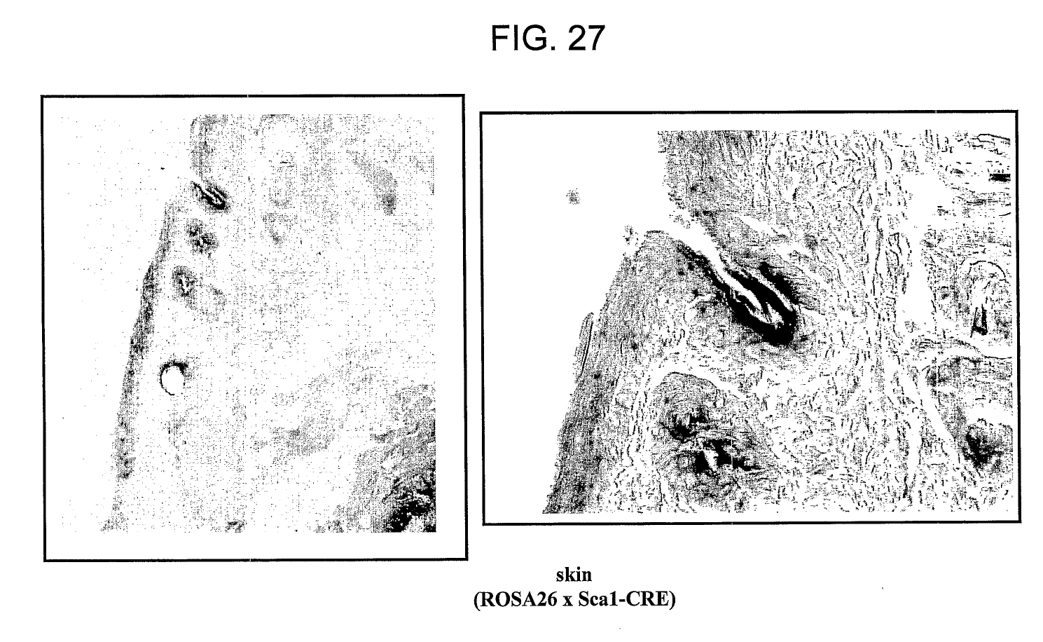

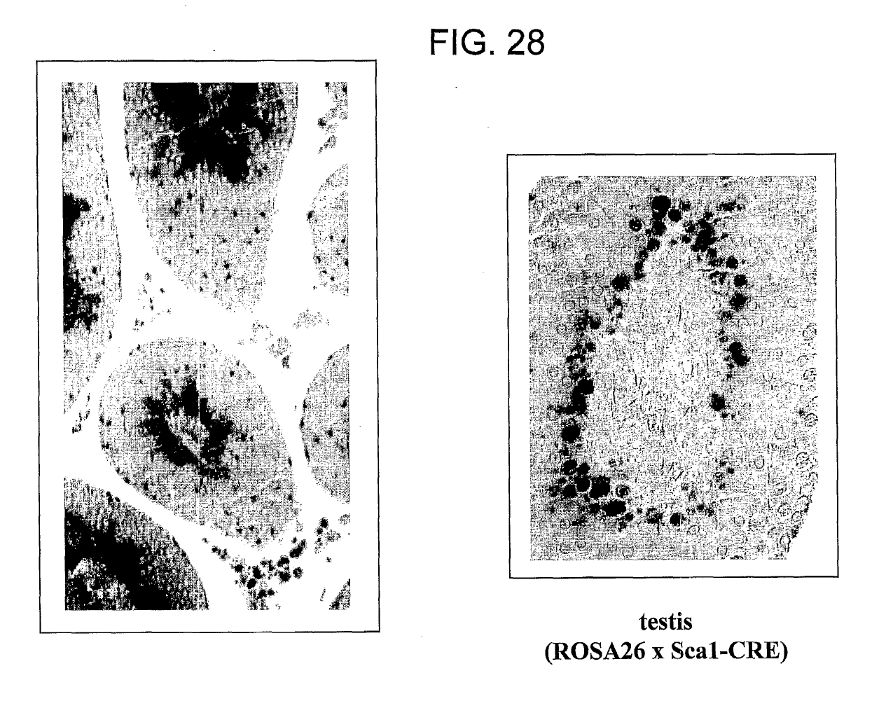

A mouse was generated, wherein Cre recombinase was inserted into the 5′-UTR of the Sca1 gene. These mice were crossed into the ROSA26 reporter strain, in which Bga1 activity/expression is prevented by a stop cassette “floxed” by loxP sites. Sections of skin and testis of the Rosa26×Sca1-CRE mice thus generated were subjected to B-gal staining. Regions stained blue were those regions in which stem cells are normally located (FIG. 27 and FIG. 28).

### Example 12

### Proof that CSC Ablation is an Effective Treatment Strategy for Cancer

CSC mouse cancer models (Sca1-HVTK-IRES-BCR-ABLp210 and Sca1-BCL6-IRES-HVTK mice) were used to address the question of whether the selective ablation of Sca1-positive cells is effective in the therapy of cancer.

Mouse models involving transgenic constructs including HVTK were generated on the basis of the following rationale: Expression of HVTK induces conversion of the prodrug form of the nucleoside analogue ganciclovir into the active, phosphorylated form of the drug. Phosphorylated ganciclovir is incorporated into the DNA of replicating cells causing irreversible arrest of said cells at the G2/M checkpoint followed by apoptosis, as described in Rubsam L. Z. et al., (1998) Cancer Res 58 3873-3882.

As HVTK is, in this CSC mouse cancer model, expressed specifically in CSC, it is possible to ablate CSC specifically in these mice by ganciclovir treatment.

This allows the testing of the hypothesis that CSC are the source of the occurrence and recurrence of cancer, as this hypothesis would predict that cancer can be treated efficaciously and with lasting effect by selectively killing CSCs.

The mice were treated either with ganciclovir (GCV) or with placebo, as described under materials and methods. For each mouse model, 60 mice were injected daily with ganciclovir, and 30 mice were used as a control and injected daily with saline (placebo).

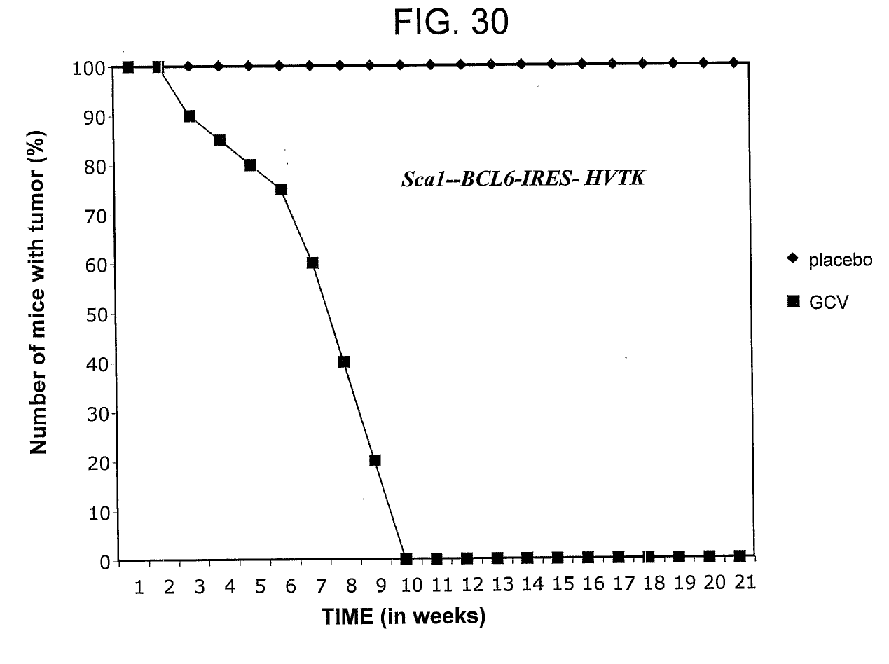

In the Sca1-BCL6-IRES-HVTK mice, treatment with ganciclovir led to the elimination of all tumours after 10 weeks (FIG. 30), whereas the number of mice with tumours remained unchanged (100%) upon treatment with placebo. Mice from which tumours had disappeared were monitored for an additional 11 weeks during which no tumour recurrence was observed.

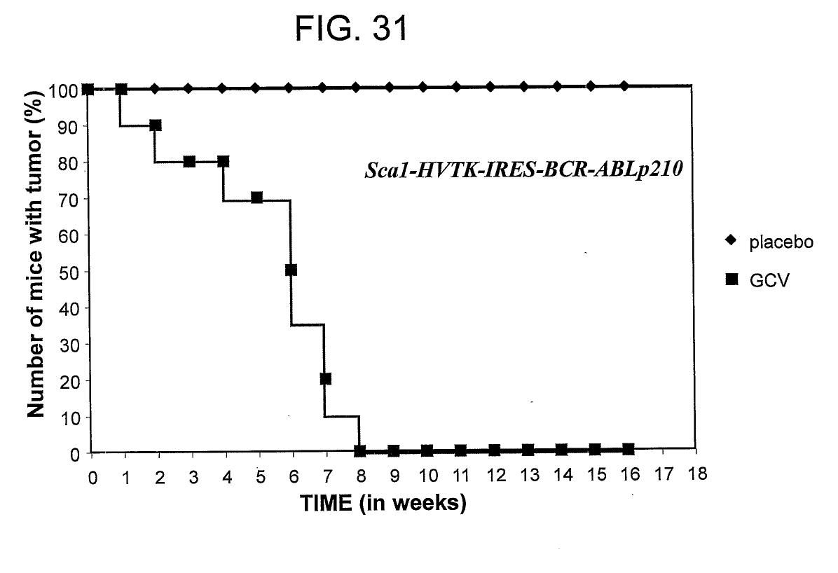

In the Sca1-IRES-HVTK-BCR-ABL p210 mice, equivalent results were obtained (FIG. 31). In the 60 Sca1-IRES-HVTK-BCR-ABL p210 mice treated with ganciclovir, tumours disappeared completely after 8 weeks of treatment, and did not recur during 8 weeks of further monitoring. In the 30 mice treated with a saline placebo as controls, no reduction in the number of tumours was observed.

This experiment confirmed the CSC hypothesis and demonstrated for the first time that killing CSC is an effective therapeutic strategy for cancer treatment, with broad potential applications.

### Example 13

### Proof that Cancer is Maintained by CSC

In order to identify the cell population responsible for cancer maintenance, Sca1+Lin− cells were purified from the bone marrow (these cells correspond to undifferentiated cells, CSC), and B220+CD22+ cells were purified from the spleen of SCA1/BCL6 mice (these cells correspond to differentiated tumour cells).

These cells were transplanted by tail vein injection into syngenic healthy mice. For each of the SCA1/BCL6 phenotype and the control, six syngenic healthy animals were transplanted with 10,000 Sca1+Lin− cells, and six were transplanted with 1,000 Sca1+Lin− cells. For B220+CD22+ cells, the same numbers of test animals were used, but 10⁵ and 10⁶ cells were transplanted.

Mice reconstituted with B220+CD22+ cells did not develop lymphomas. However, mice reconstituted with Sca1+Lin− cells developed B-cell lymphomas. The disease developed 74(±11) days on average after transplantation in the six mice that received 10,000 cells, and 81 (±9) days on average after transplantation in the six mice that received 1,000 cells. No lymphomas were detected in any of the control mice.

These results showed that the disease is maintained and propagated by Sca1⁺Lin− cells (CSCs).

### Example 14

### Targeting BCR-ABL does not Kill Cancer Stem Cells

The inventors investigated whether BCR-ABL p210 is required for tumour malignancy.

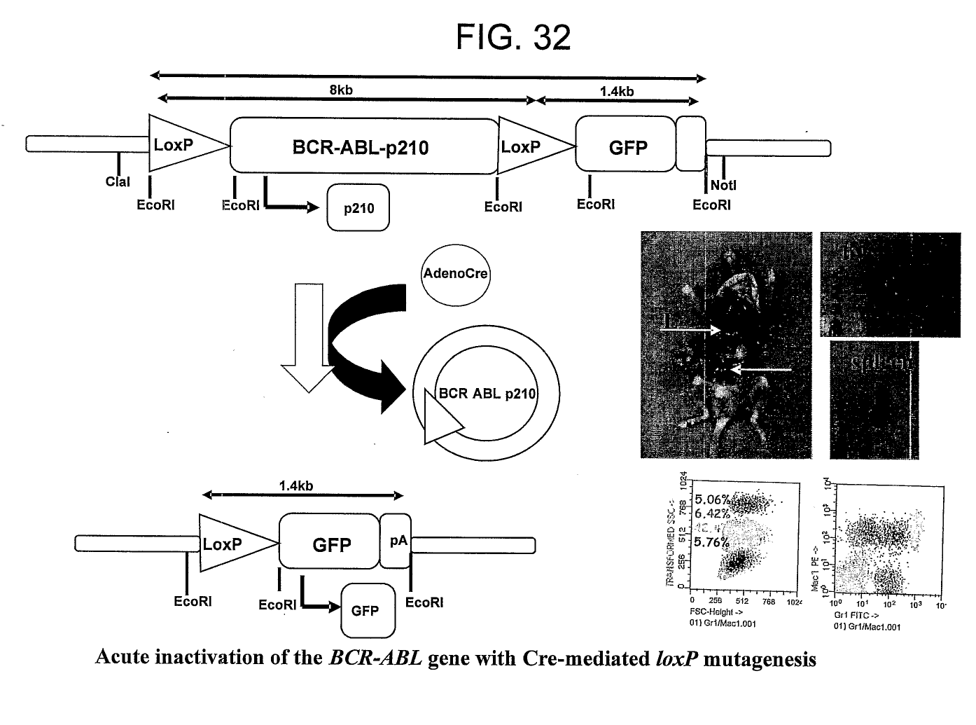

To this end, a mouse strain was constructed in which, in the 5′-UTR of the SCA1 gene, the BCR-ABL p210 gene was placed between LoxP sites and followed by a gene for GFP (green fluorescent protein). The BRC-ABL p210 gene could thus be inactivated by Cre-mediated LoxP mutagenesis. The location of the GFP gene downstream of the second (downstream) LoxP site was such that GFP would be expressed only upon excision of the BCR-ABL p210 gene by the action of Cre recombinase upon the LoxP loci (see FIG. 32).

Bone marrow (BM) cells were isolated from 8-10 week-old male donor CSC model mice carrying the BCR-ABL construct described above and schematically represented in FIG. 32. The isolated bone marrow cells were propagated in cell culture and infected with adenovirus expressing the Cre recombinase (Adeno-Cre). GFP was also purified from these cells.

Cultured and infected bone marrow cells expressing green fluorescent protein (GFP+cells) were isolated by flow cytometry (see under Materials and methods) and transplanted into syngenic, healthy female wild-type mice that had previously been irradiated at a dose sufficient to kill endogenous bone marrow cells (˜960 rads).

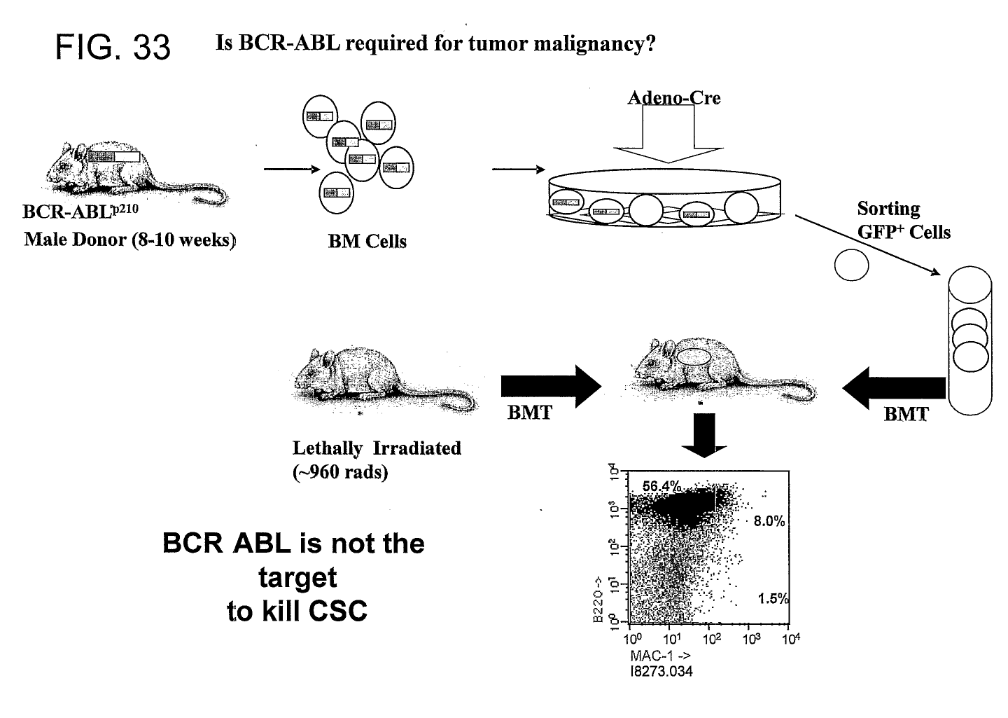

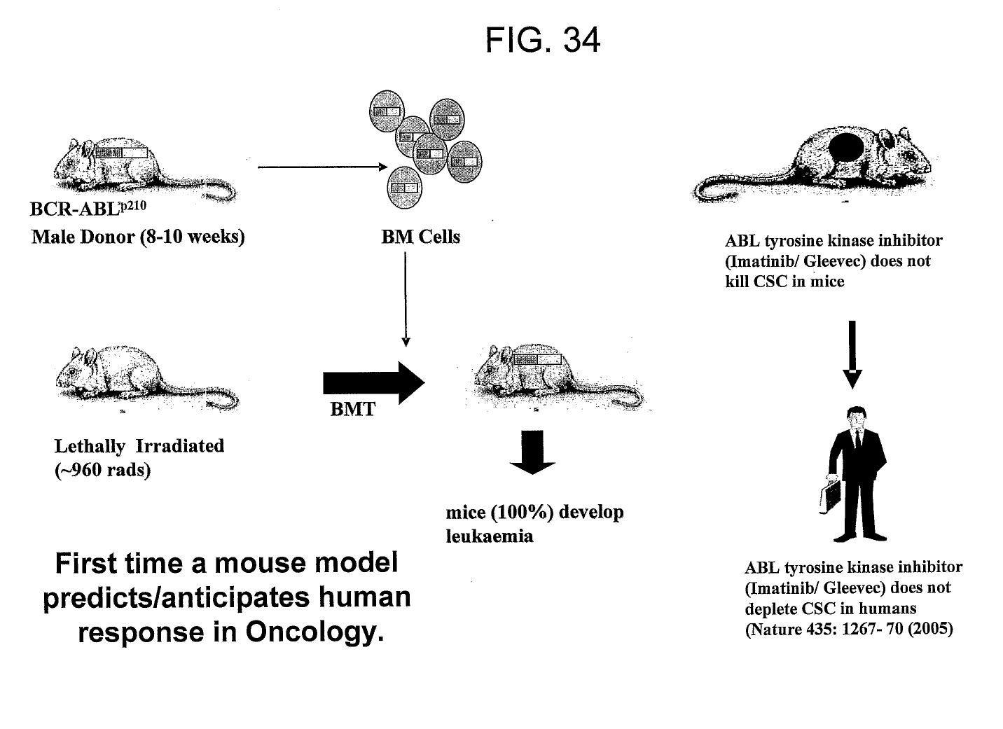

The transplanted bone marrow cells (GFP-expressing cells) from which the BRC-ABL p210 gene had been eliminated by the genetic Adeno-Cre approach correspond to cells in which Gleevec™ has acted with 100% efficacy. Nonetheless, the recipient mice developed leukemias, as is demonstrated by the flow cytometry (FACS) diagram of FIG. 33).

It was concluded that BCR-ABL p210 is not a suitable target for the treatment of cancer. This can be explained by the fact that the removal of that gene does not eliminate CSC.

### Example 15

### Stem Cell Driven Cancers in the Mice Models and in Patients Respond in a Similar Manner to Therapy with Gleevec™

Bone marrow cells from BCR-ABL p210 mice were transplanted into lethally irradiated mice. All recipient mice developed chronic myeloid leukaemia (CML).

In a second study, bone marrow cells from BCR-ABL p190 mice were transplanted into lethally irradiated mice. In this case, recipient mice developed B-cell acute leukemia (B-ALL)

Disease was monitored by the presence of blast cells co-expressing both myeloid and B-cell markers. BCR-ABL p210 and BCR-ABL p190 expression was monitored by real time PCR as described under Materials and methods.

Gleevec™ (Imantinib, also termed STI571), is an ABL tyrosine kinase inhibitor. It has been suggested that, in humans, while Gleevec™ depletes differentiated cancer cells and may thus provide initial symptomatic relief, Gleevec™ does not deplete leukaemic stem cells (Michor F et al. Nature (2005) 435, 1267-70.). In line with the CSC hypothesis, patients treated with Gleevec™ thus typically relapse.

Both the BCR-ABL p210 bone-marrow recipient mice with CML and the BCR-ABL p190 bone-marrow recipient mice with B-ALL were treated with Gleevec™. Stock solutions of Gleevec™ were prepared freshly in water, sterile filtered and administered to mice by gavage twice a day as described under Materials and methods. Treatment of the mice with Gleevec™ or with placebo commenced on the day after leukaemia was confirmed (day 0). The Gleevec™ was administered by means of straight or curved animal feeding needles. Mice tolerated the therapy well and no interruption of therapy was necessary. Mice were clinically examined 3 times a week, and periodic peripheral blood counts were obtained by tail vein blood draw as indicated. For the survival analysis portion of this study, the death endpoint was determined either by spontaneous death of the animal or by elective killing of the animal because of signs of pain or suffering according to established criteria.

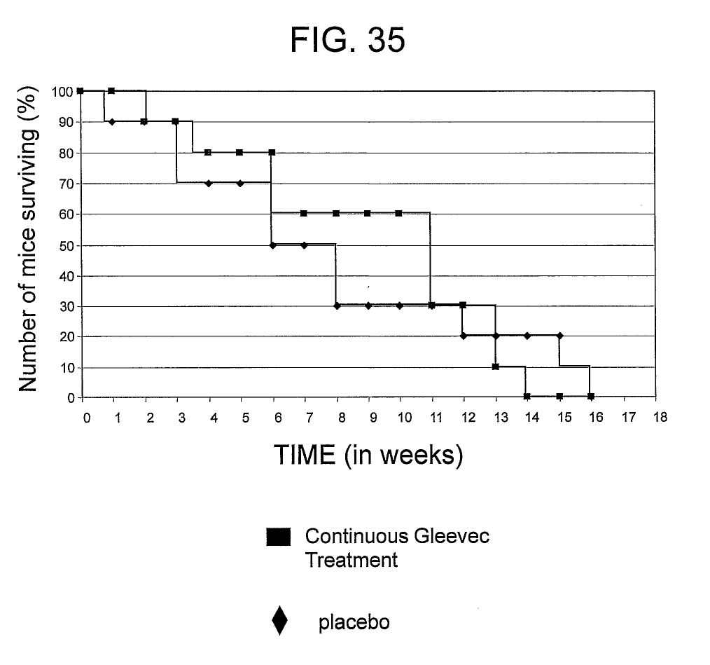

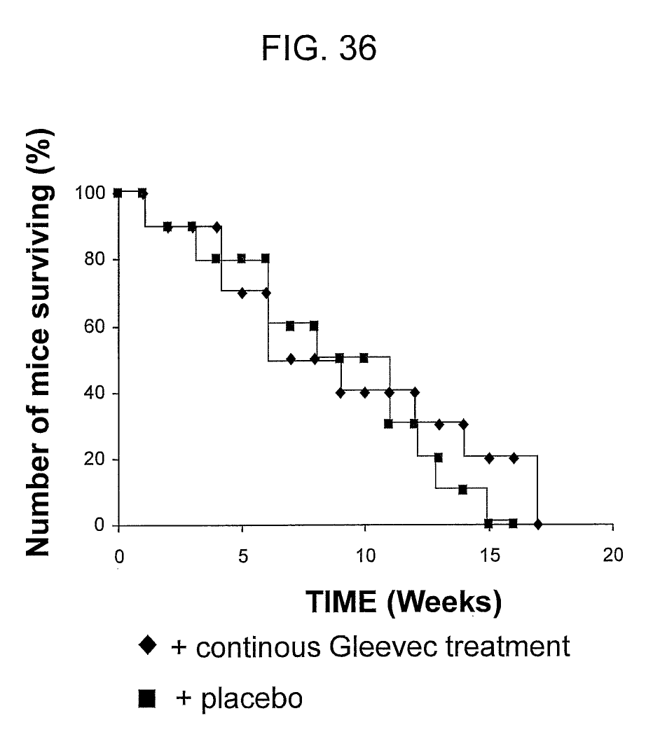

Treatment with Gleevec™ did not cure the leukaemia in the mice (see FIG. 35 for the BCR-ABL p210 CML model and FIG. 36 for the BCR-ABL p190 B-ALL model). In the CML mice, after 14 weeks, none of the mice treated with Gleevec™ survived. Of the mice treated with a placebo, 20% survived for 15 weeks (FIG. 35). In the B-ALL, none of the mice treated with placebo survived by week 15, and no mice treated with Gleevec™ survived by week 17.

In neither study were cancer stem cells found to be affected by the Gleevec™ treatment. Thus, the overall efficacy of the treatment of humans with Gleevec™ is mirrored in the CSC mouse models. This also suggested to the present inventors that relapse of human cancer patients after treatment with Gleevec™ is due to the same reasons for which Gleevec™ is ineffective in mice, i.e. that, unlike regular cancerous B cells, CSC are not affected by Gleevec™ treatment. This would be consistent with the CSC theory.

The inventors thus concluded that the present mouse models may be used to develop new generation drugs, corresponding to Gleevec™ but directed toward killing CSC.

### Example 16

### Stem Cell Driven Cancers in the Mice Models and in Patients Respond in a Similar Manner to Therapy with Doxorubicin

Doxorubicin is one of the most effective therapies in the treatment of human lymphomas. However, in particular in diffuse large B-cell lymphoma (DLBCL), doxorubicin therapy is often to be considered unsuccessful overall, as virtually all patients treated with doxorubicin relapse.

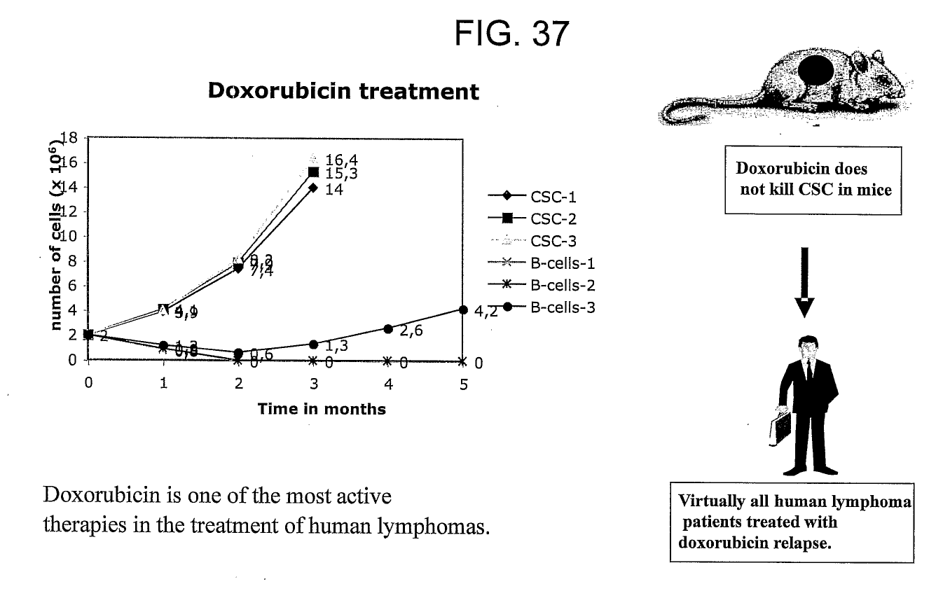

FIG. 37 shows the number of either CSC or B cells in cancer stem cell model mice (Sca1/BCL6) over a 3-5 month course of doxorubicin treatment. Whereas the number of B cells decreased or increased slightly, the number of CSC increased sharply by a factor of 7-8 over three months.

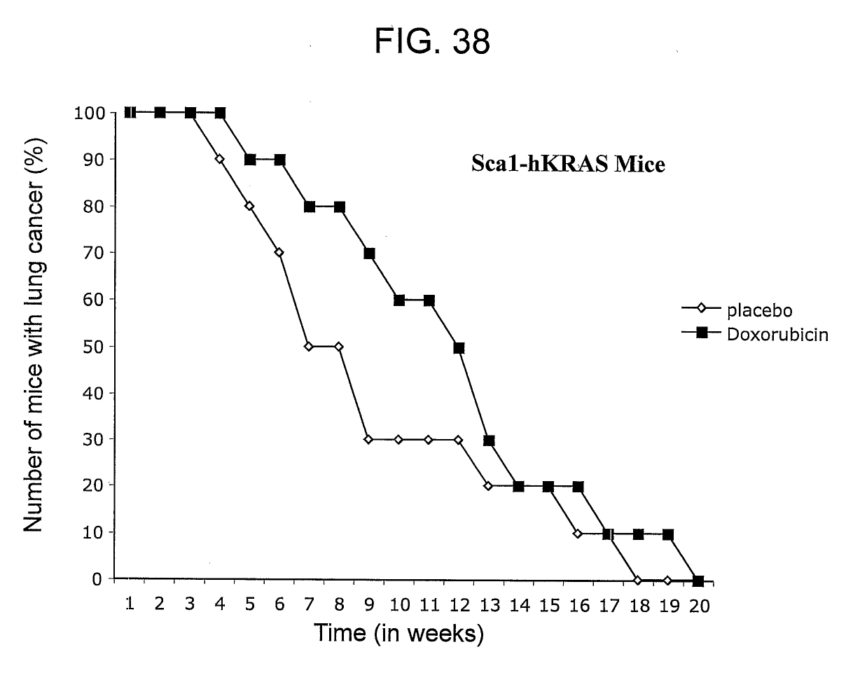

In FIG. 38, the survival of the CSC model lung cancer mice is plotted over a treatment period of 20 weeks. Of the mice treated with a placebo, none survived after 18 weeks, and of the mice treated with doxorubicin, none survived longer than 20 weeks.

Treatment with doxorubicin was therefore unsuccessful in the CSC mice. Consistent with the CSC theory, the inventors concluded that the most likely explanation was that doxorubicin did not kill the CSC in these mice.

Therefore, for doxorubicin as for Gleevec™, the inefficacy of treatment in humans is mirrored in the CSC mouse models.

### Example 17

### Stem Cell Driven Cancers in the Mice Models and in Patients Respond in a Similar Manner to Therapy with Melphalan Prednisolone

A melphalan prednisolone regimen is generally unsuccessful in the therapy of human multiple myeloma.

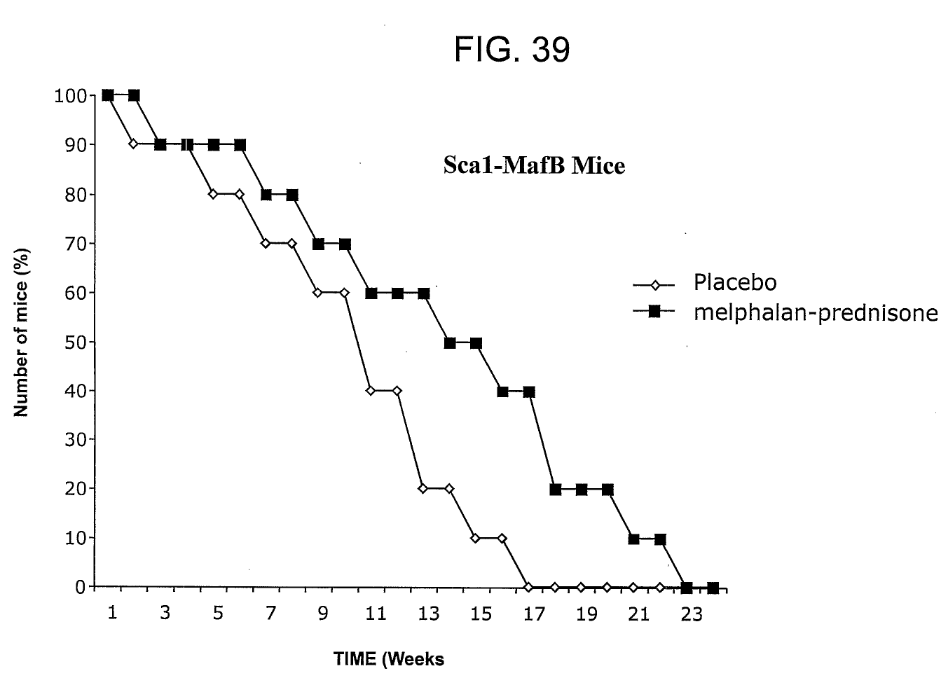

In FIG. 39, the survival of the CSC model lung cancer mice is plotted over a treatment period of 20 weeks. Of the mice treated with a placebo, none survived after 17 weeks, and of the mice treated with melphalan prednisolone, none survived longer than 22 weeks.

Treatment with melphalan prednisolone was therefore unsuccessful in the CSC mice. Consistent with the CSC theory, the inventors concluded that the most likely explanation was that melphalan prednisolone did not kill the CSC in these mice.

Therefore, for melphalan prednisolone as for doxorubicin and Gleevec™, human treatment efficacy is mirrored in the CSC mouse models.

### Example 18

### BEP Treatment is Successful in Both CSC Mouse Models and in Humans

BEP (Bleomycin, etoposide/VP16, cis-platin) cures human testicular carcinoma. Though this treatment is highly toxic, it is the only cancer treatment known to be capable of fully curing cancer.

In analogy to the mouse models of Example 3, a cancer stem cell mouse model was constructed wherein Sca1-positive cells (stem cells) were transformed into cancer stem cells by genomic insertion and expression of Snail, an oncogene.

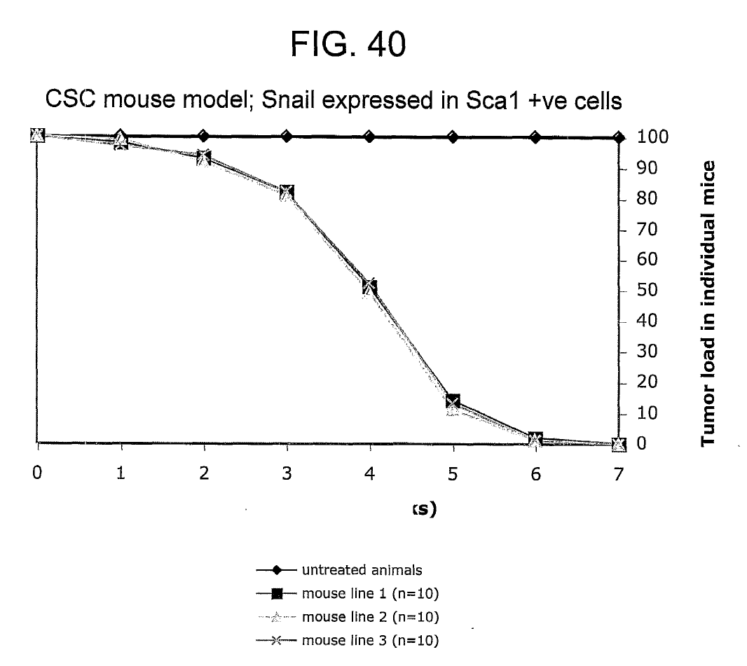

Treatment of the tumours these mice developed with BEP was highly successful: as shown in FIG. 40, in three different lines of mice (10 animals per line) the treatment reduced tumour load to zero after 7 weeks. In untreated model animals, the tumour load remained unchanged.

This experiment confirmed that the CSC mouse models mirror the response to cancer therapy in humans not only in the negative (as reported above, e.g., for treatment of CML and DLBCL with Gleevec™ and doxorubicin, respectively), but also in the positive cases.

### Example 19

### Differential Gene Expression Analysis in Cancer Stem Cells Versus Normal Stem Cells

Cells expressing ScaI were analysed and isolated by flow cytometry as described under Materials and Methods. From the stem cells thus isolated, RNA was extracted. Amplified antisense RNA (aRNA) was prepared from the extracted RNA, as indicated under Materials and methods, e.g, based upon the method of Van Gelder et al. (1990) Proc Natl Acad Sci USA 87: 1663-1667.

Hybridisation experiments with the RNA thereupon led to the following observations and conclusions.

CSC are significantly different from normal stem cells, with respect to the relative expression levels of genes such as Bmi1, Tel/Etv6, Tert, Gfi1, Notch1, β-catenin and Meis1. These genes, as well as Bcl6, p300, Stat5, Stat3 and Gata2, were, in general, upregulated in CSC.

CSC may be targeted and removed without the removal causing unacceptable side effects.

CSC form a homogenous cell population, inasmuch as, in CSC from different tumours, the same genes and/or groups of genes tend to be up- or down-regulated with respect to normal stem cells. Similarities in gene expression between different tumours led the inventors to conclude that therapeutic approaches that target and remove CSC can be used broadly, i.e. in a range of different cancers.

For example, the expression of Bcl6 in CSC in one mouse model leads to Diffuse Large B Cell Lymphoma (DLBCL). In another mouse model, the expression of BCR-ABL p210 CSC leads to Chronic Myeloid Leukaemia, However, these two mouse models display a rather similar gene expression profile, as similar genes are upregulated and downregulated in their CSC.

The following statements relate to additional aspects of the invention:
1. A murine stem cell comprising a gene created and/or activated by a genetic anomaly associated with a human pathology.
2. Stem cell according to aspect 1, wherein said stem cell is a cancer stem cell.
3. Stem cell according to aspect 1, wherein said human pathology is a human pathology of stem cell origin.
4. Stem cell according to aspect 1, wherein said human pathology is a human epithelial or mesenchymal cancer.
5. Stem cell according to aspect 4, wherein said human pathology is selected from lymphomas, leukaemias, sarcomas and carcinomas.
6. Stem cell according to aspect 5, wherein said human pathology is selected from chronic myeloid leukaemia, B-cell acute lymphoblastic leukaemia, T-cell acute lymphoblastic leukaemia, acute myeloid leukaemia, chronic myeloid leukaemia, lymphoproliferative syndromes, multiple myeloma, liposarcoma, Ewing sarcoma, lung carcinoma, breast carcinoma, skin carcinoma, brain cancers, colon carcinomas, pancreatic carcinomas, prostate carcinomas, kidney carcinoma, etc,
7. Stem cell according to aspect 1, further comprising a marker.
8. Stem cell according to aspect 7, wherein said marker is a marker useful for the specific isolation and/or identification of cancer stem cells; or for the procurement of cancer stem cells; or for the differentiation of cancer stem cells from health stem cells.
9. Stem cell according to aspect 1, further comprising a reporter.
10. Stem cell according to aspect 9, wherein said reporter is a reporter useful for spatiotemporal identification of the onset, progression, dissemination and further physiopathological processes, for evaluating the effect of therapies by molecular imaging techniques, for diagnostic assays, drug discovery and development processes, for target identification and for improving the efficacy and reliability of all phases of the clinical development.
11. Use of a murine stem cell according to any one of aspects 1 to 10 as a biomarker for:
   detecting the presence of a gene created and/or activated by a genetic anomaly associated with a human pathology in a subject; or for
   assessing the risk or predisposition of a subject to develop a human pathology in a subject; or for
   determining the stage or severity of a human pathology in a subject; or for
   monitoring the effect of the therapy administered to a subject having a human pathology; or for
   designing an individualized therapy for a subject suffering from a human pathology; or for
   designing human clinical trials; or for
   diagnosis of cancer and/or specific processes and effects of cancer development, like cancer dissemination; or for
   patient selection for personalized therapeutics; or for
   therapeutic monitoring and evaluation of therapeutic benefits; or for
   drug discovery and pharmacokinetics guidance.
12. Use of a murine stem cell according to any one of aspects 1 to 10, for discovering, screening, searching, identifying, evaluating and validating targets for human pathologies; or for identifying specific genes related to self-renewal ability of cancer stem cells.
13. A method for detecting the presence of a gene created and/or activated by a genetic anomaly associated with a human pathology in a subject or for assessing the risk or predisposition of a subject to develop said pathology which comprises identifying a stem cell in a sample from said subject, said stem cell comprising a genetic anomaly associated with said human pathology of stem cell origin.
14. A method for determining the stage or severity of a human pathology in a subject or for monitoring the effect of the therapy administered to a subject having said pathology, which comprises identifying and quantifying the concentration of stem cells in a sample from said subject, said stem cells comprising a gene created and/or activated by a genetic anomaly associated with a human pathology, and comparing said concentration with that of a control sample or with a prior sample from said subject or with a prior sample from said subject taken before administering a therapy.
15. Method according to aspect 13 or 14, wherein said stem cell is a cancer stem cell.
16. Method according to any one of aspects 13 to 15, wherein said human pathology is a human pathology of stem cell origin.
17. A method for screening, searching, identifying, discovering, developing and/or evaluating compounds for treating a human pathology or for repositioning known drugs or combinations of compounds, which comprises contacting a candidate compound with a murine stem cell according to any one of aspects 1 to 10 and monitoring the response.
18. A method for designing an individualized therapy for a human suffering from a human pathology which comprises selecting a compound identified according to aspect 17, wherein said compound abolishes or slows down said cancer stem cells proliferation or differentiates or kills said stem cells, said compound being used as active principle in a pharmaceutical composition to be administered to said subject.
19. A method for designing human clinical trials which comprises:
   (i) selecting targets for murine stem cells according to any one of aspects 1 to 10 or molecular profiling said murine stem cells;
   (ii) validating and, optionally, optimizing, said target or said molecular profile in a disease state; and
   (iii) selecting responders vs non-responders.
20. A DNA construct comprising a gene created and/or activated by a genetic anomaly associated with a human pathology operatively bound to a promoter that directs the expression of said genetic anomaly in Sca-1⁺ cells, wherein said genetic anomaly is selected from (i) a nucleic acid comprising a BCL6 gene and (ii) a nucleic acid comprising a first nucleotide sequence coding for a kinase and a second nucleotide sequence comprising BCR-ABL^{p210}.
21. DNA construct according to aspect 20, wherein said gene created and/or activated by a genetic anomaly comprises a nucleic acid comprising a first nucleotide sequence coding for herpex simple thymidine kinase (HSV-tk), a second nucleotide sequence comprising BCR-ABL^{p210}, and a third nucleotide sequence comprising an internal ribosome-entry site (IRES) sequence, wherein 3′ end of said first nucleotide sequence is bound to the 5′ end of said third nucleotide sequence, and the 3′ end of said third nucleotide sequence is bound to the 5′ end of said second nucleotide sequence.
22. DNA construct according to any one of aspects 20 or 21, wherein said promoter that directs the expression of said gene created and/or activated by a genetic anomaly in Sca-1⁺ cells is the mouse promoter pLy-6E.1 or a functional fragment thereof.
23. A transgenic non-human mammal that contains in its genome a DNA construct according to the any one of aspects 20 to 22.
24. Transgenic non-human mammal according to aspect 23, wherein said mammal is a rodent, preferably, a mouse or a rat.
25. The progeny of a transgenic non-human mammal according to any one of aspects 23 or 24.
26. A cell line of a transgenic non-human mammal according to any one of aspects 23 or 24, said cell line containing in its genome a DNA construct according to any one of aspects 20 to 22.
27. Cell line according to aspect 26, wherein said cell line is a murine cell line.
28. A process for the preparation of a transgenic non-human mammal that possesses a genetic anomaly associated with a human pathology of stem cell origin, which comprises:
   a. introducing a DNA construct according to any one of aspects 20 to 23 into a fertilised oocyte of a non-human transgenic mammal;
   b. implanting said fertilised oocyte in a pseudopregnant wet nursing mother to produce descendents; and
   c. analysing said descendents to evaluate the existence of a genetic anomaly associated with a human pathology of stem cell origin.
29. Process according to aspect 28, wherein said non-human mammal is a rodent, preferably a mouse or a rat.

1. An animal solid tumour model, said model comprising a transgenic non-human mammal containing in its genome a DNA construct that comprises a gene created and/or activated by a genetic anomaly associated with human cancer operatively bound to a promoter that directs the expression of the gene in Sca1+ cells.

2. The animal according to claim 1, wherein said solid tumour is a mesenchymal cancer or epithelial cancer.

3. The animal model according to claim 2, wherein said solid tumour is a sarcoma, a carcinoma, multiple myeloma or a lymphoma.

4. The animal model according to claim 1, wherein said gene created and/or activated by a genetic anomaly is selected from the group consisting of BCR-ABLp210, BCR-ABLp190, Slug (SNAI2), Snail, HOX11, RHOM2/LMO-2, TAL1, Maf-B, FGFR, c-maf, MMSET, BCL6, BCL10, MALT1, cyclin D1, cyclin D3, SCL, LMO1, LMO2, TEL-AML1, E2A-HLF, E2A-Pbx1, TEL-ABL, AML1-ETO, FUS-DDIT3, EWS-WT1, EWS FLI1, EWSR1-DDIT3, FUS-ATF1, FUS-BBF2H7, K-RASv12 and Notch1.

5. The animal model according to claim 1, wherein the promoter that directs the expression of the gene in Sca-1+ cells is only active in the stem cell compartment and which is inactive once cells differentiate beyond the stem cell state.

6. The animal model according to claim 5, wherein the promoter is a pLy-6E.1 promoter, a Sca1 promoter, a musashi-1 promoter, a musashi-2 promoter, pLy6A gene promoter, Tmtsp gene promoter, c-kit gene promoter, CD34 gene promoter or Thy1 gene promoter.

7. The animal model according to claim 1, which uses a conditional system in which a recombinase is used conditionally to activate the gene.

8. The animal model according to claim 7, wherein the recombinase is the Cre recombinase.

9. The animal model according to claim 1, in which said activatable gene is KRASv12.

10. The animal model according to claim 1, which is a T-ALL model in which the animal develops lung adenocarcinoma or liver carcinoma.

11. The animal model according to claim 10, in which the activatable gene is Rhom2 or Hoxil.

12. The animal model according to claim 1, which is a B cell lymphoma model in which the animal develops lung adenocarcinoma or liver carcinoma.

13. The animal model according to claim 12, in which the activatable gene is BCL6.

14. The animal model according to claim 1, which is a CML model in which the animal develops lung adenocarcinoma (ADC), liver adenocarcinoma (ADC), fibrous histiocytoma, osteosarcoma or Sertoli cell tumours.

15. The animal model according to claim 14, in which the activatable gene is BCR-ABL^{p210}.

16. The animal model according to claim 1 which is a multiple myeloma model.

17. The animal model according to claim 16, in which the activatable gene is selected from the group consisting of Maf-B, FGF-R, c-maf and MMSET.

18. The animal model according to claim 16, in which the activatable gene is Maf-B.

19. A substantially pure culture of cancer stem cells (CSCs).

20. The culture according to claim 19, wherein said CSCs are Sca1+Lin−.

21. The A culture according to claim 20, wherein said CSCs have the potential to propagate and maintain cancer, particularly a solid tumour.

22. A substantially pure culture of cancer stem cells which have been isolated from an animal model according to claim 1.

23. A method of isolating a substantially pure culture of cancer stem cells, comprising isolating Sca1+ cells from a transgenic model according to claim 1 by selective enrichment.

24. A method for propagating a culture of stem cells of claim 19, said method comprising exposing the cancer stem cells in culture to a concentration of lysate produced from cells of at least one selected differentiated cell type, the concentration able to induce the cancer stem cells to propagate by preferentially undergoing either symmetric mitosis, whereby each dividing cancer stem cell produces two identical daughter cancer stem cells, or asymmetric mitosis.

25. A method of using an animal model according to claim 1 and/or a substantially pure culture of cancer stem cells selected from the group consisting of:
investigating and researching the cancer process;
identifying, classifying, isolating, purifying or describing CSC populations;
detecting the presence of a gene created and/or activated by a genetic anomaly associated with a human pathology in a subject;
assessing the risk or predisposition of a subject to develop a human pathology in a subject;
determining the stage or severity of a human pathology in a subject;
monitoring the effect of the therapy administered to a subject having a human pathology in a subject;
designing an individualized therapy for a subject suffering from a human pathology in a subject;
therapeutic monitoring and evaluation of therapeutic benefits;
designing human clinical trials and predicting the clinical outcome;
diagnosis of cancer and/or specific processes and effects of cancer development, like cancer dissemination;
patient selection for personalized therapeutics;
identifying drug repositioning with new and future drugs;
drug discovery and pharmacokinetics guidance; or for and
previous/early cancer detection and predicting the likelihood of clinical relapse.

26. A method for screening a subject for cancer, or a predisposition to cancer, comprising the step of testing a sample from the subject for the presence of a CSC as recited in claim 19.

27. A method for discovering, screening, searching, identifying, developing and/or evaluating compounds for treating a human solid tumour; or for repositioning a drug, which comprises contacting a candidate compound with an animal model according to claim 1 and/or a substantially pure culture of cancer stem cells, and monitoring the response.

28. The method according to claim 27, which comprises:
a) detecting the level of expression of an expression product of a gene expressed on CSCs in the presence of the candidate agent; and
b) comparing that level of expression with the level of expression in the absence of the candidate agent, wherein a reduction in expression indicates that the candidate agent modulates the level of expression of the expression product of the gene expressed in CSCs.

29. A method of treating cancer in a patient comprising ablating CSCs as defined in claim 19.

30. A process for the preparation of a transgenic non-human mammal that possesses a genetic anomaly associated with development of solid tumours, which comprises:
a) introducing into a fertilised oocyte of a non-human transgenic mammal a DNA construct that comprises a gene created and/or activated by a genetic anomaly associated with development of a solid tumour operatively bound to a promoter that directs the expression of said gene in Sca-1+ cells;
b) implanting said fertilised oocyte in a pseudopregnant wet nursing mother to produce descendents; and
c) analysing said descendants.

31. A method of generating an animal model comprising the step of introducing a DNA construct as described in claim 1 above within a defined inactive locus of the mouse genome.

32. The method according to claim 31 wherein said DNA construct is introduced by homologous recombination through ES cells.

33. The method according to claim 31, wherein the promoter of said DNA construct directs the expression of the gene in Sca-1+ cells is only active in the stem cell compartment and which is inactive once cells differentiate beyond the stem cell state and/or said DNA construct uses a conditional system in which a recombinase is used conditionally to activate the gene.

34. A method of stimulating an immune response to a CSC as defined in claim 19, comprising the steps of:
a) obtaining an enriched population of CSC;
b) treating the population to prevent cell division or replication;
c) administering the treated cells to a human or animal subject in an amount effective for inducing an immune response to cancer and/or CSC.

35. A method of analyzing a population of CSC as defined in claim 19 for gene and protein expression patterns as a source of CSC targets and biomarkers.
